SEQUENCE LISTING (30 sequences)

SEQ ID NO: 1 - DNA, 1700 bp, Homo sapiens
cttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtctgctgatatctgcagggacagagcattggggtgggggtaaggtgcatctgtttgaaaagtaaacgataaaatgtggattaaagtgcccagcacaaagcagatcctcaataaacatttcatttcccacccacactcgccagctcaccccatcatccctttcccttggtgccctccttttttttttatcctagtcattcttccctaatcttccacttgagtgtcaagctgaccttgctgatggtgacattgcacctggatgtactatccaatctgtgatgacattccctgctaataaaagacaacataactca

SEQ ID NO: 2 - protein, 206 aa, Homo sapiens
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 3 - protein, 159 aa, Homo sapiens
GAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 4 - protein, 144 aa, Homo sapiens
GVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 5 - protein, 126 aa, Homo sapiens
QAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 6 - protein, 103 aa, Homo sapiens
ESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 7 - protein, 75 aa, Homo sapiens
RTLKSFSTLANNFVLIVSQLQPSQENEMFSIRDSAHRRFLLFRRAFKQLDVEAALTKALGEVDILLTWMQKFYKL

SEQ ID NO: 8 - protein, 48 aa, Homo sapiens
MFSIRDSAHRRFLLFRRAFKQLDVEAALTKALGEVDILLTWMQKFYKL

SEQ ID NO: 9 - protein, 133 aa, Homo sapiens
GAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQL

SEQ ID NO: 10 - protein, 111 aa, Homo sapiens
GAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENE

SEQ ID NO: 11 - protein, 83 aa, Homo sapiens
GAQGQEFHFGPCQVKGVVPQKLWEAFWAVKDTMQAQDNITSARLLQQEVLQNVSDAESCYLVHTLLEFYLKTVFKNYHNRTVE

SEQ ID NO: 12 - protein, 57 aa, Homo sapiens
GAQGQEFHFGPCQVKGVVPQKLWEAFWAVKDTMQAQDNITSARLLQQEVLQNVSDAE

SEQ ID NO: 13 - protein, 39 aa, Homo sapiens
GVVPQKLWEAFWAVKDTMQAQDNITSARLLQQEVLQNVS

SEQ ID NO: 14 - protein, 50 aa, Homo sapiens
SCYLVHTLLEFYLKTVFKNYHNRTVEVRTLKSFSTLANNFVLIVSQLQPS

SEQ ID NO: 15 - protein, 43 aa, Homo sapiens
MFSIRDSAHRRFLLFRRAFKQLDVEAALTKALGEVDILLTWMQ

SEQ ID NO: 16 - DNA, 477 bp, Homo sapiens
ggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 17 - DNA, 432 bp, Homo sapiens
ggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 18 - DNA, 381 bp, Homo sapiens
atgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 19 - DNA, 309 bp, Homo sapiens
gagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 20 - DNA, 228 bp, Homo sapiens
gtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 21 - DNA, 144 bp, Homo sapiens
atgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 22 - DNA, 399 bp, Homo sapiens
ggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttg

SEQ ID NO: 23 - DNA, 333 bp, Homo sapiens
ggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgag

SEQ ID NO: 24 - DNA, 249 bp, Homo sapiens
ggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaa

SEQ ID NO: 25 - DNA, 171 bp, Homo sapiens
ggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgag

SEQ ID NO: 26 - DNA, 117 bp, Homo sapiens
ggggttgttccccagaaactgtgggaagccttctgggctgtgaaagacactatgcaagctcaggataacatcacgagtgcccggctgctgcagcaggaggttctgcagaacgtctcg

SEQ ID NO: 27 - DNA, 150 bp, Homo sapiens
agctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagt

SEQ ID NO: 28 - DNA, 129 bp, Homo sapiens
atgttttccatcagagacagtgcacacaggcggtttctgctattccggagagcattcaaacagttggacgtagaagcagctctgaccaaagcccttggggaagtggacattcttctgacctggatgcag

SEQ ID NO: 29 - protein, 53 aa, Homo sapiens
DAESCYLVHTLLEFYLKTVFKNYHNRTVEVRTLKSFSTLANNFVLIVSQLQPS

SEQ ID NO: 30 - protein, 126 aa, Homo sapiens
MQMVVLPCLGFTLLLWSQVSGAQGQEFHFGPCQVKGVVPQKLWEAFWAVKDTMQAQDNITSARLLQQEVLQNVSQENEMFSIRDSAHRRFLLFRRAFKQLDVEAALTKALGEVDILLTWMQKFYKL

(19) United States
(12) Patent Application Publication
(10) Pub. No.: US 2006/0292157 A1
(43) Pub. Date: Dec. 28, 2006

(54) MDA-7 protein variants having antiproliferative activity

(75) Inventors: Paul B. Fisher, Scarsdale, NY (US); Pankaj Gupta, New York, NY (US)

Correspondence Address: WILMER CUTLER PICKERING HALE AND DORR LLP;COLUMBIA UNIVERSITY, 399 PARK AVENUE, NEW YORK, NY, 10020, US

(73) Assignee: THE TRUSTEES OF COLUMBIA UNIVERSITY IN THE CITY OF NEW YORK, New York, NY (US)

(21) Appl. No.: 11/292,571

(22) Filed: Dec. 2, 2005

Related U.S. Application Data
(60) Provisional application No. 60/632,423, filed on Dec. 2, 2004.

(51) Int. Cl. A61K 39/395; A61K 38/17; A61K 48/00; C07K 16/30; C07K 14/82; C07H 21/04

(52) U.S. Cl. 424/155.1; 514/12; 514/44; 435/69.1; 435/320.1; 435/325; 530/350; 536/23.5; 530/388.8

(57) ABSTRACT

The invention relates to the mda-7 gene, its encoded protein and fragments of the protein. Several of these fragments of the MDA-7 protein exhibit antiproliferative activity and/or inhibited the activity of intact MDA-7. Accordingly, the invention provides, among other things, for methods and compositions that may be used in the treatment of disorders of cell proliferation, including cancer.

This application claims priority to U.S. application Ser. No. 60/632,423, which was filed on Dec. 2, 2004, and which is hereby incorporated by reference in its entirety.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

The subject matter of this provisional application was developed at least in part, using funds of National Institute of Health/National Cancer Institute Grant No. CA098712, so that the United States Government has certain rights herein.

### BACKGROUND

The melanoma differentiation-associated gene 7 (mda-7) gene was identified by a subtractive hybridization technique using cDNA libraries prepared from actively proliferating melanoma cells and from melanoma cells which had been induced to terminally differentiate by treatment with recombinant human fibroblast interferon (IFN-β) and the protein kinase C activator mezerein (U.S. Pat. No. 6,720,408 by Fisher et al., issued Apr. 13, 2004; Jiang and Fisher, 1993, Mol. Cell. Different. 1:285-299; Jiang et al., 1995, Oncogene 11:2477-2486). MDA-7 is a cytokine related to the Interleukin10 (IL-10) family. MDA-7 has been characterized as a protein having 206 amino acids with a size of 23.8 kDa and a sequence as set forth in SEQ ID NO:2 (Genbank Accession Number U16261; Jiang et al., 1995, Oncogene 11:2477-2486). MDA-7 has subsequently been renamed Interleukin-24 (IL-24), but will be referred to herein as MDA-7 or MDA-7/IL-24.

### SUMMARY OF THE INVENTION

The invention provides for polypeptides, which are fragments of MDA-7 protein and their use in modulating cell proliferation. The invention is based, at least in part, on the discovery that various subfragments of MDA-7 exhibit antiproliferative activity and/or inhibit the activity of intact MDA-7. Accordingly, the invention provides, among other things, for methods and compositions that may be used in the treatment of disorders of cell proliferation, including cancer.

The invention provides for MDA-7 variants including MDA-7 fragments that modulate cell proliferation. Some inhibit proliferation (as does MDA-7). Others have modest proliferation-enhancing effects.

One aspect of the invention is the surprising discovery that variants deriving from either the N-terminal or the C-terminal half of MDA-7 exhibit antiproliferative activity which approximates the level of antiproliferative activity of wild-type MDA-7.

Another aspect of the invention are methods of using the MDA-7 variants to modulate the activity of other interleukins such as IL-10, IL-20 or endogenously expressed mda-7 itself to thereby treat certain conditions.

The invention provides for compositions comprising the MDA-7 protein variants, and methods of using such variants for either (depending on the variant) inhibiting or promoting cell proliferation and/or differentiation.

The wild-type human MDA-7 protein sequence is 206 amino acids in length as follows: Met Asn Phe Gln Gln Arg Leu Gln Ser Leu Trp Thr Leu Ala Arg Pro Phe Cys Pro Pro Leu Leu Ala Thr Ala Ser Gln Met Gln Met Val Val Leu Pro Cys Leu Gly Phe Thr Leu Leu Leu Trp Ser Gln Val Ser Gly Ala Gln Gly Gln Glu Phe His Phe Gly Pro Cys Gln Val Lys Gly Val Val Pro Gln Lys Leu Trp Glu Ala Phe Trp Ala Val Lys Asp Thr Met Gln Ala Gln Asp Asn Ile Thr Ser Ala Arg Leu Leu Gln Gln Glu Val Leu Gln Asn Val Ser Asp Ala Glu Ser Cys Tyr Leu Val His Thr Leu Leu Glu Phe Tyr Leu Lys Thr Val Phe Lys Asn Tyr His Asn Arg Thr Val Glu Val Arg Thr Leu Lys Ser Phe Ser Thr Leu Ala Asn Asn Phe Val Leu Ile Val Ser Gln Leu Gln Pro Ser Gln Glu Asn Glu Met Phe Ser Ile Arg Asp Ser Ala His Arg Arg Phe Leu Leu Phe Arg Arg Ala Phe Lys Gln Leu Asp Val Glu Ala Ala Leu Thr Lys Ala Leu Gly Glu Val Asp Ile Leu Leu Thr Trp Met Gln Lys Phe Tyr Lys Leu (SEQ ID NO:2).

The invention provides for an isolated “MV1” polypeptide that is from about 145 amino acids to about 175 amino acids in length, and wherein the MV1 polypeptide is at least about 90 percent identical to a region from about amino acid 104 to about amino acid 206 of SEQ ID NO: 2. The invention also provides for an isolated “MV2” polypeptide that is from about 130 amino acids to about 155 amino acids in length, and wherein the MV2 polypeptide is at least about 90 percent identical to a region from about amino acid 63 to about amino acid 206 of SEQ ID NO: 2. The invention provides for an isolated “MV3” polypeptide that is from about 115 amino acids to about 138 amino acids in length, and wherein the MV3 polypeptide is at least about 90 percent identical to a region from about amino acid 80 to about amino acid 206 of SEQ ID NO: 2. The invention provides for an isolated “MV4” polypeptide that is from about 90 amino acids to about 110 amino acids in length, and wherein the MV4 polypeptide is at least about 90 percent identical to a region from about amino acid 104 to about amino acid 206 of SEQ ID NO: 2. The invention also provides for an isolated “MV5” polypeptide that is from about 70 amino acids to about 80 amino acids in length, and wherein the MV5 polypeptide is at least about 90 percent identical to a region from about amino acid 131 to about amino acid 206 of SEQ ID NO: 2. The invention further provides for an isolated “MV6” polypeptide that is from about 45 amino acids to about 55 amino acids in length, and wherein the MV6 polypeptide is at least about 90 percent identical to a region from about amino acid 159 to about amino acid 206 of SEQ ID NO: 2. The invention provides for an isolated “MV7” polypeptide that is from about 122 amino acids to about 146 amino acids in length, and wherein the MV7 polypeptide is at least about 90 percent identical to a region from about amino acid 48 to about amino acid 180 of SEQ ID NO: 2. The invention provides for an isolated “MV8” polypeptide that is from about 100 amino acids to about 120 amino acids in length, and wherein the MV8 polypeptide is at least about 90 percent identical to a region from about amino acid 48 to about amino acid 158 of SEQ ID NO: 2. The invention also provides for an isolated “MV9” polypeptide that is from about 75 amino acids to about 90 amino acids in length, and wherein the MV9 polypeptide is at least about 90 percent identical to a region from about amino acid 48 to about amino acid 130 of SEQ ID NO: 2. The invention also provides for an isolated “MV10” polypeptide that is from about 53 amino acids to about 63 amino acids in length, and wherein the MV10 polypeptide is at least about 90 percent identical to a region from about amino acid 48 to about amino acid 104 of SEQ ID NO: 2. The invention provides for an isolated “MVAB” polypeptide that is from about 32 amino acids to about 59 amino acids in length, and wherein the MVAB polypeptide is at least about 90 percent identical to a region from about amino acid 63 to about amino acid 101 of SEQ ID NO: 2. The invention also provides for an isolated “MVEF” polypeptide that is from about 35 amino acids to about 60 amino acids in length, and wherein the MVEF polypeptide is at least about 90 percent identical to a region from about amino acid 159 to about amino acid 201 of SEQ ID NO: 2.

In one embodiment, the invention provides for an MV1 polypeptide having an amino acid sequence of SEQ ID NO: 3. In one embodiment, the invention provides for an MV2 polypeptide having an amino acid sequence of SEQ ID NO: 4. In one embodiment, the invention provides for an MV3 polypeptide having an amino acid sequence of SEQ ID NO: 5. In one embodiment, the invention provides for an MV4 polypeptide having an amino acid sequence of SEQ ID NO: 6. In one embodiment, the invention provides for an MV5 polypeptide having an amino acid sequence of SEQ ID NO: 7. In one embodiment, the invention provides for an MV6 polypeptide having an amino acid sequence of SEQ ID NO: 8. In one embodiment, the invention provides for an MV7 polypeptide having an amino acid sequence of SEQ ID NO: 9. In one embodiment, the invention provides for an MV8 polypeptide having an amino acid sequence of SEQ ID NO: 10. In one embodiment, the invention provides for an MV9 polypeptide having an amino acid sequence of SEQ ID NO: 11. In one embodiment, the invention provides for an MV10 polypeptide having an amino acid sequence of SEQ ID NO: 12. In one embodiment, the invention provides for an MVAB polypeptide having an amino acid sequence of SEQ ID NO: 13. In one embodiment, the invention provides for an MVEF polypeptide having an amino acid sequence of SEQ ID NO: 14.

In one embodiment, these peptides of the invention can be linked to a stabilizing molecule. In another embodiment, the stabilizing molecule is a protein. In a further embodiment, the stabilizing molecule is a Glutathione-S-Transferase (GST) protein. The invention provides for a peptidomimetic of any of the polypeptides of the invention.

The invention also provides for an isolated nucleic acid encoding any of the polypeptides of the invention. The invention provides a nucleic acid encoding any of the polypeptides of the invention linked to a nucleic acid encoding a secretory peptide. The invention also provides for a nucleic acid under the control of a promoter and wherein the nucleic acid is linked to a conditionally replicable vector. In one embodiment, the nucleic acid encodes the MV4 polypeptide and where the secretory peptide comprises a secretory peptide of wild-type MDA-7, a cleavage signal peptide of gamma-interferon, an amino terminal leader sequence of mouse immunoglobulin light chain precursor. In one embodiment, the nucleic acid is linked to a conditionally replicating viral vector. In another embodiment, the nucleic acid is linked to a replication deficient viral vector. In another embodiment, the nucleic acid is contained within a liposome.

The invention also provides for a composition comprising the polypeptide of the invention. The invention also provides for a composition comprising a nucleic acid encoding the polypeptide of the invention.

The invention provides for a host cell containing a nucleic acid molecule encoding any of the polypeptides of the invention, wherein the nucleic acid is operably linked to a promoter and is expressed by the cell. In one embodiment, the host cell is a dendritic cell or a stem cell. The invention provides for a host cell containing a nucleic acid molecule encoding a polypeptide of the invention, linked to a second nucleic acid encoding a secretory peptide, wherein the first and second nucleic acids are operably linked to a promoter and the first and second nucleic acids are expressed and secreted by the cell. In one embodiment, the host cell is a dendritic cell or a stem cell.

The invention provides for a method of modulating proliferation of a cell, comprising administering, to the cell, an effective amount of a peptide of the invention. The invention also provides a method for modulating proliferation of a cell, comprising introducing into the cell, a nucleic acid of the invention. The invention also provides a method for inhibiting proliferation of a cell, the method comprising introducing into the cell an effective amount of the peptide of the invention. The invention also provides a method for inhibiting cell growth in a subject suffering from a cell proliferative disorder, the method comprising administering an effective, amount of the polypeptide of the invention. In one embodiment, the disorder is cancer. In another embodiment, the cell is a tumor cell. The invention provides a method for inhibiting proliferation of a cell, the method comprising introducing into the cell an effective amount of a nucleic acid encoding the peptide of the invention. In another embodiment, the invention provides for a method for inhibiting cell growth in a subject suffering from a cell proliferative disorder, the method comprising administering an effective, amount of a nucleic acid encoding the polypeptide of the invention. In one embodiment, the administration of the nucleic acid is via a nucleic acid vector, or a liposome. In another embodiment, the administration of the nucleic acid is via a virus, a replication defective viral vector, a replication conditional viral vector, a non-integrating virus, an adenovirus, AAV, VSV, Epstein Barr virus, measles, an integrating virus, a lentiviruses, a retroviruses, a plasmid, a synthetic delivery system, a liposome, a cationic polymer, a dendritic cell, a stem cell, or any combination thereof. In another embodiment, the method further comprises administering to the subject: a chemotherapeutic agent, a generator of free radicals, radiation therapy, an anti-ras agent, an anti-cancer antibody, or an anti-proliferative agent in combination with the polypeptide.

The invention provides for a method for treating inflammation in a subject, the method comprising administering, to the subject, an effective amount of the polypeptide of the invention. The invention also provides for a method for treating inflammation in a subject, the method comprising administering, to the subject, an effective amount of a nucleic acid encoding the polypeptide. In one embodiment, the method further comprises administering to the subject an anti-inflammatory agent in combination with the polypeptide.

The invention provides an antibody that specifically binds to the polypeptide of the invention. The invention also provides for an anti-idiotypic antibody that specifically binds to Bip/GRP78 in the same way that M4, a polypeptide having the amino acid sequence shown in SEQ ID NO: 6, binds to Bip/GRP78.

The invention provides for a polypeptide comprising M4, a polypeptide having the amino acid sequence shown in SEQ ID NO: 6, linked to an amino acid sequence of glutathione-S-transerfase.

The invention provides for a method for treating a tumor in a subject, the method comprising introducing into cells of a subject a nucleic acid encoding a polypeptide of the invention and a secretory peptide so that the cells express and secrete the polypeptide of the invention and wherein the expression and secretion of the polypeptide induces transformed-cell specific apoptosis. In one embodiment, the secretory peptide comprises a secretory peptide selected from the group consisting of: a secretory peptide of wild-type MDA-7, a cleavage signal peptide of gamma-interferon, and an amino terminal leader sequence of mouse immunoglobulin light chain precursor.

The invention provides for a method for inducing an anti-tumor bystander activity from a cell, the method comprising introducing into a cell a nucleic acid encoding a polypeptide of the invention, and a secretory peptide under the control of a promoter, so that the cell expresses and secretes the polypeptide of the invention, and wherein the expression and secretion of the polypeptide induces bystander anti-tumor activity. In one embodiment, the cell into which the nucleic acid is introduced is a normal cell.

The invention provides for a method for inducing anti-tumor apoptosis in a subject, the method comprising introducing into tumor cells of a subject a nucleic acid encoding a polypeptide of the invention, wherein the expression of the polypeptide induces anti-tumor apoptosis in the subject. The invention provides for a method for inhibiting angiogenesis in a tumor, the method comprising introducing into one or more cells of the tumor a nucleic acid encoding a polypeptide of the invention. The invention provides for a method for enhancing activity of an anti-cancer treatment regime of a subject, the method comprising administering to the subject a polypeptide of the invention in combination with the anti-cancer treatment regime. In one embodiment, the anti-cancer treatment regime comprises radiation, monoclonal antibody therapy, chemotherapy, or radioisotope therapy.

The invention provides for a method for identifying a compound capable of acting as a surrogate of M4 (SEQ ID NO: 6) by binding to Bip/GRP78 intracellularly, the method comprising: (a) contacting a cell with a test compound, wherein the cell expresses Bip/GRP78; (b) determining whether p38 MAPK is activated, wherein the activation of p38 MAPK indicates that the test compound acts as a surrogate of M4 (SEQ ID NO: 6). In one embodiment, the determination of whether p38 MAPK is activated comprises a determination of whether p38 MAPK is phosphorylated.

The invention provides for a method for inducing anti-tumor apoptosis in a subject, the method comprising introducing into tumor cells of a subject a nucleic acid encoding a polypeptide of SEQ ID NO:6, or the polypeptide of SEQ ID NO: 6 linked to glutathione-S-transferase, wherein the expression of the polypeptide induces anti-tumor apoptosis in the subject. The invention provides for a method for inhibiting angiogenesis in a tumor, the method comprising introducing into one or more cells of the tumor a nucleic acid encoding a polypeptide of SEQ ID NO:6, or the polypeptide of SEQ ID NO: 6 linked to glutathione-S-transferase. The invention provides for a method for enhancing activity of an anti-cancer treatment regime of a subject, the method comprising administering to the subject a polypeptide of SEQ I) NO:6, or the polypeptide of SEQ ID NO: 6 linked to glutathione-S-transferase in combination with the anti-cancer treatment regime. In one embodiment, the anti-cancer treatment regime comprises radiation, monoclonal antibody therapy, chemotherapy, or radioisotope therapy. The invention provides for a method for inducing an anti-tumor bystander activity from a cell, the method comprising introducing into a cell a nucleic acid encoding (a) a polypeptide of SEQ ID NO:6, or the polypeptide of SEQ ID NO: 6 linked to glutathione-S-transferase, and (b) a secretory polypeptide, both (a) and (b) under the control of a promoter, so that the cell expresses and secretes the polypeptide, and wherein the expression and secretion of the polypeptide induces bystander anti-tumor activity. The invention provides for a method for stimulating the immune system to produce additional cytokines, such as interferon gamma, TNF-alpha and interleukin-6 and downregulates TGF-beta, the method comprising administering to a subject in need thereof an effective amount of a polypeptide of SEQ ID NO:6 (M4), or the polypeptide of SEQ ID NO: 6 linked to glutathione-S-transferase. In one embodiment, the administration of the nucleic acid comprises administration via a virus, a replication defective viral vector, a replication conditional viral vector, a non-integrating virus, an adenovirus, AAV, VSV, Epstein Barr virus, measles, an integrating virus, a lentiviruses, a retroviruses, a plasmid, a synthetic delivery system, a liposome, a cationic polymer, a dendritic cell, a stem cell, or any combination thereof.

### BRIEF DESCRIPTION OF THE FIGURES

FIG. 1. This figure shows a schematic representation of MDA-7 variants M1-M10. each of the following is an embodiment of the invention. In this example, M1 is from amino acid 48 to 206, M2 is from amino acid 63 to 206, M3 is from amino acid 80 to 206, M4 is from amino acid 104 to 206, M5 is from amino acid 131 to 206, M6 is from amino acid 159 to 206, M7 is from amino acid 48 to 180; M8 is from amino acid 48 to 158, M9 is from amino acid 48 to 130, and M1O is from amino acid 48 to 104.

FIGS. 2A-B. These figures show bar diagrams that show the effect of the polypeptides of the invention on tumor growth (via a colony forming assay) and on cell killing (via a HeLa cell assay). FIG. 2A shows the effect of the polypeptides of the invention on a colony forming assay. FIG. 2B shows the polypeptides induced killing of HeLa cells.

FIG. 3. Effect of MDA-7 variants on colony formation of HeLa cells in monolayer colony formation.

FIG. 4. Effect of MDA-7 variants on monolayer colony formation of prostate carcinoma cell line DU145.

FIG. 5. This figure shows a schematic representation of domain clones of MDA-7 where AB domain includes amino acid 63-101 of wild-type MDA-7, where CD domain includes amino acids 105 to 154, and where the EF domain includes amino acids 159 to 201.

FIG. 6. Effect of different domains on killing activity in HeLa cells.

FIG. 7. Effect of MDA-7 domains on the killing effect of MDA-7 on DU145 cells.

FIGS. 8A-8E. Identifying the regions of functional activity of MDA-7/IL-24. FIG. 8A: Schematic representation of N-terminal deletion mutations generated in the MDA7/IL-24 gene. Fragments were cloned in the expression vector pREP4. FIGS. 8B, 8C and 8D: Effect of various deletion mutants on colony formation in cancer and normal cells. HeLa, DU-145 and P69 cells were transfected with different deletion constructs of MDA-7/IL-24 and the next day cells were subcultured and selected for colony formation ability in the presence of hygromycin for two weeks. Colonies >50 cells were counted and plotted. FIG. 8E: Expression of 3×Flag-tagged deletion constructs of MDA-7/IL-24 after transient transfection into HeLa cells.

FIGS. 9A-9G. M4 exhibits similar biological properties and activities as full-length MDA-7/IL-24. FIGS. 9A and 9B: Expression of MDA-7/IL-24 and M4 following adenovirus transduction. Adenoviruses expressing the full-length MDA-7/IL-24 or the M4 construct were analyzed for mRNA expression by Northern blotting (FIG. 9A) and protein expression by Western blotting (FIG. 9B). HeLa cells were infected with 100 pfu/cell of Ad.vec (lacking any gene insert), Ad.mda-7 (containing the full-length mda-7/IL-24 gene) or Ad.M4 (containing the M4 deletion construct) and mRNA and protein expression was determined 48 h postinfection. FIG. 9C: Ad.M4 reduces cell viability selectively in cancer cells. The indicated cell type was seeded in 96-well plates and infected with different pfu of Ad.vec, Ad.mda-7 or Ad.M4. After 5 days viability was assessed by MTT assay and plotted as the ratio to Ad.vec treatment. FIG. 9D: Cancer-specific colony formation inhibitory activity of Ad.M4. DU145, HeLa, T47D and P69 cells were infected with different pfu's of viruses and next day cells were subcultured at clonal densities in 60-mm dishes and allowed to form colonies for 2 weeks. After 2 weeks colonies >50 cells were counted and cloning efficiencies were calculated by dividing the number of colonies formed by the number of cells initially seeded. FIG. 9E: Ad.M4 induces apoptotic cell death in various cancer cells, but not in immortalized normal prostate cells. DU-145, HeLa, T47D and P69 cells were seeded in 6-well plates at a density of 2×105/well and the next day were infected with 100 pfu/cell of Ad.vec, Ad.mda-7 or Ad.M4. Twenty h later, the cells were trypsinized, washed twice with PBS and stained with allophycocyanin (APC) labeled Annexin V (BD Biosciences Pharrningen, San Diego, Calif.) and analyzed by flow cytometry. The amount of apoptotic cells was quantified using the FlowJo 6.3.1 program. FIGS. 9F and 9G: Full-length MDA-7/IL-24 and M4 localize in the ER. DU-145 (FIG. 9F) and P69 (FIG. 9G) cells were infected with 100 pfu/cell of Ad.M4 or Ad.mda-7. After 24 h post-infection, cells were fixed and MDA-7/IL-24 and M4 protein was detected by indirect immunofluorescence using anti-mda-7/IL-24 rabbit polyclonal antibodies. Colocalization was determined by using antibodies against the ER marker protein, Calregulin. Images of MDA-7/IL-24 and Calregulin were merged.

FIGS. 10A-10G. Mutation analysis of helix C and helix F of M4 and MDA-7/IL-24 confirms the importance of these regions in mediating cancer-selective growth inhibitory activity. FIG. 10A: Schematic representation of mutations generated in M4 targeting the C and F helices. Regions were either deleted or mutated and the resultant constructs were cloned in the vector pREP4. FIGS. 10B, 10C and 10D: Dependence on intact helices C and F in M4 for optimal cancer-selective growth inhibitory activity. HeLa (FIG. 10B), DU-145 (FIG. 10C) and P69 (FIG. 10D) were transfected with different deletion constructs of M4 and the next day cells were subcultured and selected for colony formation ability in the presence of hygromycin for two weeks. Colonies >50 cells were counted and plotted. FIG. 10E: Schematic of mutations at helices C and F of full-length MDA-7/IL-24. FIGS. 10F and 10G: Importance of the C and F helices of MDA-7/IL-24 in eliciting maximum growth inhibitory activity in cancer cells. HeLa and P69 cells were transfected with mutant constructs of MDA-7/IL-24. The next day cells were subcultured and selected for colony formation ability in the presence of hygromycin for two weeks. Colonies >50 cells were counted and plotted. Full-length MDA-7/IL-24 and M4 were used as controls.

FIGS. 11A-11E. Full-length MDA-7/IL-24 and M4 bind to BiP/GRP78. FIG. 11A: Coimmunoprecipitation of MDA-7/IL-24 and M4 with endogenous BiP/GRP78. HeLa cells were infected with 100 pfu/cell of Ad.mda-7, Ad.M4 or Ad.vec and immunoprecipitation analysis was performed 48 h later using BiP/GRP78 antibodies. FIG. 11B: Coimmunoprecipitation of Flag-tagged MDA-7/IL-24 or M4 with BiP/GRP78. Flag-tagged MDA-7/IL-24 or M4 and Myc-tagged BiP/GRP78 were cotransfected into HeLa cells. Forty-eight h post-transfection BiP/GRP78 was immunoprecipitated using BiP/GRP78 polyclonal antibodies. Samples were washed gently and separated on 12% SDS-PAGE and probed with Flag-M2 antibodies. FIG. 11 B confirms coimmunoprecipitation of MDA-7/IL-24 and M4 with BiP/GRP78, and shows the expression and immunoprecipitation profile of Myc-tagged BiP/GRP78 using myc antibodies. FIG. 11C: The MDA-7/IL-24 deletion mutants M1, M2 and M3 bind to BiP/GRP78. Samples, as shown in FIG. 11B, were immunoprecipitated using BiP/GRP78 polyclonal antibodies and co-immunoprecipitation was performed using Flag-M2 monoclonal antibodies. FIG. 11D: Confirmation of expression of Flag-Tagged C plus F helix mutants of MDA-7/IL-24 and M4. Expression of the indicated Flag-tagged mutants of MDA-7/IL24 and M4 at helices C and F, full-length MDA-7/IL-24 and M4 was confirmed with Flag-M2 monoclonal antibodies. FIG. 11E: MDA-7/IL-24 and M4 mutants at helices C and F do not bind BiP/GRP78. Co-immunoprecipitation experiments were performed using the mutants described in FIG. 11D and probed with Flag-M2 monoclonal antibodies.

FIGS. 12A-12D. Full-length MDA-7/IL-24 protein and proteins encoded by the M1, M2, M3 and M4 mutants co-localize with BiP/GRP78 in the ER. FIG. 12A: HeLa cells were transiently transfected with Flag-tagged full-length MDA-7/IL-24 or the indicated deletion mutants of MDA-7/IL-24. Twenty-four h post-transfection, cells were fixed and MDA-7/IL-24 protein was detected by indirect immunofluorescence using Flag M2 antibodies. Colocalization was determined by using antibodies against BiP/GRP78. Images of BiP/GRP78 and MDA-7/IL-24 were merged. FIG. 12B: MDA-7/IL-24, M1 and M4 induce phosphorylation of p38 MAPK, while inactive mutants M2 and M3 are devoid of activity. HeLa cells were transfected with constructs expressing full-length MDA-7/IL-24, specific deletion mutants of MDA-7/IL-24, M4 or specific sequence mutations in MDA-7/IL-24 or M4. Twenty-four hr post-transfection, cells were lysed and phosphorylation status of p38 was confirmed using phospho-p38 antibodies. Total p38 protein was also determined. FIG. 12C: Activation of Gadd34 and Gadd153 by full-length MDA-7/IL-24, M1 and M4. HeLa cells were transfected with the indicated constructs and 24 h later cells were lysed and RNA was isolated and Northern were blotting was performed using probes specific for Gadd34 and Gadd153. Full-length mda-7/IL-24 and the active mutants M1 and M4 induced Gadd34 and Gadd153 gene expression as confirmed by Northern blotting, while inactive mutants induced reduced Gadd34 as well as Gadd153 expression. The housekeeping gene gapdh was used to confirm equal loading of samples. FIG. 12D: Proposed model of the molecular mechanism of mda-7/IL-24-induced apoptosis. MDA-7/IL-24 protein (gray diamond) delivered by Ad.mda-7 localizes in the endoplasmic reticulum (ER) where it interacts with BIP/GRP78 that might result in activation of a yet unidentified molecule (X) and generation of “ER stress” that involves activation of p38 MAPK and induction of GADD family genes culminating in apoptosis. The secreted MDA-7/IL-24 interacts with its cognate receptors on the cell surface that activates a signaling cascade resulting in apoptosis. However, whether this signaling cascade also involves “ER stress” remains to be determined.

FIGS. 13A-13B. Ad.M4 displays potent antitumor activity in vivo in an experimental human breast tumor xenograft nude mouse model. T47D human breast carcinoma cells were injected subcutaneously in the left and right flanks of male athymic nude mice. After tumors were formed, intratumoral injections of different Ad were given only to the tumors on the left side at a dose of 1×10⁸ pfu. Injections were given three times a week in the first week followed by two injections for the next two weeks. At the end of the experiment the animals were sacrificed and the tumors were removed and weighed. FIG. 13A describes the tumor volume on the left side while FIG. 13B describes the tumor volume on the right side.

FIG. 14. Equivalent bystander activity of mda-7/WL-24 and M4 as measured in HeLa cells, DU-145 cells and A549 cells.

### DETAILED DESCRIPTION OF THE INVENTION

When the mda-7 gene was introduced into a wide spectrum of human cancers, growth of cancer cells was inhibited (U.S. Pat. No. 5,710,137 by Fisher, issued Jan. 20, 1998; U.S. Pat. No. 6,355,622 by Fisher, issued Mar. 12, 2002; Jiang et al., 1996, Proc. Natl. Acad. Sci. U.S.A. 93:9160-9165; Su et al., 1998, Proc. Natl. Acad. Sci. U.S.A. 95:14400-14405; Madireddi et al., 2000, Adv. Exptl. Med. Biol. 465:239-261; Saeki et al., 2000, Gene Ther. 7:2051-2057; Huang et al., 2001, Oncogene 20:7051-63; Mhashilkar et al., 2001, Mol. Med. 7:271-282; Cao et al., 2002, Mol. Med. 8:869-876; Kawabe et al., 2002, Mol. Ther. 6:637-644; Lebedeva et al., 2002, Oncogene 21:708-718; Pataer et al., 2002, Cancer Res. 62:2239-2243; Saeki et al., 2002, Oncogene 21:4558-4566; Sarkaret al., 2002, Proc. Natl. Acad. Sci. U.S.A. 99:10054-10059; Su et al., 2001, Proc. Natl. Acad. Sci. U.S.A. 98:10332-10337; Pataer et al., 2003, J. Thorac. Cardiovasc. Surg. 125:1328-1335; Sauane et al., 2003, Cytokine Growth Factor Rev. 14:35-51; Sauane et al., 2003, J. Cell. Physiol. 196:334-345; Su et al., 2003 Oncogene 22:1164-1180; Yacoub et al., 2003, Mol. Cancer Therapeut. 2:623-632). mda-7 has been observed to suppress growth in cancer cells which either do not express, or which contain defects in, both retinoblastoma (“Rb”) and p53 tumor suppressor genes, indicating that mda-7 mediated growth inhibition does not depend on these elements (Jiang et al., 1996, Proc. Natl. Acad. Sci. U.S.A. 93:9160-9165). In contrast to the anti-proliferative effect on various cancer cells, no significant growth inhibitory effect was apparent when this gene was introduced into normal human fibroblast or epithelial cells (Jiang et al., 1996, Proc. Natl. Acad. Sci. U.S.A. 93:9160-9165; Madireddi et al., 2000, Adv. Exptl. Med. Biol. 465:239-261; Saeki et al., 2000, Gene Ther. 7:2051-2057; Mhashilkar et al., 2001, Mol. Med. 7:271-282).

The cancer-selective activity of mda-7 in most cases appears not to be a consequence of differences in mda-7 expression, protein production or secretion following infection with Ad.mda-7 (Mhashilkar et al., 2001, Mol. Med. 7:271-282; Lebedeva et al., 2002, Oncogene 21:708-718; Su et al., 2003 Oncogene 22:1164-1180). In specific cell types, including breast, pancreatic and prostate carcinomas, melanomas and malignant gliomas, induction of apoptosis correlates with changes in the ratio of pro-apoptotic proteins (such as Bax and Bak) to anti-apoptotic proteins (such as Bcl-2 and Bcl-xL), thereby shifting the balance from survival to programmed cell death (International Patent Application No. PCT/US03/21237, by Fisher et al., published as WO 04/005481 on Jan. 15, 2004 by the Trustees of Columbia University; Saeki et al., 2000, Gene Ther. 7:2051-2057; Lebedeva et al., 2002, Oncogene 21:708-718; Su et al., 2003 Oncogene 22:1164-1180). Changes in cell cycle are also evident in some, but not all, cancer cells infected with Ad.mda-7 (Saeki et al., 2000, Gene Ther. 7:2051-2057; Lebedeva et al., 2002, Oncogene 21:708-718; Su et al., 2003 Oncogene 22:1164-1180). A cell cycle change seen in Ad.mda-7-infected melanomas, non-small cell lung carcinomas, prostate carcinomas and certain malignant gliomas is an increase in the proportion of cells in the G2/M phase (Saeki et al., 2000, Gene Ther. 7:2051-2057; Lebedevaet al., 2002, Oncogene 21:708-718; Su et al., 2003 Oncogene 22:1164-1180). Apoptosis induction associates with activation of the caspase cascade in specific tumor systems, including activation of caspase-9 and caspase-3 and cleavage of PARP, a caspase substrate (Saeki et al., 2000, Gene Ther. 7:2051-2057; Mhashilkar et al., 2001, Mol. Med. 7:271-282; Pataer et al., 2002, Cancer Res. 62:2239-2243).

As an approach to more efficiently administer mda-7 and to begin to define the mechanism by which mda-7 selectively affects cancer cell proliferation, a replication-incompetent adenovirus (Ad.mda-7) was constructed (International Patent Application No. PCT/US02/26454, by Fisher et al., published as WO 03/016499 on Feb. 27, 2003 by the Trustees of Columbia University; Su et al., 1998, Proc. Natl. Acad. Sci. U.S.A. 95:14400-14405). Studies in the context of breast carcinoma cells demonstrated that Ad.mda-7 selectively induced growth suppression and this process occurred by induction of programmed cell death (apoptosis) (Su et al., 1998, Proc. Natl. Acad. Sci. U.S.A. 95:14400-14405). In contrast, as observed with transfection, infection of normal mammary epithelial and HBL-100 cells with Ad.mda-7 did not significantly affect growth or reduce viability. Analysis of the potential mechanism by which mda-7 induced apoptosis indicated up-regulation of the pro-apoptotic molecule Bax uniquely in breast cancer cells, irrespective of their p53 gene status. Additionally, the level of the pro-apoptotic protein Bcl-2 was reduced in multiple breast carcinoma cells following Ad.mda-7 infection.

It has further been observed that the anti-cancer effects of mda-7 gene or MDA-7 protein could be enhanced by concurrent exposure to radiation and/or other free radical generators (International Patent Application No. PCT/US03/28512, by Fisher et al., published as WO 04/060269 on Jul. 22, 2004 by the Trustees of Columbia University and Virginia Commonwealth University). For example, Yacoub et al. (2003, Mol. Cancer Ther. 2:623-632) report that MDA-7 protein, in combination with agents that generate free radicals, can selectively inhibit the proliferation of renal carcinoma cells relative to their non-malignant counterparts.

As mentioned above, native (wild-type) MDA-7 protein has 206 amino acids (SEQ ID NO:2). It would be desirable to identify a smaller fragment of MDA-7 that could be used therapeutically, as techniques for administering peptide therapies are more refined than techniques for administering protein. The invention addresses this need by providing biologically active MDA-7 variants that are smaller than the native protein.

The following references are heerby incorporated by reference: Fisher, Cancer Res 65(22):10128-10138 (2005); Lebedeva et al., Mol Therapy 11(1):4-18 (2005); and Su et al., Oncogene 24:7552-7566 (2005).

For clarity of description, and not by way of limitation, the detailed description of the invention is divided into the following subsections:

(i) MDA-7 variants;

(ii) assays to confirm MDA-7 variant activity;

(iii) use of a MDA-7 variant as a gene therapy;

(iv) use of a MDA-7 variant as a peptide therapy;

(v) combined therapy using MDA-7 variants; and

(vi) conditions which may be treated.

As used herein, mda-7 (italicized and lower case letters) refers to the gene or a corresponding nucleic acid; MDA-7 (all capital letters) refers to a protein, Mda-7 (initial capital letter only) refers collectively to nucleic acids, proteins and peptides.

MDA-7 Protein Variants

The terms “peptide,” “polypeptide,” and “protein” are used interchangeably to refer to a polymer of amino acid residues, and are not limited to a minimum length. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

By “isolated” is meant, when referring to a polynucleotide or polypeptide of the invention, that the indicated molecule is substantially separated, e.g., from the whole organism in which the molecule is found or from the cell culture in which the antibody is produced, or is present in the substantial absence of other biological macromolecules of the same type. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

A “modulator” of the polypeptides or polynucleotides or an “agent” herein is an agonist or antagonist that interferes with the binding or activity of such polypeptides or polynucleotides. Such modulators or agents include, for example, polypeptide variants, whether agonist or antagonist; antibodies, whether agonist or antagonist; soluble receptors, usually antagonists; small molecule drugs, whether agonist or antagonist; RNAi, usually an antagonist; antisense molecules, usually an antagonist; and ribozymes, usually an antagonist. In some embodiments, an agent is a subject polypeptide, where the subject polypeptide itself is administered to an individual. In some embodiments, an agent is an antibody specific for a subject “target” polypeptide. In some embodiments, an agent is a chemical compound such as a small molecule that may be useful as an orally available drug. Such modulation includes the recruitment of other molecules that directly effect the modulation. For example, an antibody that modulates the activity of a subject polypeptide that is a receptor on a cell surface may bind to the receptor and fix complement, activating the complement cascade and resulting in lysis of the cell. An agent which modulates a biological activity of a subject polypeptide or polynucleotide increases or decreases the activity or binding at least about 10%, at least about 15%, at least about 20%, at least about 25%, at least about 50%, at least about 80%, or at least about 2-fold, at least about 5-fold, or at least about 10-fold or more when compared to a suitable control.

The use of the word “a” or “an” when used in conjunction with the term “comprising” in the claims and/or the specification may mean “one,” but it is also consistent with the meaning of “one or more,” “at least one,” and “one or more than one.”

The term “MDA-7” as used herein refers to a protein having essentially the amino acid sequence set forth as SEQ ID NO:2, having Genbank Accession Number U16261. A nucleic acid encoding MDA-7 may have the coding sequence as set forth in SEQ ID NO:1, Genbank Accession No. U16261, or another sequence which, when translated, produces a protein having essentially the same amino acid sequence as SEQ ID NO:2. It should be noted that the portion of the nucleic acid sequence presented as SEQ ID NO:1 which constitutes the protein encoding region extends from nucleotide 275 to nucleotide 895. The definition of Mda-7 embraces functional equivalents of the nucleic acid and protein which vary in insignificant ways from the native molecules; for example, it includes isolated nucleic acids which hybridize to the nucleic acid sequence set forth as SEQ ID NO:1 under stringent hybridization conditions, e.g., hybridization in 0.5 M NaHPO₄, 7 percent sodium dodecyl sulfate (“SDS”), 1 mM ethylenediamine tetraacetic acid (“EDTA”) at 65° C., and washing in 0.1×SSC/0.1 percent SDS at 68° C. (Ausubel et al., 1989, Current Protocols in Molecular Biology, Vol. I, Green Publishing Associates, Inc., and John Wiley & Sons, Inc. New York, at p. 2.10.3), as well as the proteins encoded by such hybridizing sequences. The nucleic acid molecule can be a cDNA molecule, a genomic DNA molecule, a cRNA molecule, a siRNA molecule, an RNAi molecule, an mRNA molecule, an anti-sense molecule, and/or a ribozyme. It can also be the complement of any of these. The definition of Mda-7 also includes nucleic acids and proteins which are at least 80, 90, or 95 percent homologous to SEQ ID NOS: 1 and 2 respectively, where homology is determined using standard software, (see below). It also includes nucleic acids having essentially the sequence set forth as SEQ ID NO:1, but modified to contain restriction sites appropriate for insertion into a particular expression vector, and proteins or peptides modified to contain residues that alter stability or cellular compartmentalization.

The term “MDA-7 variant,” as used herein, refers to a polypeptide, wherein the sequence of said polypeptide has at least about 80 percent, or at least about 85, 90 or 95 percent sequence identity to the corresponding sequence of wild-type MDA-7 (SEQ ID NO:2). Percent identity is calculated by determining the ratio of identical amino acids divided by total amino acids, and then multiplying the ratio by 100. In one embodiment, the MDA-7 protein variant can be a polypeptide having up to about 180 amino acids, or up to about 110 amino acids, or from about 40 to about 70 amino acids. The amino acid sequence of native (i.e., wild type) MDA-7 is set forth in SEQ ID NO:2.

Percent identity between sequences can be manually determined or can be determined using software and a computer, which can determine homology and identity. For example, such software is known in the art, e.g., the GCG package, NCBI BLAST or MacVector.

In one embodiment, an MDA-7 protein variant comprises a polypeptide having an amino acid sequence that corresponds to SEQ ID NO: 2 from about amino acid 50 to about amino acid 149, wherein the amino acid sequence that corresponds to SEQ ID NO:2 of the variant has at least 90 percent identical residues to SEQ ID NO:2. The invention provides for MDA-7 protein variants that correspond to the wild-type sequence of MDA-7 (SEQ ID NO:2) such that the MDA-7 protein variant has about 90 percent identical residues with the wild-type sequence. In another embodiment, the MDA-7 protein variant comprises a polypeptide comprising (a) a first amino acid sequence that corresponds to SEQ ID NO: 2 from about amino acid 50 to about amino acid 149, wherein the first amino acid sequence has at least 90 percent identical residues to SEQ ID NO:2, (b) and a second amino acid sequence from about 10 to about 50 amino acid residues. In one embodiment the second amino acid sequence has no identity to SEQ ID NO:2. In another embodiment, the second amino acid sequence has up to 50 percent identity to SEQ ID NO:2.

The invention also provides for an MDA-7 polypeptide comprising an amino acid sequence that has about 90% identity to: from about amino acid 50 to about amino acid 149 of SEQ ID NO: 2. In one embodiment, the MDA-7 polypeptide is not the following polypeptide: Asp Ala Glu Ser Cys Tyr Leu Val His Thr Leu Leu Glu Phe Tyr Leu Lys Thr Val Phe Lys Asn Tyr His Asn Arg Thr Val Glu Val Arg Thr Leu Lys Ser Phe Ser Thr Leu Ala Asn Asn Phe Val Leu Ile Val Ser Gln Leu Gln Pro Ser. In another embodiment, the MDA-7 polypeptide is not the following polypeptide: Met Gln Met Val Val Leu Pro Cys Leu Gly Phe Thr Leu Leu Leu Trp Ser Gln Val Ser Gly Ala Gln Gly Gln Glu Phe His Phe Gly Pro Cys Gln Val Lys Gly Val Val Pro Gln Lys Leu Trp Glu Ala Phe Trp Ala Val Lys Asp Thr Met Gln Ala Gln Asp Asn Be Thr Ser Ala Arg Leu Leu Gln Gln Glu Val Leu Gln Asn Val Ser Gln Glu Asn Glu Met Phe Ser Ile Arg Asp Ser Ala His Arg Arg Phe Leu Leu Phe Arg Arg Ala Phe Lys Gln Leu Asp Val Glu Ala Ala Leu Thr Lys Ala Leu Gly Glu Val Asp Ile Leu Leu Thr Trp Met Gln Lys Phe Tyr Lys Leu.

In non-limiting embodiments of the invention, the invention provides for the following MDA-7 variants (“MVX” polypeptides):

MV1, a protein having anti-proliferative activity, corresponds to amino acid residues 48-206 of wild-type MDA-7. In one embodiment, MV1 is a protein having an amino acid sequence from about 130 amino acids to about 190 amino acids in length. In another embodiment, MV1 is a protein having an amino acid sequence from about 145 amino acids to about 175 amino acids in length. In another embodiment, MV1 is a protein having about 160 amino acids. In one embodiment, the MV1 protein has at least about 80 percent identity to amino acids from about 48 to about 206 of SEQ ID NO:2. In another embodiment, the MV1 protein has at least about 85 percent identity to amino acids from about 48 to about 206 of SEQ ID NO:2. In another embodiment, the MV1 protein has at least about 90 percent identity to amino acids from about 48 to about 206 of SEQ ID NO:2. In another embodiment, the MV1 protein has at least about 95 percent identity to amino acids from about 48 to about 206 of SEQ ID NO:2. In another embodiment, the MV1 protein has at least about 99 percent identity to amino acids from about 48 to about 206 of SEQ ID NO:2.

MV2, a protein having slight proliferative activity, corresponds to amino acid residues 63-206 of wild-type MDA-7. In one embodiment, MV2 is a protein having an amino acid sequence from about 115 amino acids to about 170 amino acids in length. In another embodiment, MV2 is a protein having an amino acid sequence from about 130 amino acids to about 155 amino acids in length. In another embodiment, MV2 is a protein having about 144 amino acids. In one embodiment, the MV2 protein has at least about 80 percent identity to amino acids from about 63 to about 206 of SEQ ID NO:2. In another embodiment, the MV2 protein has at least about 85 percent identity to amino acids from about 63 to about 206 of SEQ ID NO:2. In another embodiment, the MV2 protein has at least about 90 percent identity to amino acids from about 63 to about 206 of SEQ ID NO:2. In another embodiment, the MV2 protein has at least about 95 percent identity to amino acids from about 63 to about 206 of SEQ ID NO:2. In another embodiment, the MV2 protein has at least about 99 percent identity to amino acids from about 63 to about 206 of SEQ ID NO:2.

MV3, a protein having slight proliferative activity, corresponds to amino acid residues 80-206 of wild-type MDA-7. In one embodiment, MV3 is a protein having an amino acid sequence from about 105 amino acids to about 150 amino acids in length. In another embodiment, MV3 is a protein having an amino acid sequence from about 115 amino acids to about 138 amino acids in length. In another embodiment, MV3 is a protein having about 127 amino acids. In one embodiment, the MV3 protein has at least about 80 percent identity to amino acids from about 80 to about 206 of SEQ ID NO:2. In another embodiment, the MV3 protein has at least about 85 percent identity to amino acids from about 80 to about 206 of SEQ ID NO:2. In another embodiment, the MV3 protein has at least about 90 percent identity to amino acids from about 80 to about 206 of SEQ ID NO:2. In another embodiment, the MV1 protein has at least about 95 percent identity to amino acids from about 48 to about 206 of SEQ ID NO:2. In another embodiment, the MV3 protein has at least about 99 percent identity to amino acids from about 80 to about 206 of SEQ ID NO:2.

MV4, a protein having anti-proliferative activity, corresponds to amino acid residues 104-206 of wild-type MDA-7. In one embodiment, MV4 is a protein having an amino acid sequence from about 80 amino acids to about 120 amino acids in length. In another embodiment, MV4 is a protein having an amino acid sequence from about 90 amino acids to about 110 amino acids in length. In another embodiment, MV4 is a protein having about 103 amino acids. In one embodiment, the MV4 protein has at least about 80 percent identity to amino acids from about 104 to about 206 of SEQ ID NO:2. In another embodiment, the MV4 protein has at least about 85 percent identity to amino acids from about 104 to about 206 of SEQ ID NO:2. In another embodiment, the MV4 protein has at least about 90 percent identity to amino acids from about 104 to about 206 of SEQ ID NO:2. In another embodiment, the MV4 protein has at least about 95 percent identity to amino acids from about 104 to about 206 of SEQ ID NO:2. In another embodiment, the MV4 protein has at least about 99 percent identity to amino acids from about 104 to about 206 of SEQ ID NO:2.

MV5, a peptide having slight proliferative activity, corresponds to amino acid residues 131-206 of wild-type MDA-7. In one embodiment, MV5 is a protein having an amino acid sequence from about 60 amino acids to about 90 amino acids in length. In another embodiment, MV5 is a protein having an amino acid sequence from about 70 amino acids to about 80 amino acids in length. In another embodiment, MV5 is a protein having about 77 amino acids. In one embodiment, the MV5 protein has at least about 80 percent identity to amino acids from about 131 to about 206 of SEQ ID NO:2. In another embodiment, the MV5 protein has at least about 85 percent identity to amino acids from about 131 to about 206 of SEQ ID NO:2. In another embodiment, the MV5 protein has at least about 90 percent identity to amino acids from about 131 to about 206 of SEQ ID NO:2. In another embodiment, the MV5 protein has at least about 95 percent identity to amino acids from about 131 to about 206 of SEQ ID NO:2. In another embodiment, the MV5 protein has at least about 99 percent identity to amino acids from about 131 to about 206 of SEQ ID NO:2.

MV6, a peptide having slight proliferative activity, corresponds to amino acid residues 159-206 of wild-type MDA-7. In one embodiment, MV6 is a protein having an amino acid sequence from about 40 amino acids to about 60 amino acids in length. In another embodiment, MV6 is a protein having an amino acid sequence from about 45 amino acids to about 55 amino acids in length. In another embodiment, MV6 is a protein having about 48 amino acids. In one embodiment, the MV6 protein has at least about 80 percent identity to amino acids from about 159 to about 206 of SEQ ID NO:2. In another embodiment, the MV6 protein has at least about 85 percent identity to amino acids from about 159 to about 206 of SEQ ID NO:2. In another embodiment, the MV6 protein has at least about 90 percent identity to amino acids from about 159 to about 206 of SEQ ID NO:2. In another embodiment, the MV6 protein has at least about 95 percent identity to amino acids from about 159 to about 206 of SEQ ID NO:2. In another embodiment, the MV6 protein has at least about 99 percent identity to amino acids from about 159 to about 206 of SEQ ID NO:2.

MV7, a protein having slight proliferative activity, corresponds to amino acid residues 48-180 of wild-type MDA-7. In one embodiment, MV7 is a protein having an amino acid sequence from about 110 amino acids to about 160 amino acids in length. In another embodiment, MV7 is a protein having an amino acid sequence from about 122 amino acids to about 146 amino acids in length. In another embodiment, MV7 is a protein having about 134 amino acids. In one embodiment, the MV7 protein has at least about 80 percent identity to amino acids from about 48 to about 180 of SEQ ID NO:2. In another embodiment, the MV7 protein has at least about 85 percent identity to amino acids from about 48 to about 180 of SEQ ID NO:2. In another embodiment, the MV7 protein has at least about 90 percent identity to amino acids from about 48 to about 180 of SEQ ID NO:2. In another embodiment, the MV7 protein has at least about 95 percent identity to amino acids from about 48 to about 180 of SEQ ID NO:2. In another embodiment, the MV7 protein has at least about 99 percent identity to amino acids from about 48 to about 180 of SEQ ID NO:2.

MV8, a protein having slight proliferative activity, corresponds to amino acid residues 48-158 of wild-type MDA-7. In one embodiment, MV8 is a protein having an amino acid sequence from about 90 amino acids to about 130 amino acids in length. In another embodiment, MV8 is a protein having an amino acid sequence from about 100 amino acids to about 120 amino acids in length. In another embodiment, MV8 is a protein having about 112 amino acids. In one embodiment, the MV8 protein has at least about 80 percent identity to amino acids from about 48 to about 158 of SEQ ID NO:2. In another embodiment, the MV8 protein has at least about 85 percent identity to amino acids from about 48 to about 158 of SEQ ID NO:2. In another embodiment, the MV8 protein has at least about 90 percent identity to amino acids from about 48 to about 158 of SEQ ID NO:2. In another embodiment, the MV8 protein has at least about 95 percent identity to amino acids from about 48 to about 158 of SEQ ID NO:2. In another embodiment, the MV8 protein has at least about 99 percent identity to amino acids from about 48 to about 158 of SEQ ID NO:2.

MV9, a peptide having slight proliferative activity, corresponds to amino acid residues 48-130 of wild-type MDA-7. In one embodiment, MV9 is a protein having an amino acid sequence from about 70 amino acids to about 100 amino acids in length. In another embodiment, MV9 is a protein having an amino acid sequence from about 75 amino acids to about 90 amino acids in length. In another embodiment, MV9 is a protein having about 84 amino acids. In one embodiment, the MV9 protein has at least about 80 percent identity to amino acids from about 48 to about 130 of SEQ ID NO:2. In another embodiment, the MV9 protein has at least about 85 percent identity to amino acids from about 48 to about 130 of SEQ ID NO:2. In another embodiment, the MV9 protein has at least about 90 percent identity to amino acids from about 48 to about 130 of SEQ ID NO:2. In another embodiment, the MV9 protein has at least about 95 percent identity to amino acids from about 48 to about 130 of SEQ ID NO:2. In another embodiment, the MV9 protein has at least about 99 percent identity to amino acids from about 48 to about 130 of SEQ ID NO:2.

MV10, a peptide having anti-proliferative activity, corresponds to amino acid residues 48-104 of wild-type MDA-7. In one embodiment, MV10 is a protein having an amino acid sequence from about 50 amino acids to about 70 amino acids in length. In another embodiment, MV10 is a protein having an amino acid sequence from about 53 amino acids to about 63 amino acids in length. In another embodiment, MV10 is a protein having about 58 amino acids. In one embodiment, the MV10 protein has at least about 80 percent identity to amino acids from about 48 to about 104 of SEQ ID NO:2. In another embodiment, the MV10 protein has at least about 85 percent identity to amino acids from about 48 to about 104 of SEQ ID NO:2. In another embodiment, the MV10 protein has at least about 90 percent identity to amino acids from about 48 to about 104 of SEQ ID NO:2. In another embodiment, the MV10 protein has at least about 95 percent identity to amino acids from about 48 to about 104 of SEQ ID NO:2. In another embodiment, the MV10 protein has at least about 99 percent identity to amino acids from about 48 to about 104 of SEQ ID NO:2.

MVAB (AB domain), a peptide having anti-proliferative activity, corresponds to amino acid residues 63-101 of wild-type MDA-7. In one embodiment, MVAB is a protein having an amino acid sequence from about 32 amino acids to about 59 amino acids in length. In another embodiment, MVAB is a protein having an amino acid sequence from about 35 amino acids to about 46 amino acids in length. In another embodiment, MVAB is a protein having about 39 amino acids. In one embodiment, the MVAB protein has at least about 80 percent identity to amino acids from about 63 to about 101 of SEQ ID NO:2. In another embodiment, the MVAB protein has at least about 85 percent identity to amino acids from about 63 to about 101 of SEQ ID NO:2. In another embodiment, the MVAB protein has at least about 90 percent identity to amino acids from about 63 to about 101 of SEQ ID NO:2. In another embodiment, the MVAB protein has at least about 95 percent identity to amino acids from about 63 to about 101 of SEQ ID NO:2. In another embodiment, the MVAB protein has at least about 99 percent identity to amino acids from about 63 to about 101 of SEQ ID NO:2.

MVCD (CD domain), a peptide having anti-proliferative activity, corresponds to amino acid residues 105-154 of wild-type MDA-7. In one embodiment, MVCD is a protein having an amino acid sequence from about 35 amino acids to about 100 amino acids in length. In another embodiment, MVCD is a protein having an amino acid sequence from about 42 amino acids to about 85 amino acids in length. In another embodiment, MVCD is a protein having about 50 amino acids. In one embodiment, the MVCD protein has at least about 80 percent identity to amino acids from about 105 to about 154 of SEQ ID NO:2. In another embodiment, the MVCD protein has at least about 85 percent identity to amino acids from about 105 to about 154 of SEQ ID NO:2. In another embodiment, the MVCD protein has at least about 90 percent identity to amino acids from about 105 to about 154 of SEQ ID NO:2. In another embodiment, the MVCD protein has at least about 95 percent identity to amino acids from about 105 to about 154 of SEQ ID NO:2. In another embodiment, the MVCD protein has at least about 99 percent identity to amino acids from about 105 to about 154 of SEQ ID NO:2.

MVEF (EF domain), a peptide having anti-proliferative activity, corresponds to amino acid residues 159-201 of wild-type MDA-7. In one embodiment, MVEF is a protein having an amino acid sequence from about 35 amino acids to about 60 amino acids in length. In another embodiment, MVEF is a protein having an amino acid sequence from about 40 amino acids to about 56 amino acids in length. In another embodiment, MVEF is a protein having about 43 amino acids. In one embodiment, the MVEF protein has at least about 80 percent identity to amino acids from about 159 to about 201 of SEQ ID NO:2. In another embodiment, the MVEF protein has at least about 85 percent identity to amino acids from about 159 to about 201 of SEQ ID NO:2. In another embodiment, the MVEF protein has at least about 90 percent identity to amino acids from about 159 to about 201 of SEQ ID NO:2. In another embodiment, the MVEF protein has at least about 95 percent identity to amino acids from about 159 to about 201 of SEQ ID NO:2. In another embodiment, the MVEF protein has at least about 99 percent identity to amino acids from about 159 to about 201 of SEQ ID NO:2.

In embodiments, where the desired effect is an inhibition of cell proliferation, the MDA-7 variant is M1, M4 or M10.

Some embodiments of the invention are as follows:

M1 is an embodiment of MV1 having SEQ ID NO:3, and is a 159 amino acid peptide having a sequence as set forth from residues 48-206 of SEQ ID NO:2. The amino acid sequence of M1 is as follows: Gly Ala Gln Gly Gln Glu Phe His Phe Gly Pro Cys Gln Val Lys Gly Val Val Pro Gln Lys Leu Trp Glu Ala Phe Trp Ala Val Lys Asp Thr Met Gln Ala Gln Asp Asn Ile Thr Ser Ala Arg Leu Leu Gln Gln Glu Val Leu Gln Asn Val Ser Asp Ala Glu Ser Cys Tyr Leu Val His Thr Leu Leu Glu Phe Tyr Leu Lys Thr Val Phe Lys Asn Tyr His Asn Arg Thr Val Glu Val Arg Thr Leu Lys Ser Phe Ser Thr Leu Ala Asn Asn Phe Val Leu Ile Val Ser Gln Leu Gln Pro Ser Gln Glu Asn Glu Met Phe Ser Ile Arg Asp Ser Ala His Arg Arg Phe Leu Leu Phe Arg Arg Ala Phe Lys Gln Leu Asp Val Glu Ala Ala Leu Thr Lys Ala Leu Gly Glu Val Asp Ile Leu Leu Thr Trp Met Gln Lys Phe Tyr Lys Leu (SEQ ID NO:3).

M2 is an embodiment of MV2 having SEQ ID NO:4, and is a 144 amino acid peptide having a sequence as set forth from residues 63-206 of SEQ ID NO:2. The amino acid sequence of M2 is: Gly Val Val Pro Gln Lys Leu Trp Glu Ala Phe Trp Ala Val Lys Asp Thr Met Gln Ala Gln Asp Asn Ile Thr Ser Ala Arg Leu Leu Gln Gln Glu Val Leu Gln Asn Val Ser Asp Ala Glu Ser Cys Tyr Leu Val His Thr Leu Leu Glu Phe Tyr Leu Lys Thr Val Phe Lys Asn Tyr His Asn Arg Thr Val Glu Val Arg Thr Leu Lys Ser Phe Ser Thr Leu Ala Asn Asn Phe Val Leu Ile Val Ser Gln Leu Gln Pro Ser Gln Glu Asn Glu Met Phe Ser Ile Arg Asp Ser Ala His Arg Arg Phe Leu Leu Phe Arg Arg Ala Phe Lys Gln Leu Asp Val Glu Ala Ala Leu Thr Lys Ala Leu Gly Glu Val Asp Ile Leu Leu Thr Trp Met Gln Lys Phe Tyr Lys Leu (SEQ ID NO:4).

M3 is an embodiment of MV3 having SEQ ID NO:5, and is a 127 amino acid peptide having a sequence as set forth from residues 80-206 of SEQ ID NO:2. The amino acid sequence of M3 is: Gln Ala Gln Asp Asn Ile Thr Ser Ala Arg Leu Leu Gln Gln Glu ValLeu Gln Asn Val Ser Asp Ala Glu Ser Cys Tyr Leu Val His Thr LeuLeu Glu Phe Tyr Leu Lys Thr Val Phe Lys Asn Tyr His Asn Arg ThrVal Glu Val Arg Thr Leu Lys Ser Phe Ser Thr Leu Ala Asn Asn PheVal Leu Ile Val Ser Gln Leu Gln Pro Ser Gln Glu Asn Glu Met PheSer Ile Arg Asp Ser Ala His Arg Arg Phe Leu Leu Phe Arg Arg Ala Phe Lys Gln Leu Asp Val Glu Ala Ala Leu Thr Lys Ala Leu Gly GluVal Asp Ile Leu Leu Thr Trp Met Gln Lys Phe Tyr Lys Leu (SEQ ID NO:5).

M4 is an embodiment of MV4 having SEQ ID NO:6, and is a 103 amino acid peptide having a sequence as set forth from residues 104-206 of SEQ ID NO:2. The amino acid sequence of M4 is: Glu Ser Cys Tyr Leu Val His Thr Leu Leu Glu Phe Tyr Leu Lys Thr Val Phe Lys Asn Tyr His Asn Arg Thr Val Glu Val Arg Thr Leu Lys Ser Phe Ser Thr Leu Ala Asn Asn Phe Val Leu Ile Val Ser Gln Leu Gln Pro Ser Gln Glu Asn Glu Met Phe Ser Ile Arg Asp Ser Ala His Arg Arg Phe Leu Leu Phe Arg Arg Ala Phe Lys Gln Leu Asp Val Glu Ala Ala Leu Thr Lys Ala Leu Gly Glu Val Asp Ile Leu Leu Thr Trp Met Gln Lys Phe Tyr Lys Leu (SEQ ID NO:6).

M5 is an embodiment of MV5 having SEQ ID NO:7, and is a 76 amino acid peptide having a sequence as set forth from residues 131-206 of SEQ ID NO:2. The amino acid sequence of M5 is: Arg Thr Leu Lys Ser Phe Ser Thr Leu Ala Asn Asn Phe Val Leu Ile Val Ser Gln Leu Gln Pro Ser Gln Glu Asn Glu Met Phe Ser Ile Arg Asp Ser Ala His Arg Arg Phe Leu Leu Phe Arg Arg Ala Phe Lys Gln Leu Asp Val Glu Ala Ala Leu Thr Lys Ala Leu Gly Glu Val Asp Ile Leu Leu Thr Trp Met Gln Lys Phe Tyr Lys Leu (SEQ ID NO:7).

M6 is an embodiment of MV6 having SEQ ID NO:8, and is a 48 amino acid peptide having a sequence as set forth from residues 159-206 of SEQ ID NO:2. The amino acid sequence of M6 is: Met Phe Ser Ile Arg Asp Ser Ala His Arg Arg Phe Leu Leu Phe Arg Arg Ala Phe Lys Gln Leu Asp Val Glu Ala Ala Leu Thr Lys Ala Leu Gly Glu Val Asp Ile Leu Leu Thr Trp Met Gln Lys Phe Tyr Lys Leu (SEQ ID NO:8).

M7 is an embodiment of MV7 having SEQ ID NO:9, and is a 133 amino acid peptide having a sequence as set forth from residues 48-180 of SEQ ID NO:2. The amino acid sequence of M7 is: Gly Ala Gln Gly Gln Glu Phe His Phe Gly Pro Cys Gln Val Lys Gly Val Val Pro Gln Lys Leu Trp Glu Ala Phe Trp Ala Val Lys Asp Thr Met Gln Ala Gln Asp Asn Ile Thr Ser Ala Arg Leu Leu Gln Gln Glu Val Leu Gln Asn Val Ser Asp Ala Glu Ser Cys Tyr Leu Val His Thr Leu Leu Glu Phe Tyr Leu Lys Thr Val Phe Lys Asn Tyr His Asn Arg Thr Val Glu Val Arg Thr Leu Lys Ser Phe Ser Thr Leu Ala Asn Asn Phe Val Leu Ile Val Ser Gln Leu Gln Pro Ser Gln Glu Asn Glu Met Phe Ser Ile Arg Asp Ser Ala His Arg Arg Phe Leu Leu Phe Arg Arg Ala Phe Lys Gln Leu (SEQ ID NO:9).

M8 is an embodiment of MV8 having SEQ ID NO:10, and is a 11 amino acid peptide having a sequence as set forth from residues 48-158 of SEQ ID NO:2. The amino acid sequence of M8 is: Gly Ala Gln Gly Gln Glu Phe His Phe Gly Pro Cys Gln Val Lys Gly Val Val Pro Gln Lys Leu Trp Glu Ala Phe Trp Ala Val Lys Asp Thr Met Gln Ala Gln Asp Asn Ile Thr Ser Ala Arg Leu Leu Gln Gln Glu Val Leu Gln Asn Val Ser Asp Ala Glu Ser Cys Tyr Leu Val His Thr Leu Leu Glu Phe Tyr Leu Lys Thr Val Phe Lys Asn Tyr His Asn Arg Thr Val Glu Val Arg Thr Leu Lys Ser Phe Ser Thr Leu Ala Asn Asn Phe Val Leu Ile Val Ser Gln Leu Gln Pro Ser Gln Glu Asn Glu (SEQ ID NO:10).

M9 is an embodiment of MV9 having SEQ ID NO:11, and is a 83 amino acid peptide having a sequence as set forth from residues 48-130 of SEQ ID NO:2. The amino acid sequence of M9 is: Gly Ala Gln Gly Gln Glu Phe His Phe Gly Pro Cys Gln Val Lys Gly Val Val Pro Gln Lys Leu Trp Glu Ala Phe Trp Ala Val Lys Asp Thr Met Gln Ala Gln Asp Asn Ile Thr Ser Ala Arg Leu Leu Gln Gln Glu Val Leu Gln Asn Val Ser Asp Ala Glu Ser Cys Tyr Leu Val His Thr Leu Leu Glu Phe Tyr Leu Lys Thr Val Phe Lys Asn Tyr His Asn Arg Thr Val Glu (SEQ ID NO:11).

M10 is an embodiment of MV10 having SEQ ID NO:12, and is a 57 amino acid peptide having a sequence as set forth from residues 48-104 of SEQ ID NO:2. The amino acid sequence of M10 is: Gly Ala Gln Gly Gln Glu Phe His Phe Gly Pro Cys Gln Val Lys Gly Val Val Pro Gln Lys Leu Trp Glu Ala Phe Trp Ala Val Lys Asp Thr Met Gln Ala Gln Asp Asn Ile Thr Ser Ala Arg Leu Leu Gln Gln Glu Val Leu Gln Asn Val Ser Asp Ala Glu (SEQ ID NO:12).

Peptide MAB is an embodiment of MVAB having SEQ ID NO:13, and is a 39 amino acid peptide having a sequence as set forth from residues 63-101 of SEQ ID NO:2. The amino acid sequence of MAB is: Gly Val Val Pro Gln Lys Leu Trp Glu Ala Phe Trp Ala Val Lys Asp Thr Met Gln Ala Gln Asp Asn Ile Thr Ser Ala Arg Leu Leu Gln Gln Glu Val Leu Gln Asn Val Ser (SEQ ID NO:13).

Peptide MCD is an embodiment of MVCD having SEQ ID NO:14, and is a 50 amino acid peptide having a sequence as set forth from residues 105-154 of SEQ ID NO:2. The amino acid sequence of MCD is: Ser Cys Tyr Leu Val His Thr Leu Leu Glu Phe Tyr Leu Lys Thr Val Phe Lys Asn Tyr His Asn Arg Thr Val Glu Val Arg Thr Leu Lys Ser Phe Ser Thr Leu Ala Asn Asn Phe Val Leu Ile Val Ser Gln Leu Gln Pro Ser (SEQ ID NO:14).

Peptide EF is an embodiment of MVEF having SEQ ID NO:15, and is a 43 amino acid peptide having a sequence as set forth from residues 159-201 of SEQ ID NO:2. The amino acid sequence of MEF is: Met Phe Ser lie Arg Asp Ser Ala His Arg Arg Phe Leu Leu Phe Arg Arg Ala Phe Lys Gln Leu Asp Val Glu Ala Ala Leu Thr Lys Ala Leu Gly Glu Val Asp Ile Leu Leu Thr Trp Met Gln (SEQ ID NO:15).

A MDA-7 variant of the invention may comprise or be linked to a molecule that facilitates its biological activity. As a first example, such molecule may be a secretory signal peptide; where a nucleic acid encoding a MDA-7 variant is introduced into a cell, said secretory peptide would facilitate the secretion of the MDA-7 variant so as to produce a “bystander” effect (Su et al. Proc Natl Acad Sci USA. 2001; 98:10332-10337). The secretory peptide may be the secretory peptide of wild-type MDA-7 (i.e., residues 148), or another naturally occurring or synthetic secretory peptide e.g. cleavable signal peptide of human gamma-interferon (Colley et al. J Biol Chem. 1989, 264:17619-17622) or the NH2-terminal leader sequence of mouse immunoglobulin light chain precursor (Koren et al., Proc Natl Acad Sci USA. 1983, 80: 7205-7209). As a second example, the molecule may facilitate cell or tissue compartmentalization; e.g., the molecule may be a KDEL peptide that would favor retention of the variant in the endoplasmic reticulum, or the molecule may facilitate passage across a cell membrane, into the nucleus or through the blood brain barrier. As a third non-limiting example, utilization of the FFAT motif, a membrane targeting determinant found in several apparently unrelated lipid binding proteins (Loewen et al., EMBO J. 2003, 22: 2025-2035) may be used to facilitate targeting to the cell membrane. As a fourth non-limiting example, the 15-residue targeting motif of cAMP-dependent protein kinase anchoring protein (d-AKAPI) which targets proteins to either ER or mitochondria depending on interaction with each organelle (Ma and Taylor, J Biol Chem. 2002, 277: 27328-27336) may be used for targeting to both these organelles simultaneously.

Proteins targeted to the ER by a secretory leader sequence can be released into the extracellular space as a secreted protein. For example, vesicles containing secreted proteins can fuse with the cell membrane and release their contents into the extracellular space—a process called exocytosis. Exocytosis can occur constitutively or after receipt of a triggering signal. In the latter case, the proteins may be stored in secretory vesicles (or secretory granules) until exocytosis is triggered. Similarly, proteins residing on the cell membrane can also be secreted into the extracellular space by proteolytic cleavage of a “linker” holding the protein to the membrane.

A MDA-7 variant of the invention may comprise elements or be linked to elements that improve its stability or activity. These modifications include but are not limited to N-terminal acetylation or C-terminal amidation, incorporation of D-amino acids or unnatural amino acids including but not limited to β-alanine, ornithine, hydroxyproline; or substitution at the peptide termini with biotin or long chain alkanes; addition of certain side chain modifications including but not limited to phosphorylation of serine, threonine or tyrosine residues; cyclisation via intramolecular disulphide bond formation; and formation of cyclic amides or radioconjugates. Stabilization of the peptide or protein may be further achieved by, as non-limiting examples, utilization of matrices that enhance delivery, increase stability or achieve controlled release rate such as natural and synthetic biopolymers and cell responsive matrices (Zisch et al., 2003, Cardiovasc Pathol 12: 295-310), or alginate microcapsules (Schneider et al., 2003, J Microencapsul 20:627-636).

The MDA-7 variants of the invention may be produced by any method known in the art. Such methods include but are not limited to chemical synthesis and recombinant DNA techniques.

The terms “nucleic acid molecule,” “nucleotide,” “polynucleotide,” and “nucleic acid” are used interchangeably herein to refer to polymeric forms of nucleotides of any length. They can include both double- and single-stranded sequences and include, but are not limited to, cDNA from viral, prokaryotic, and eucaryotic sources; mRNA; genomic DNA sequences from viral (e.g. DNA viruses and retroviruses) or prokaryotic sources, RNAi, cRNA, anti-sense molecules, ribozymes and synthetic DNA sequences. The term also captures sequences that include any of the known base analogs of DNA and RNA.

“Operably linked” refers to an arrangement of elements wherein the; components so described are configured so as to perform their desired function. Thus, a given promoter operably linked to a coding sequence is capable of effecting the expression of the coding sequence when the proper transcription factors, etc., are present. The promoter need not be contiguous with the coding sequence, so long as it functions to direct the expression thereof. Thus, for example, intervening untranslated yet transcribed sequences can be present between the promoter sequence and the coding sequence, as can translated introns, and the promoter sequence can still be considered “operably linked” to the coding sequence.

With regard to production of MDA-7 variants using recombinant DNA techniques, the invention provides for nucleic acids encoding said variants. Such nucleic acids may either be nucleic acid fragments of the aforelisted mda-7 nucleic acids encoding the variants, or may be nucleic acids designed, using the genetic code, to encode such variants.

For example, but not by limitation, M1 is encoded by a nucleic acid having SEQ ID NO:16, M2 is encoded by a nucleic acid having SEQ ID NO:17, M3 is encoded by a nucleic acid having SEQ ID NO:18, M4 is encoded by a nucleic acid having SEQ ID NO:19, M5 is encoded by a nucleic acid having SEQ ID NO:20, M6 is encoded by a nucleic acid having SEQ ID NO:21, M7 is encoded by a nucleic acid having SEQ ID NO:22, M8 is encoded by a nucleic acid having SEQ ID NO:23, M9 is encoded by a nucleic acid having SEQ ID NO:24, M10 is encoded by a nucleic acid having SEQ ID NO:25, AB domain is encoded by a nucleic acid having SEQ ID NO:26, CD domain is encoded by a nucleic acid having SEQ ID NO:27 and EF domain is encoded by a nucleic acid having SEQ ID NO:28.

A nucleic acid encoding a MDA-7 variant of the invention may be comprised in a suitable vector molecule, and may optionally be operatively linked to a suitable promoter element, for example, but not limited to, the cytomegalovirus immediate early promoter, the Rous sarcoma virus long terminal repeat promoter, the human elongation factor 1α promoter, the human ubiquitin c promoter, etc.. It may be desirable, in certain embodiments of the invention, to use an inducible promoter. Non-limiting examples of inducible promoters include the murine mammary tumor virus promoter (inducible with dexamethasone); commercially available tetracycline-responsive or ecdysone-inducible promoters, etc. In non-limiting embodiments of the invention, the promoter may be selectively active in cancer cells; one example of such a promoter is the PEG-3 promoter, as described in International Patent Application No. PCT/US99/07199, Publication No. WO 99/49898 by Fisher et al., published on Oct. 7, 1999; other non-limiting examples include the prostate specific antigen gene promoter (O'Keefe et al., 2000, Prostate 45:149-157), the kallikrein 2 gene promoter (Xie et al., 2001, Human Gene Ther. 12:549-561), the human alpha-fetoprotein gene promoter (Ido et al., 1995, Cancer Res. 55:3105-3109), the c-erbB-2 gene promoter (Takakuwa et al., 1997, Jpn. J. Cancer Res. 88:166-175), the human carcinoembryonic antigen gene promoter (Lan et al., 1996, Gastroenterol. 111: 1241-1251), the gastrin-releasing peptide gene promoter (Inase et al., 2000, Int. J. Cancer 85:716-719), the human telomerase reverse transcriptase gene promoter (Pan and Koenman, 1999, Med. Hypotheses 53:130-135), the hexokinase II gene promoter (Katabi et al., 1999, Human Gene Ther. 10:155-164), the L-plastin gene promoter (Peng et al., 2001, Cancer Res. 61:4405-4413), the neuron-specific enolase gene promoter (Tanaka et al., 2001, Anticancer Res. 21:291-294), the rnidkine gene promoter (Adachi et al., 2000, Cancer Res. 60:4305-4310), the human mucin gene MUC1 promoter (Stackhouse et al., 1999, Cancer Gene Ther. 6:209-219), and the human mucin gene MUC4 promoter (Genbank Accession No. AF241535), which is particularly active in pancreatic cancer cells (Perrais et al., J Biol Chem. 2001, 276:30923-30933).

Suitable expression vectors include virus-based vectors and non-virus based DNA or RNA delivery systems. Examples of appropriate virus-based gene transfer vectors include, but are not limited to, pCEP4 and pREP4 vectors from Invitrogen, and, more generally, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6:113-138; Connelly, 1999, Curr. Opin. Mol. Ther. 1:565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996,Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

Non-limiting examples of non-virus-based delivery systems which may be used according to the invention include, but are not limited to, so-called naked nucleic acids (Wolff et al., 1990, Science 247:1465-1468), nucleic acids encapsulated in liposomes (Nicolau et al., 1987, Methods in Enzymology 198:157-176), nucleic acid/lipid complexes (Legendre and Szoka, 1992, Pharmaceutical Research 9:1235-1242), and nucleic acid/protein complexes (Wu and Wu, 1991, Biother. 3:87-95). MDA-7 may also be produced by yeast or bacterial expression systems. For example, bacterial expression may be achieved using plasmids such as pGEX expression system (Amersham Biosciences, Piscataway, N.J.), pQE His-tagged expression system (Qiagen, Valencia, Calif.), pET His-tagged expression system (EMD Biosciences, Inc., La Jolla, Calif.), or IMPACT expression system (New England Biolabs, Beverly, Mass.).

Depending on the expression system used, nucleic acid may be introduced by any standard technique, including transfection, transduction, electroporation, bioballistics, microinjection, etc.

In non-limiting embodiments of the invention, the expression vector is an E1-deleted human adenovirus vector of serotype 5. To prepare such a vector, an expression cassette comprising a transcriptional promoter element operatively linked to a MDA-7 variant coding region and a polyadenylation signal sequence may be inserted into the multiple cloning region of an adenovirus vector shuttle plasmid, for example pXCJL.1 (Berkner, 1988, Biotechniques 6:616-624). In the context of this plasmid, the expression cassette may be inserted into the DNA sequence homologous to the 5′ end of the genome of the human serotype 5 adenovirus, disrupting the adenovirus E1 gene region. Transfection of this shuttle plasmid into the E1-transcomplementing 293 cell line (Graham et al., 1977, J. General Virology 36:59-74), or another suitable cell line known in the art, in combination with either an adenovirus vector helper plasmid such as pJM17 (Berkner, 1988, Biotechniques 6:616-624; McGrory et al., 1988, Virology 163:614-617) or pBHG10 (Bett et al., 1994, Proc. Natl. Acad. Sci. U.S.A. 91: 8802-8806) or a ClaI-digested fragment isolated from the adenovirus 5 genome (Berkner, 1988, Biotechniques 6:616-624), allows recombination to occur between homologous adenovirus sequences contained in the adenovirus shuttle plasmid and either the helper plasmid or the adenovirus genomic fragment. This recombination event gives rise to a recombinant adenovirus genome in which the cassette for the expression of the foreign gene has been inserted in place of a functional E1 gene. When transcomplemented by the protein products of the human adenovirus type 5 E1 gene (for example, as expressed in 293 cells), these recombinant adenovirus vector genomes can replicate and be packaged into fully-infectious adenovirus particles. The recombinant vector can then be isolated from contaminating virus particles by one or more rounds of plaque purification (Berkner, 1988, Biotechniques 6:616-624), and the vector can be further purified and concentrated by density ultracentrifugation.

In a non-limiting embodiment of the invention, a nucleic acid encoding a MDA-7 variant, in expressible form, may be inserted into the modified Ad expression vector pAd.CMV (Falck-Pedersen et al., 1994, Mol. Pharmacol. 45:684-689). This vector contains, in order, the first 355 base pairs from the left end of the adenovirus genome, the cytomegalovirus immediate early promoter, DNA encoding splice donor and acceptor sites, a cloning site for the mda-7 variant gene, DNA encoding a polyadenylation signal sequence from the globin gene, and approximately three kilobase pairs of adenovirus sequence extending from within the E1B coding region. This construct may then be introduced into 293 cells (Graham et al., 1977, J. Gen. Virol. 36:59-72) together with plasmid JM17 (above), such that, as explained above, homologous recombination can generate a replication defective adenovirus containing MDA-7 variant encoding nucleic acid.

The invention provides a method of producing polypeptide by providing an isolated nucleic acid of the invention and expressing it in an expression system to produce the polypeptide. Both cell-based and cell-free expression systems can be used to practice the method. Both prokaryotic and eukaryotic expression systems are suitable. For example, the expression system may comprise a host cell transfected with an isolated nucleic acid molecule of the invention, forming a recombinant host cell, which can be cultured. Cell-free expression systems suitable for practicing the method include wheat germ lysate expression systems, rabbit reticulocyte expression systems, ribosomal displays, and E. coli lysate expression systems. The invention provides a polypeptide produced by both cell-based and cell-free expression systems. It provides a polypeptide produced by these systems with mammalian, insect, plant, yeast, or bacterial host cells.

The invention provides for an antibody, or antigen-binding fragment thereof, that binds to the polypeptide of SEQ ID NO:3, 4, 5, 6, 7, 8, 9, 10, 11, 12, 13, or 14.

The invention also provides for peptidomimetic compounds that are structurally similar to the MDA-7 variants provided by the invention. Generally, a peptidomimetic of a compound X refers to a compound in which chemical structures of X necessary for functional activity of X have been replaced with other chemical structures that mimic the conformation of X. Peptidomimetics are currently exploited to overcome problems associated with their parent peptides. Improvements provided by the peptidomemtic over the parent peptide include increased selectivity, oral bioavailability and prolonging the activity by hindering enzymatic degradation within the organism. Peptidomimetics can include organic compounds and modified peptides that mimic the three-dimensional shape of a parent peptide. Examples of peptidomimetics include MDA-7 variants of the invention comprising a peptide portion in which the peptide backbone is substituted with one or more benzodiazepine molecules (see e.g., James, G. L. et al., (1993) Science 260:1937-1942).

Assays To Confirm MDA-7 Variant Activity

A MDA-7 variant of the invention may be tested for activity in modulating cell proliferation and/or differentiation.

“Modulating cell proliferation” includes promoting or inhibiting proliferation in general as well as under particular conditions such as, for example, colony formation in monolayer or soft agar. The effect of a MDA-7 variant on proliferation may be evaluated by measuring the rate at which a population of cells proliferate (e.g., the doubling time) or by measuring the percentage of cells in mitosis (e.g., the number of cells in metaphase). In one embodiment, the MDA-7 variant modulates proliferation by at least about 5, 10, 20, 30, 40, or 50 percent. “Slight” activity as defined herein refers to modulation of about 5-15 percent.

The modulatory activity of a MDA-7 variant may be tested by either introducing a nucleic acid encoding the variant, in expressible form, into a test cell, for example by transfection or by transduction using a viral vector, as set forth in the preceding section. Suitable test cells include cells whose proliferation is modulated by native MDA-7, including malignant cells such as, but not limited to, the cell lines used in the working examples described below. Alternatively, the modulatory activity of a MDA-7 variant may be tested by exposing a test cell to an effective concentration of variant polypeptide. An effective concentration of the protein or peptide may be in the range of 18 to 50 ng per microliter.

In an embodiment of the invention, the ability of a MDA-7 variant to modulate cell proliferation may be assayed as follows. The proliferation rate may be determined by ability of cells to form colonies on 6 cm tissue culture dishes, 2-3 weeks after treatment with MDA-7 variants and respective controls. Herein, effect on cell proliferation in the presence or absence of a growth inhibitory or apoptosis inducing substance or molecule is measured by the ability of cells to grow and divide to form foci of >50 cells/colony. This is an indirect measure of cell survival and is determined relative to colony numbers formed by a similar number of cells of the same cell type, comparatively measured in the absence of the inhibitor or some other related neutral control substance or molecule. Approximately 5×10³ to 5×10⁴ cells may be plated and allowed to attach in appropriate growth medium before treatment with MDA-7 variants by DNA transfection or infection with appropriate viral vector or purified protein. Surviving cells may be scored as visible colonies after incubation in presence or absence of a selective drug, for example after, 2-3 weeks. The resultant colonies (comprising foci of >50 cells/ colony) may be visualized by staining plates with Giemsa dye (Su et al., 1998, Proc. Natl. Acad. Sci USA, 95: 14400-14405).

Use of a MDA-7 Variant as a Gene Therapy

A MDA-7 variant may be used to modulate cell proliferation in a subject, wherein a nucleic acid encoding the variant, in expressible form, may be introduced into a cell of the subject.

In non-limiting embodiments, the nucleic acid encoding the MDA-7 variant may be contained in a viral vector, operably linked to a promoter element that is inducible or constitutively active in the target cell. In non-limiting embodiments, the viral vector is a replication-defective adenovirus. In non-limiting embodiments, the viral vector is selected from the group consisting of retrovirus, adenovirus, adeno-associated virus, vaccinia virus, herpesvirus and polyoma virus.

In a non-limiting embodiment of the invention, a viral vector containing a nucleic acid encoding a MDA-7 variant, such as an MVX polypeptide operably linked to a suitable promoter element, may be administered to a population of target cells at a multiplicity of infection (MOI) ranging from 10-100 MOI.

In another non-limiting embodiment, the amount of a viral vector administered to a subject may be 1×10⁹ pfu to 1×10¹² pfu.

In non-limiting embodiments, a nucleic acid encoding a MDA-7 variant, comprised in a vector or otherwise, may be introduced into a cell ex vivo and then the cell may be introduced into a subject. For example, a nucleic acid encoding a MDA-7 variant may be introduced into a cell of a subject (for example, an irradiated tumor cell, glial cell or fibroblast) ex vivo and then the cell containing the nucleic acid may be optionally propagated and then (with its progeny) introduced into the subject.

Use of a MDA-7 Variant as a Peptide Therapy

Alternatively, a MDA-7 variant may be used in polypeptide therapy of a subject in need of such treatment. As such, the MDA-7 variant of the invention may be prepared by chemical synthesis or recombinant DNA techniques, purified by methods known in the art, and then administered to a subject in need of such treatment. MDA-7 variant may be comprised, for example, in solution, in suspension, and/or in a carrier particle such as microparticles, liposomes, or other protein-stabilizing formulations known in the art. In a non-limiting example, formulations of MDA-7 variant peptides may stabilized by addition of zinc and/or protamine stabilizers as in the case of certain types of insulin formulations.

The invention provides both nucleic acid and polypeptide compositions, each comprising a carrier. They may, for example be provided as vector compositions, and/or host cell compositions. The carrier may be a pharmaceutically acceptable carrier or an excipient. In non-limiting embodiments, a MDA-7 variant may be linked covalently or non-covalently, to a carrier protein. In an embodiment of the invention, the carrier protein is non-immunogenic.

In non-limiting embodiments, a MDA-7 variant polypeptide is administered in an amount which achieves a local concentration in the range of 18 to 50 ng per microliter. For example, a subject may be administered a range of 50-100 mg per kilogram. For a human subject, the dose range may be between 1000-2500 mg/day.

Combined Therapy Using MDA-7 Protein Variants

The invention further encompasses the use of MDA-7 variants in combination with other forms of therapy. For example, it encompasses the use of MDA-7 variants in combination with other agents that have an anti-proliferative effect, including, but not limited to, radiation therapy and chemotherapeutic agents.

As a first non-limiting example, a MDA-7 variant may be administered together with a generator of free radicals (International Patent Application No. PCT/US03/28512, by Fisher et al., published as WO 04/060269 on Jul. 22, 2004 by the Trustees of Columbia University and Virginia Commonwealth University). Examples of free radical generators include, but are not limited to arsenic trioxide, NSC656240, 4-HPR, and cisplatin. Examples of ROS include but are not limited to singlet oxygen, hydrogen peroxide, superoxide anion, hydroxyl radicals, peroxynitrite, and oxidants. In other embodiments, the free radical generators are arsenic trioxide, NSC656240 or 4-HPR. In other embodiments, the disruptor of mitochondrial membrane potential is PK 11195.

As a second non-limiting example, a MDA-7 variant may be administered together with a regimen of radiation therapy (International Patent Application No. PCT/US03/28512, by Fisher et al., published as WO 04/060269 on Jul. 22, 2004 by the Trustees of Columbia University). In non-limiting embodiments, a MDA-7 variant may be administered together with between 2 and 100 Gy of radiation, either as a single treatment or in multiple treatments. In one non-limiting embodiment of the invention, one external treatment of 2 Gy may be administered each of 5 days a week for six weeks for a total of 60 Gy. If intraoperative radiation is administered, the amount administered may be between 3 and 15 Gy total. In one embodiment, the amount of radiation administered is around 6 Gy.

As a third non-limiting embodiment, a MDA-7 variant may be administered together with an anti-ras agent (International Patent Application No. PCT/US02/26454, by Fisher et al., published as WO 03/016499 on Feb. 27, 2003 by the Trustees of Columbia University); particularly in the treatment of a disorder of cell proliferation associated with a mutation in a ras gene. Suitable anti-ras agents include, but are not limited to, small interfering RNAs (RNAi), antisense RNA (including but not limited to oligonucleotides having phosphorothioate residues), or farnesyl transferase inhibitors.

As a fourth non-limiting embodiment, a MDA-7 variant may be administered together with a chemotherapy agent, including, but not limited to, interferon alpha, tamoxifen, cisplatin, daunorubicin, carmustine, dacarbazine, etoposide, fluorouracil, ifosfamide, methotrexate, mitomycin, mitoxanthrone HCl, vincristine, vinblastine, and adriamycin, to name a few.

As a fifth non-limiting embodiment, a MDA-7 variant may be administered together with an anti-cancer antibody, such as, but not limited to, trastuzumab (Herceptin).

Further, a MDA-7 variant may be administered together with more than one other anti-proliferative agent (e.g., free radical generator, radiation, anti-ras agent, chemotherapeutic agent, anticancer antibody, etc.).

The amounts of anti-proliferative therapy added to the dose of MDA-7 may be those doses conventionally used for such therapy. Alternatively, the combination of MDA-7 with another form of antiproliferative therapy may allow for the use of lower doses of said antiproliferative therapy.

Conditions Which May Be Treated

The invention, in particular non-limiting embodiments, provides for the treatment of disorders characterized by excessive cell proliferation. Such disorders include, but are not limited to, non-malignant conditions, including but not limited to psoriasis, keratoacanthoma, polycythemia, non-neoplastic recurrent nodular goiter, subglottic cysts, capillary hemangioma, benign osteoma, uterine leiomyomas and other non-malignant neoplasms or recurrent cysts, and malignant conditions including but not limited to, cancers of the skin, such as basal cell carcinoma, squamous cell carcinoma and melanoma; cancers of the nervous system such as glioblastoma, astrocytoma, and oligodendroma; cancers of the bone such as osteosarcoma; leukemias; lymphomas; breast cancer; ovarian cancer; prostate cancer; testicular cancer; bladder cancer; cancers of the gastrointestinal system such as gastric cancer, duodenal cancer, colon and rectal cancer; hepatocellular carcinoma, carcinoma of the pancreas, carcinoma of the gall bladder, adrenal cancer, renal cell carcinoma, and cancers of the lung such as small cell and non-small cell carcinoma and mesothelioma.

The invention provides for the treatment of a proliferative disease, such as a mammary adenocarcinoma, non-small cell lung carcinoma, breast tumors, lung tumors, prostate tumors, colon tumors, stomach tumors, bladder tumors, glioblastomas, and/or skin cancer.

In another non-limiting embodiment, the invention provides a means to restrict or limit inflammatory disease by inhibiting the activity of mda-7 and other inflammatory cytokines such as IL-10 and IL-20. Such disorders include, but are not limited to, inflammatory bowel disease, chronic asthma and other pulmonary inflammatory diseases, inflammatory neurodegenerative disorders, cutaneous T-cell lymphomas, rheumatoid arthritis, psoriasis etc., whose pathologies involve the activity of pro-inflammatory cytokines, the inhibition of which could result in alleviation of symptoms.

### EXAMPLES

### Example 1

### Construction of mda-7 Deletion Expression Vectors and Expression of mda-7 Deletion Mutants in Cells

Materials and Methods:

Human cancer cell lines and cell culture: Human cervical carcinoma (HeLa) and prostate carcinoma (DU-145) derived cell lines were obtained from the ATCC (Manassas, Va.) and grown in Dulbecco's modification of Eagle's medium supplemented with 10% fetal calf serum and maintained in an cell culture incubator at 37° C. with 5% CO₂ atmosphere and 100% humidity. Cells were selected with 50 μg/ml hygromycin where applicable, e.g. after transfection with the pREP4 vector or other constructs cloned in the pREP4 vector. Transfection to introduce plasmid DNA into cells for gene expression was performed utilizing Lipofectamine 2000 reagent according to conditions recommended by the manufacturer (Invitrogen, Carlsbad, Calif.).

Monolayer growth and colony formation assay: To study the effect of various constructs, transfection of these was performed using Lipofectamine 2000 reagent with the pREP4 vector (Invitrogen, Carlsbad, Calif.) as control or with pREP4 into which specific domains of mda-7 had been cloned. Around 5×10³ to 5×10⁴ cells were plated and allowed to attach in appropriate growth medium before transfection with pREP4 plasmid DNA containing no insert, full-length wild-type or variant MDA-7, followed by incubation for 2-3 weeks while applying selection with 50 μg/ml hygromycin, to inhibit growth of non-transfected cells. After colony formation, plates were stained with Geimsa stain and colonies containing >50 cells/colony were scored as previously described (Su et al., 1998, Proc. Natl. Acad. Sci USA, 95: 14400-14405). Transfection was performed in triplicate sets to obtain statistically valid colony counts for each transfection.

Construction of mda-7 deletion expression vectors: The variant forms of full length MDA-7 (constructs M1 to M10, MAB, MCD and MEF) were constructed utilizing PCR with specific primers whose sequence defined and delimited regions encompassing the specific amino acid coordinates of MDA-7 as described above. Essential regulatory signals including addition of an initiator methionine codon at the start of each open reading frame and translational stop codon at the end was also introduced by means of PCR by their incorporation into the specific primers used to construct the variants, where necessary. Other transcriptional regulatory sequences that drove transcription of plasmid in transfected cells, including the promoter and polyadenylation site were contained in the pREP4 vector sequence abutting the plasmid multiple cloning sites utilized to clone specific MDA-7 sequences generated by PCR. All constructs were sequence verified to confirm the deletion encoded as well as integrity of the open reading frame to be expressed.

Results:

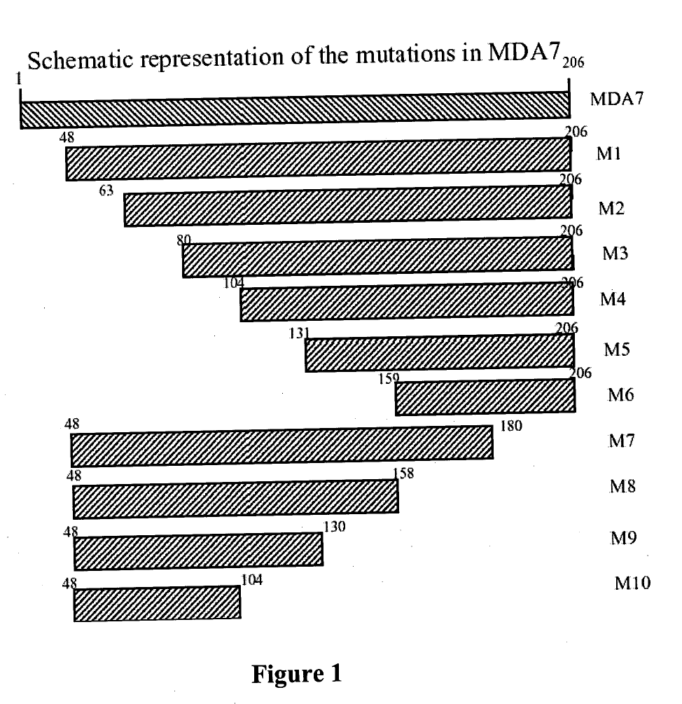

Using a PCR based strategy, ten distinct deletion constructs encompassing amino acid coordinates of the MDA-7 protein including 48-206 (M1), 63-206 (M2), 80-206 (M3), 104-206 (M4), 131-206 (M5) and 159-206 (M6) making up a set of amino-terminal deletions and encompassing amino acid coordinates 48-180 (M7), 48-158 (M8), 48-130 (M9) and 48-104 (M10) making up a set of carboxyl-terminal deletions were cloned into the mammalian expression vector pREP4 (Invitrogen, Carlsbad, Calif.). This vector system has been previously utilized to analyze growth suppressive effects of the mda-7 gene (Jiang et al., 1996, Proc Natl Acad Sci USA. 93:9160-9165). Deletions were constructed semi-randomly at approximately 20 amino acid intervals between positions 48-159 from the N-terminal and between positions 104 to 180 from the C-terminus (FIG. 1).

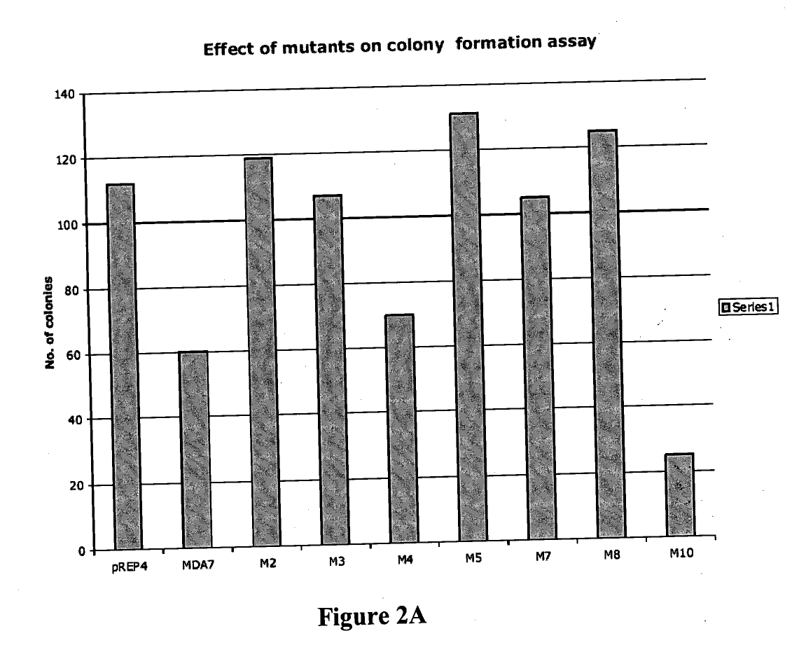

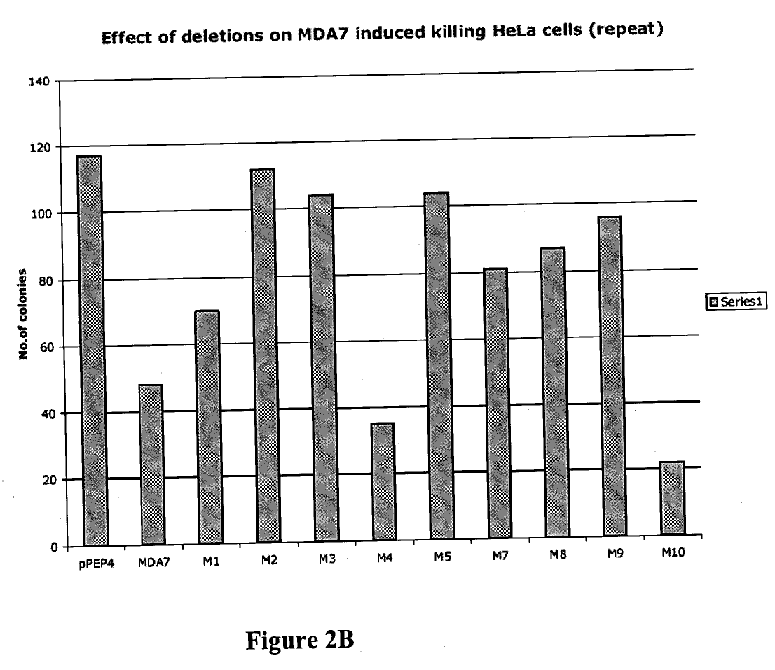

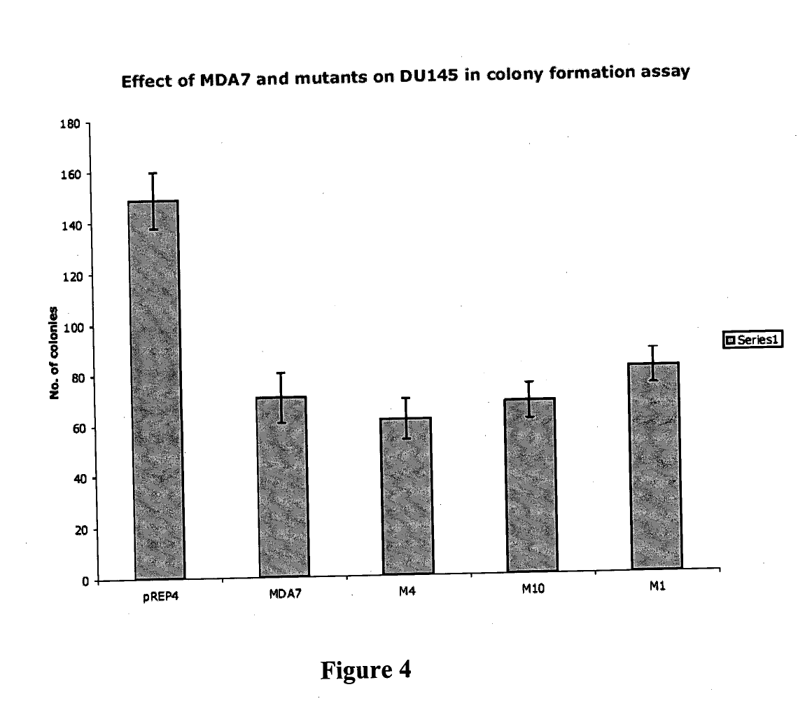

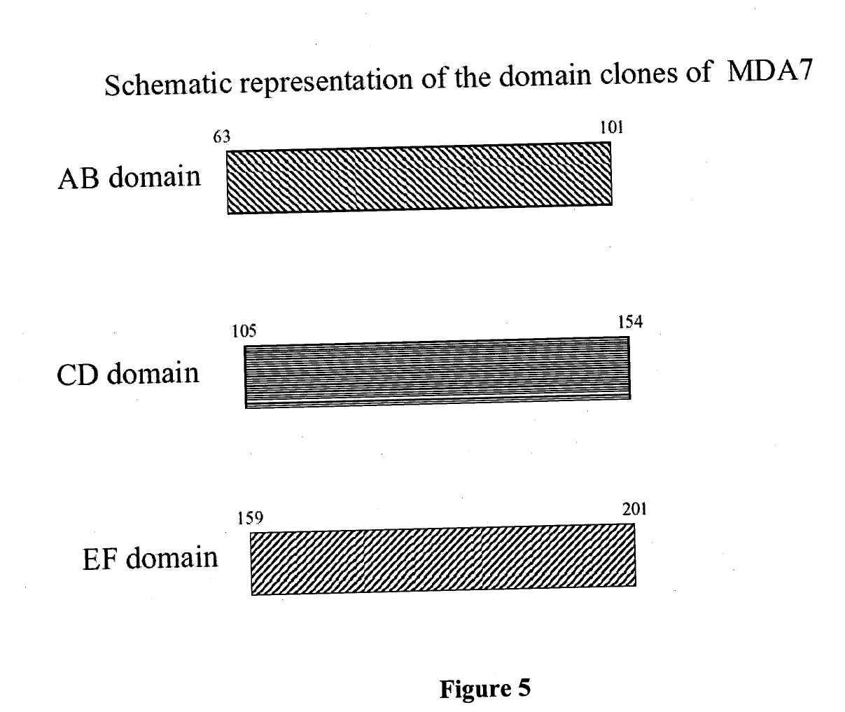

The phenotypic effect of expressing individual mutant constructs was initially analyzed in HeLa (human cervical cancer) cells due to their high transformation efficiency and relatively high susceptibility to mda-7 induced apoptosis (Jiang et al., 1996, Proc Natl Acad Sci USA. 93:9160-9165). Transfected cells were plated at densities of between 5×10³ to 5×10⁴ cells per dish and transfected with respective expression vectors. Controls included empty pREP4 vector or pREP4 encoding the entire mda-7 reading frame (FIGS. 2A and 2B, pREP4 and MDA7 bars). The effect on cell survival after exposure to different mda-7 deletion mutants was scored as average number of colonies formed (comprising of >50 cells/colony) and derived from average values of colony numbers from 3 plates per construct. While the negative control, pREP4, gave an average of 110 colonies, full-length mda-7 expressing plates gave an average of 60 colonies/plate. By contrast, cells expressing M2, M3, M5, M7, M8 and M9 had minimum to no effect on colony formation compared to pREP4 control. Constructs M1, M4 and M10 however had significant growth inhibitory effects forming average colony numbers of 60 for M1, 50 for M4 and 20 for M10 respectively. Therefore M1, M4 and M10 showed the ability to partially or fully reproduce the growth inhibitory effect of wild-type MDA-7. The remaining constructs tested displayed slight or no apparent influence on cell growth and survival when over expressed in HeLa cells (FIGS. 2A and 2B). When the experiment was repeated in DU-145 (human prostate cancer) cells, all three constructs, M1, M4 and M10 were statistically comparable in inhibitory activity to full length MDA-7, showing an average of 50% inhibition in colony formation compared to pREP4 vector (60 versus 150 average number of colonies; FIG. 4). These series of experiments with deletion variants of mda-7, in particular M1, M4 and M10 indicate that specific regions to MDA-7, when expressed in isolation, can retain the capacity of cancer cell killing hitherto only demonstrated by the intact native molecule.

Three additional internal deletion variants were constructed that approximated the major helical regions of the MDA-7 molecule. Thus the AB-, CD- and EF-domain constructs were delineated based on a helical overlay or threading of the known IL-10 crystal structure onto the predicted structure of MDA-7 since both belong to the four-helix bundle family of cytokines (Pestka et al., Annu Rev Immunol. 2004, 22:929-979). The basis of constructing these variants was to follow, at least partially, a rational structure-based framework of mutagenesis since helical regions have been shown to be important for cytokine activity of the four-helix bundle cytokines (Pestka et al., Annu Rev Immunol. 2004, 22:929-979). Expression constructs were made by cloning PCR generated molecules into the pREP4 vector and these were tested in colony formation assays as with the previous series of variants (M1 to M10).

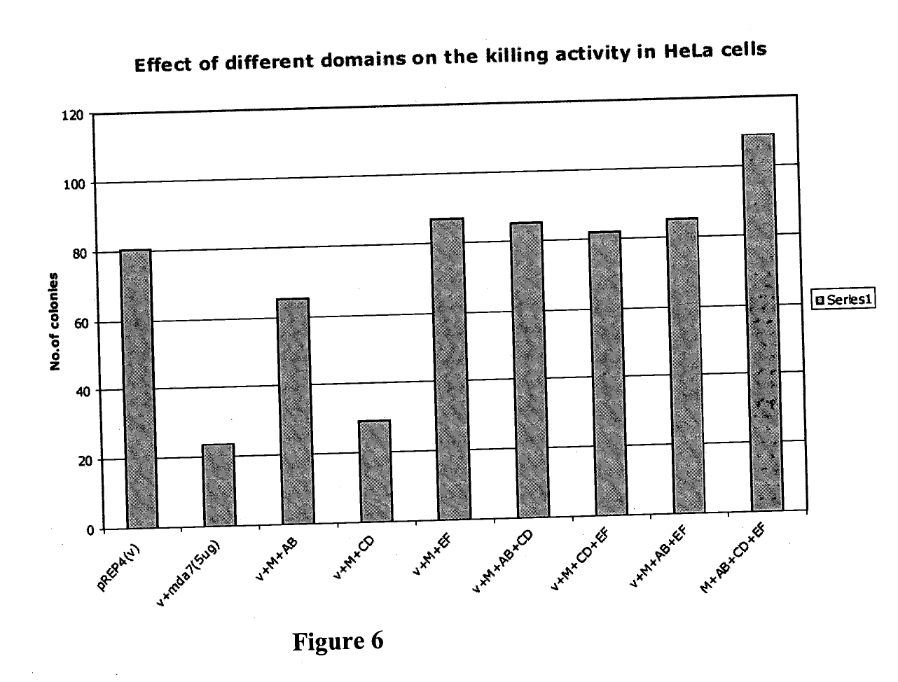

Transfection experiments utilizing the AB, CD and EF-series variants were variations of those performed using the M1-M10 series, in that each construct was co-transfected with vector (v), wild-type full length expressing construct (M) and in combinations with each other (FIG. 6) as opposed to transfection alone with the M1-M10 series. The total amount of input DNA per transfection was maintained by adjusting the concentrations of vector, MDA-7 and deletion construct DNAs to ensure that the extent of inhibition was comparable between different points. This experimental design permitted the determination of interference or synergy of activity of mutants with the wild-type molecule or with each other.

In HeLa cells, AB-domain (“MAB”), when co-transfected with full length MDA-7, was able to reverse the growth inhibiting ability of MDA-7 (FIG. 6, v+M+AB bar), as did v+M+EF, v+M+AB+CD, v+M+BC+EF, v+M+AB+EF, v+AB+CD+EF. However v+M+CD did not have any influence on the inhibitory activity of full length MDA-7.

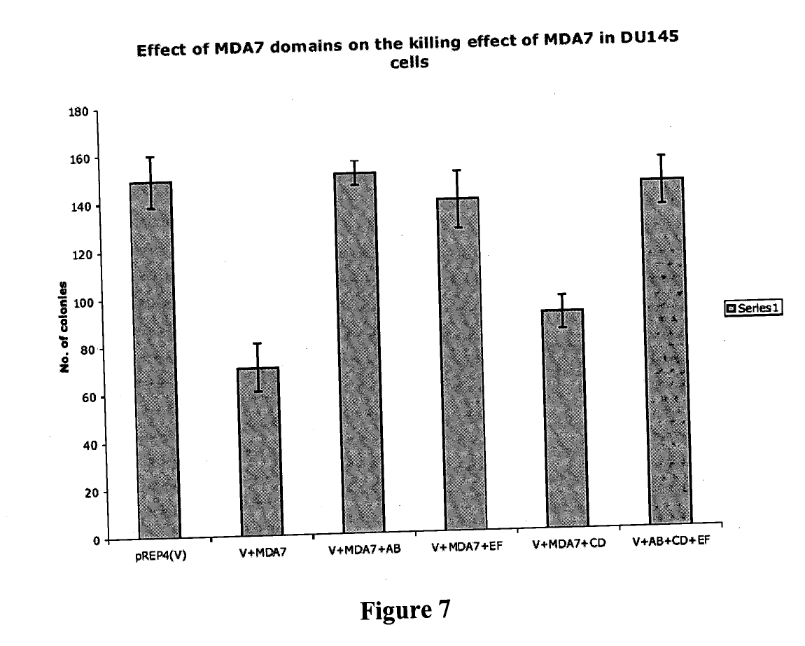

When a similar series of experiments was performed in DU-145 cells a similar pattern of inhibition was observed (FIG. 7). From these results it appears that at least certain partial helical domains of MDA-7 (i.e., the AB and EF domains), when co-expressed with the wild-type molecule, are able to reverse the growth inhibitory properties of the wild-type through currently unknown mechanisms. These might involve protein-protein interactions between full-length MDA-7, co-expressed variants and other cellular mediators of MDA-7 activity that might be distributed between active full-length MDA-7 containing complexes and inactive variant associated complexes.

The results obtained with constructs M1, M4 and M10 demonstrate that specific regions of the MDA-7 polypeptide are able to induce apoptosis in transformed cells. The M1 peptide corresponds to the entire active region of MDA-7, less the first 46 amino acids comprising the majority of N-terminal signal sequence. Removal of this sequence has been shown to impair the ability of cells to secrete MDA-7 protein. However, recent studies have demonstrated that this truncated molecule (lacking the secretory peptide) has the capacity to induce transformed cell specific apoptosis by localizing in the endoplasmic reticulum and likely induces apoptosis via the unfolded protein response as has been previously demonstrated indirectly for the wild-type molecule (Sauane et al., Cancer Res. 2004, 64: 2988-2993). The two non-overlapping constructs M4 and M10 still retain apoptosis inducing activity individually, despite being truncated forms of the MDA-7 peptide as well as not having a common region to which this activity may be attributed.

### Example 2

### Bip/GRP78 is an Intracellular Target for MDA-7/IL-24 Induction of Cancer-specific Apoptosis

Mda-7/IL-24 is a unique member of the IL-10 gene family that induces cancer-selective growth suppression and apoptosis in a wide spectrum of human cancers in cell culture, animal models and in clinical trials. Using deletion analysis, a specific mutant of MDA-7/IL-24, M4, consisting of amino acids 104 to 206 is described that retains the cancer-specific growth suppressive and apoptosis-inducting properties of the full-length protein. MDA-7/IL-24 and M4 physically interact with BiP/GRP78, localize in the endoplasmic reticulum and activate p38 MAPK and GADD gene expression culminating in apoptosis. These studies present novel insights into the mechanism of action of MDA-7/IL-24 and provide an opportunity to develop improved therapeutic versions of this cancer-specific apoptosis-inducing cytokine.

Mda-7/IL-24 has considerable potential for cancer gene therapy, recently validated in patients. Novel insights are provided into the mechanism of action of this cancer-specific apoptosis-inducing cytokine gene, identifying a specific deletion mutant M4 containing ˜50% of the full-length protein that retains the properties of the unmodified MDA-7/IL-24 protein. Rationally designed mutational analysis indicates the importance of specific regions in the C and F helices of MDA-7/IL-24 and interactions with BiP/GRP78 in mediating cancer-selective killing properties. These findings elucidate new targets and approaches that can be used to develop improved applications of this novel cytokine for cancer gene therapy. Mda-71IL-24 is an intriguing multifunctional gene product that exhibits considerable potential as a gene therapy for cancer (Fisher et al., 2003; Fisher, 2005; Gupta et al., 2005; Lebedeva et al., 2005a). When administered by means of a replication incompetent adenovirus (Ad.mda-7), growth suppression and apoptosis are induced in a broad spectrum of tumor cells both in vitro and in vivo in human tumor xenograft models, while no harmful effects are observed in normal cells (Fisher et al., 2003; Fisher, 2005; Gupta et al., 2005; Lebedeva et al., 2005a). In a Phase I clinical trial involving adenovirus administration of mda-71IL-24 intratumorally into advanced carcinomas and melanomas, this novel cytokine was found to be safe and demonstrated profound tumor-specific apoptosis induction and significant clinical activity (Fisher et al., 2003; Cunningham et al., 2005; Fisher, 2005; Gupta et al., 2005; Lebedeva et al., 2005a; Tong et al., 2005). Based on these provocative findings clinical trials are ongoing using mda-7/IL-24 for melanoma and other human cancers.

Studies using Ad.mda-7 in melanoma cells establish virally expressed mda-7/IL-24 induces an alteration in the ratio of pro-apoptotic to anti-apoptotic proteins culminating in induction of apoptosis, effects not observed in normal or immortal human melanocytes (Lebedeva et al., 2002). Experiments investigating the mechanism underlying this differential apoptotic effect demonstrated that Ad.mda-7 induced a dose- and time-dependent induction of a family of growth arrest and DNA damage inducible (GADD) genes, GADD153, GADD45α and GADD34, through p38 mitogen activated protein kinase (MAPK) in melanoma, but not in normal immortal melanocytes (Sarkar et al., 2002b). Activation of the GADDs following infection with Ad.mda-7 has also been shown to occur selectively in human malignant glioma and prostate and ovarian carcinomas versus normal primary astrocytes, prostate epithelial cells and mesothelial cells (Su et al., 2003). These findings suggest that induction of these key molecules may be essential for mda-7/IL-24 selective apoptosis-induction in specific cancer cells.

Mda-7/IL-24 is localized on human chromosome 1q32-33 (Blumberg et al., 2001; Huang et al., 2001). The ˜2 kb mda-7/IL-24 mRNA encodes a polypeptide of 206-amino acids. Sequence analysis reveals mda-7/IL-24 is a member of the class-2 cytokine family that includes IL-10, IL-19, IL-20, IL-22, IL-26, and IFN-γ (Pestka et al., 2004). In these contexts, mda-7/IL-24 is expected to adopt an a-helical structure (six α-helices labeled A-F) similar to the crystal structure of IL-10 (Pestka et al. 2004; Walter, 2004; Xu et al., 2004). Consistent with the classification of mda-7/IL-24 as a cytokine, the N-terminal 48-amino acids of the protein form a signal peptide. Expression studies confirm mda-7/IL-24 is secreted as a 1578-amino acid protein that is variably glycosylated at one or more of its three N-linked glycosylation sites (Sauane et al., 2003b).

Like other class-2 cytokines, mda-7/IL-24 binds to cell-surface receptors (IL-20R1/IL-20R2 or IL22R1/IL-20R2 heterodimers) (Dumoutier and Renauld, 2002; Wang et al., 2002), and activates the JAK/STAT signaling pathway (Dumoutier et al.,, 2001; Kotenko et al., 2002; Wang et al., 2002; Pestka et al., 2004). Consistent with its role as a cytokine, exogenously added MDA-7/IL-24 has been shown to induce apoptosis in cancer cells that is dependent on the presence of its cognate cell-surface receptors (Chada et al. 2005; Su et al., 2005b). However, in contrast to the specific action of most cytokines on a few specific cell types, mda-7/IL-24 displays nearly ubiquitous apoptosis-inducing properties in human melanomas, osteosarcomas, fibrosarcomas, mesotheliomas, malignant gliomas and carcinomas of the breast, cervix, colon, liver, lung, nasopharynx, ovary and prostate (Sarkar et al., 2002a; Fisher et al., 2003; Sauane et al., 2003b; Fisher, 2005; Gupta et al., 2005; Lebedeva et al., 2005a). In contrast, no detrimental effect has been observed in a spectrum of normal cells, including skin and lung fibroblasts, melanocytes, mesothelial cells, astrocytes and epithelial cells from the breast, prostate and ovary (Sarkar et al., 2002a; Fisher et al., 2003; Sauane et al., 2003b; Fisher, 2005; Gupta et al., 2005; Lebedeva et al., 2005a).

Studies initially focused on the role of mda-7/IL-24 receptor-mediated JAK/STAT signaling in inducing apoptosis (Sauane et al., 2003a). Using a series of tyrosine kinase (TK) inhibitors, Genistein and AG18, a JAK-selective inhibitor, AG490, and cells defective in specific JAK/STAT signaling pathways studies showed that TK activation was not required for Ad.mda-7-induced apoptosis suggesting that mda-7/IL-24 cancer specific apoptotic activity was JAK/STAT-independent (Sauane et al., 2003a). The ability of this cytokine to kill cancer cells in a JAK/STAT independent manner was also verified using exogenously added recombinant GST-MDA7/IL-24 fusion protein, which induced apoptosis in transformed but not in normal cells as observed for virus-delivered mda-7/IL-24 (Sauane et al., 2004a). Additionally, GST-MDA7/IL-24 protein caused apoptosis in JAK/STAT deficient cell lines and in cells lacking IL-20R1/IL-20-R2 or IL-22R1/IL-20R2 receptors suggesting that mda-7/IL-24-induced cancer specific killing was indeed JAK/STAT-independent and it could occur through mechanisms independent of binding to cognate receptors (Sauane et al., 2004a). Further support for lack of a canonical cytokine mechanism of inducing cancer-specific apoptosis comes from studies using a non-secreted version of mda-7/IL-24 lacking the signal peptide Ad.SP-mda-7 (Sauane et al., 2004b). Virally expressed SP-mda-7 displayed comparable apoptosis inducing activity as full-length Ad.mda-7 (Sauane et al., 2004b). These findings show that mda-7/IL-24-mediated apoptosis can be triggered through an undefined intracellular mode of action as well as via secretion or by a combination of both processes (Sauane et al., 2004b; Su et al., 2005a; Gupta et al., 2005).

Several lines of evidence suggest mda-7/IL-24 intracellular-mediated apoptosis may involve endoplasmic reticulum (ER) signaling. First, localization studies employing Ad.mda-7, Ad.SP-mda-7 and GST-MDA-7/IL-24 indicate that the ER/Golgi compartment is a primary site of localization of MDA-7/IL-24 (Sauane et al., 2004a; Sauane et al., 2004b). Second, Ad.mda-7 induces GADD gene expression that is classically associated with ER stress responses (Sarkar et al., 2002b; Su et al., 2003). Third, Ad.mda-7 infection of H1299 non-small cell lung carcinoma cells leads to to upregulation of IP3R (inositol triphosphate receptor) (Mhashilkar et al., 2003) an ER localized intracellular calcium release channel implicated in apoptosis (Fry, 2001; Rao et al., 2002b).

In addition to cell-based studies, direct interactions have been shown between the class-2 cytokine IFN-γ and the ER resident chaperone BiP/GRP78. Based on amino acid sequence homology among the class-2 cytokines, mda-7/IL-24 also contains one or more putative BiP/GRP78 binding sites that could impact ER signaling responses. Since BiP/GRP78 is involved in binding unfolded polypeptides to promote folding into a 3-D structure, it was possible to identify a deletion mutant of mda-7/IL-24 that would activate ER signaling in a similar manner as wild type mda-7/IL-24. The data show that the ER variant protein BiP/GRP78 acts as an intracellular target for MDA-7/IL-24, documenting the importance of binding with subsequent activation of the down stream targets p38 MAPK and GADD in mediating apoptosis selectively in cancer cells. A truncated version of MDA-7/IL-24 was also identified, M4 consisting of amino acids 104 to 206 of the full-length protein, that retains BiP/GRP78 binding, localizes in the ER and induces biochemical changes promoting growth suppression and apoptosis uniquely in tumor cells, both in vitro and in vivo. The present studies also show a non-canonical intracellular mode of apoptosis-induction by the IL-10 family member mda-7/IL-24 and suggest a small molecule mimetic of mda-7/IL-24 activity may be developed that selectively induces apoptosis in cancer cells.

Results

Mapping Functional Regions of mda-7/IL-24 That Mediate Cancer-specific Growth Suppression

To test the hypothesis that mda-7/EL-24 induces apoptosis through an intracellular receptor-independent mechanism, a series of mda-7/IL-24 deletion mutants (M1-M6) were constructed. The mutants were guided by secondary structure predictions of MDA-7/IL-24 defined by amino acid sequence and structural homology with IL-10 (Walter and Nagabhushan, 1995). In the first mutant (M1), the signal peptide that directs secretion of mda-7/IL-24 is deleted (Sauane et al., 2004b). In M2, the signal peptide and residues prior to α-helix A are deleted. Mutants M3-M6 correspond to peptides that contain putative MDA-7/IL-24 α-helices B, C, D, E and F (M3), C, D, E, and F (M4), D, E, and F (M5), and E and F (M6). This strategy was adopted to define fragments of MDA-7/IL-24 that might be biologically active even if they cannot adopt a completely folded three-dimensional structure or be secreted into the culture media to bind cell-surface receptors.

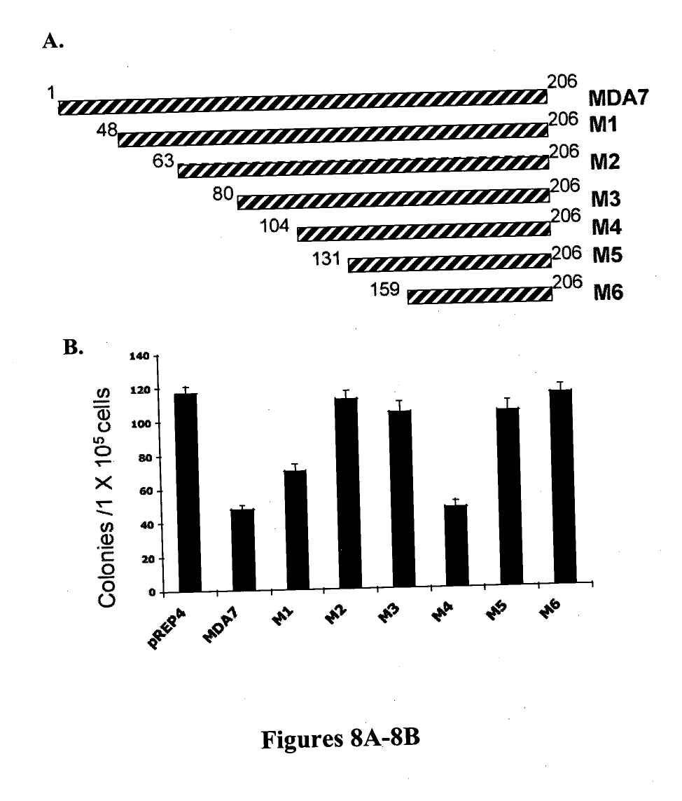

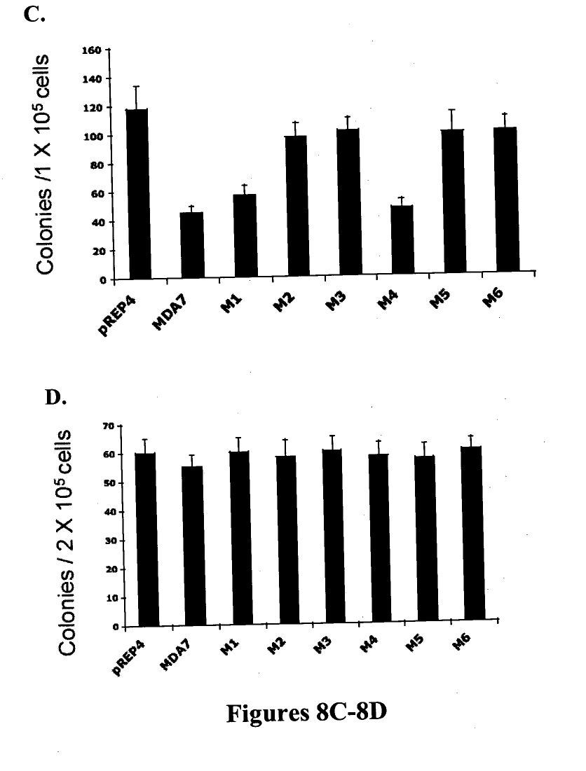

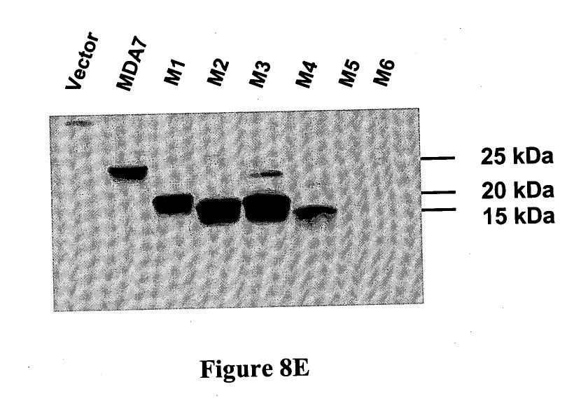

Mutants M1-M6 were transiently expressed in cancer (HeLa and DU-145) and normal (P69) cell lines and their ability to suppress cell growth was evaluated (FIGS. 8B, 8C and 8D). M1, which lacks the signal peptide amino acids 1-47, had significant growth suppressive properties in HeLa and DU-145 cells (FIGS. 8B and 8C), without altering growth in SV40-immortalized normal human prostate epithelial (P69) cells (FIG. 8D). Deletion of residues from 1-62 and 1-79 of the full-length mda-7/IL-24 gene in constructs M2 and M3, respectively, resulted in molecules that were devoid of growth suppressive activity (FIG. 8). Deletion of residues 1-103 (M4, corresponding to α-helices C, D, E, and F) retained the functional activities of the full-length MDA-7/IL-24 gene product, inducing cancer-specific growth suppression in HeLa and DU-145 cells (FIGS. 8B and 8C). Transfection of the M4 construct into additional cancer cell lines, including LNCaP (prostate carcinoma) and T47D (breast carcinoma) decreased colony formation, whereas no colony inhibition was observed in P69 (FIG. 8D) or FM516-SV (an SV40 T-antigen immortalized normal human melanocyte cell line). Further deletion of residues 1-130 (M5) or 1-159 (M6) rendered the molecule inactive in cancer-specific cell growth suppression activity.

To characterize the expression levels of the mutants, M1-M6 were subcloned into a pCMV3×Flag vector. The Flag M1-M6 were expressed in HeLa, DU-145, and P69 cells and quantified by Western blotting using an anti-Flag antibody. Expression of deletion mutants from the flag-tagged constructs revealed that functional protein was synthesized for full-length MDA-7/IL-24, M1, M2, M3 and M4 (FIG. 8E). However, no flag-tagged proteins were detected for the M5 or M6 constructs (FIG. 8E).

Ectopic Expression of M4 by Adenovirus Induces Cancer Cell-specific Apoptosis

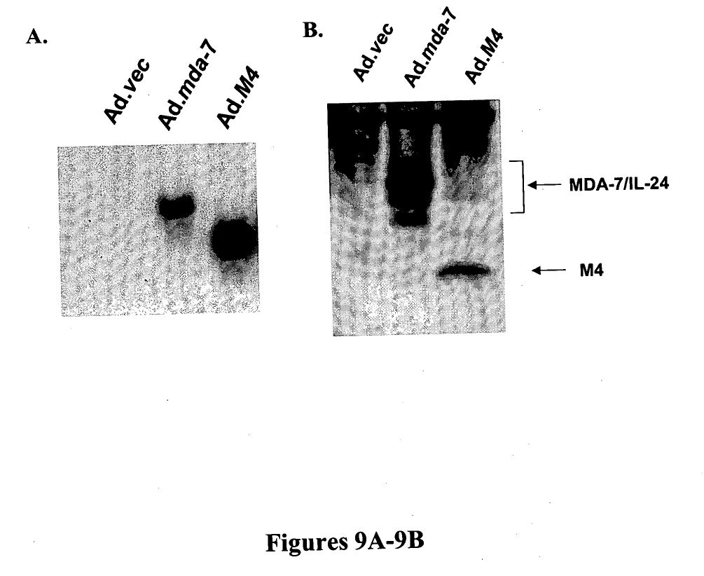

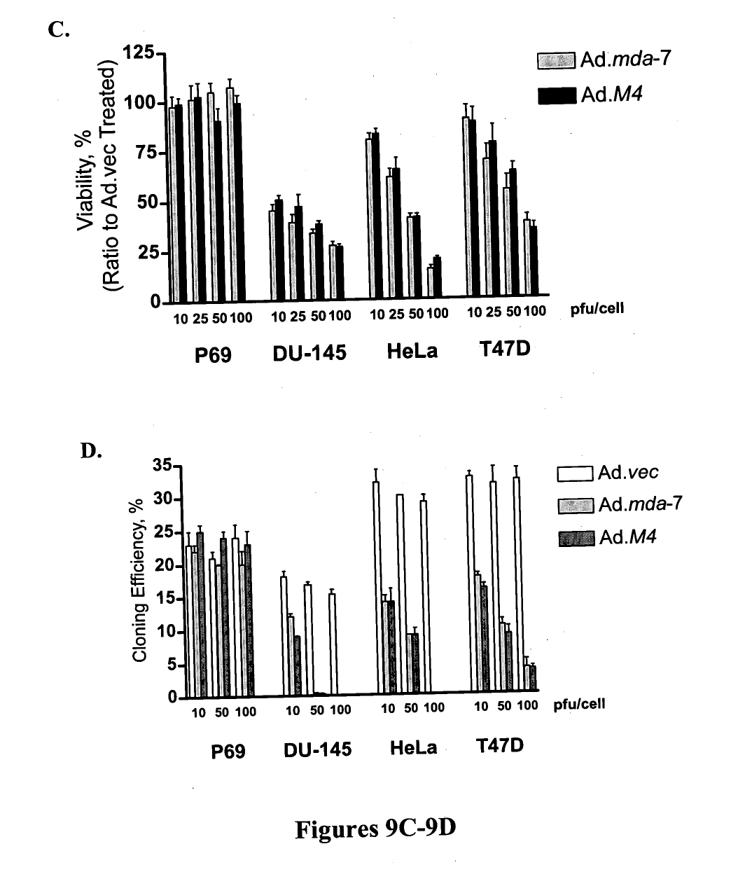

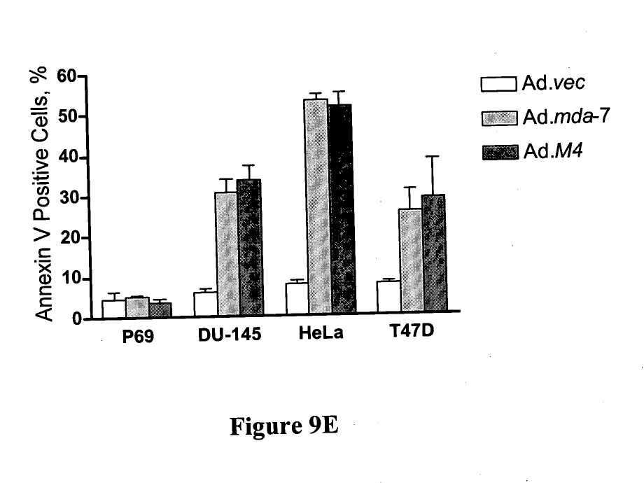

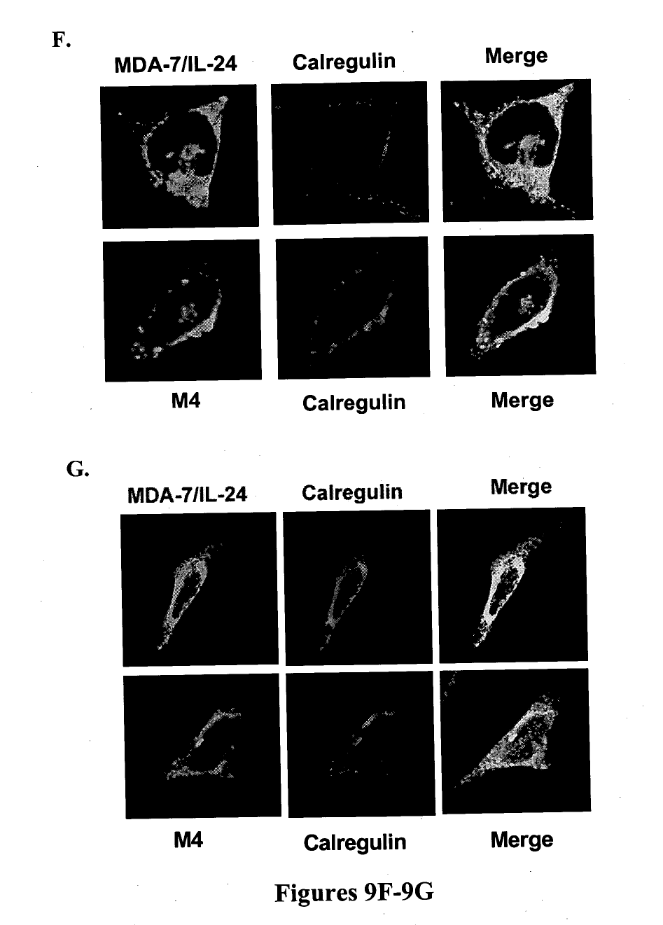

Ectopic expression by means of an adenovirus provides efficient delivery of gene products in proliferating and non-proliferating cells permitting evaluation of biological function of wild type and mutant suppressor genes. To begin to define how M4, which contains literally one-half of the novel cytokine MDA-7/IL-24, elicits similar cancer-specific growth suppressing properties as the full-length molecule, a replication incompetent type 5 adenovirus expressing M4, Ad.M4, was constructed. HeLa cells were infected with 50 pfu/cell of Ad.vec (Ad lacking a gene insert), Ad.mda-7 (Ad containing the full-length mda-7/IL-24 without UTRs) or Ad.M4 (Ad containing the M4 mutant), 24 hr later RNA was isolated and Northern blotting was performed (FIG. 9A). Additionally, cells were lysed and levels of MDA-7/IL-24 and M4 proteins were determined by Western blotting (FIG. 9B). Infection of HeLa cells with Ad.M4 produced a single MDA-7/IL-24 protein of ˜15-kDa, whereas Ad.mda-7 generated multiple bands, because of glycosylation, ranging in size from ˜20- to ˜25-kDa (FIG. 9B). Similar results were obtained when normal primary human fetal astrocytes (PHFA), FM516-SV or P69 cells were infected with Ad.mda-7 or Ad.M4.

The impact of Ad.M4 and Ad.mda-7 virus infection on the survival of cancer and normal cells was evaluated. HeLa, DU-145 and P69 cells were infected with 100 pfu/cell of Ad.vec or 10, 25, 50 or 100 pfu/cell of Ad.M4 or Ad.mda-7. Cell viability was monitored by MTT assays performed at days 1, 3 and 5. These experiments confirmed a dose-dependent decrease in cell viability in DU-145 and HeLa cells following infection with Ad.M4 or Ad.mda-7 (FIG. 9C). In contrast, no discernible effect was evident on viability of P69 cells even after infection with 100 pfu/cell of Ad.M4 or Ad.mda-7 (FIG. 9C). Definitive decreases in cancer cell viability were evident with as little as 10 pfu/cell of Ad.M4 or Ad.mda-7 in DU-145 and HeLa cells (FIG. 9C). Inhibition of long-term viability was documented using clonal survival assays (FIG. 9D). In these experiments, Ad.M4 and Ad.mda-7 resulted in a profound decrease in survival of DU-145 and HeLa cells, even when cells were infected with 10 pfu/cell of virus. In contrast, as had been observed in the MTT assays, no significant effect was apparent on colony formation versus Ad.vec infected P69 cells, even when infected with 100 pfu/cell of Ad.M4 or Ad.mda-7. These studies confirm similar restricted anti-proliferative and anti-survival effects of Ad.M4 versus Ad.mda-7 in cancer cells, with no apparent toxic effects in normal cells.

Annexin V staining, which monitors early apoptotic changes in cells, was determined by FACS analysis in P69, DU-145, HeLa and T47D (breast carcinoma) cells 24 hr after infection with 100 pfu/cell of Ad.vec, Ad.M4 or Ad.mda-7 (FIG. 9E). Infection of P69 cells with Ad. vec, Ad.M4 or Ad.mda-7 resulted in ˜5-8 % Annexin V positive stained cells, while ˜35-40% of DU145, ˜50-55% of HeLa and ˜25-30% of T47D cells stained Annexin V positive after infection with Ad.M4 or Ad.mda-7 (FIG. 9E). These results show that both M4 and MDA-7/IL-24 display similar apoptotic-inducing properties in cancer cells, without prompting apoptosis in normal cells. A lack of apoptosis-inducing properties was also apparent in normal PHFA and FM516-SV cells. These results show that the M4 mutant, which consists of four of the six putative α-helices of MDA-7/IL-24 (α-helices C, D, E and F), retains the same cancer-specific growth-suppressive and apoptosis-inducing properties as the full-length molecule. Furthermore, M4 does not contain a signal sequence and is not secreted from cells. This data provides additional support for a novel intracellular mode of cancer cell-specific killing by mda-7/IL-24.

M4, Like mda-7/IL-24, Localizes in the Endoplasmic Reticulum

Mda-7/IL-24 can induce cancer cell-specific killing that is not dependent on interactions with the canonical Il-20/IL-22 receptor chains or the JAK/STAT signaling pathway (Sauane et al., 2003a; Su et al., 2005a). This provokes the obvious question of what intracellular target might mediate the selective intracellular killing of cancer cells by mda-7/IL-24. Previous studies revealed MDA-7/IL-24 localizes in the ER in both normal and cancer cells prompting us to determine if M4 also localizes to the ER.

To address this question, the subcellular localization of M4 and MDA-7/IL-24 was analyzed in DU-145 and P69 cells after infection with Ad.M4 or Ad.mda-7 (FIGS. 9F and 9G). Immunofluorescence detection was standardized at different time points to avoid ambiguous changes in localization that might occur as a result of loss of internal membrane integrity due to apoptotic events induced by M4 or MDA-7/IL-24 in DU-145 cancer cells. Like full-length MDA-7/IL-24 protein, M4 was localized in the ER compartment in both cancer and normal cells (FIGS. 9F and 9G).

Hydrophobic Residues in the C and F Helices of the M4 and MDA-7/IL-24 Proteins are Required for Biological Activity

Studies by Vandenbroeck et al. (2002) identified a conserved DnaK/BiP/GRP78 binding site in all IL-10 family members, including mda-7/IL-24 that may be necessary to assist in the folding of these molecules (Vandenbroeck et al., 2002). The conserved DnaK/BiP/GRP78 binding site is located on helix C and consists of the eight-residue sequence TLLEFYLK in mda-7/IL-24. In the three-dimensional structure of IL-10 and IFN-γ, the helix C DnaK/BiP/GRP78 binding site is positioned next to a second highly conserved amino acid sequence (KALGEVD in mda-7/IL-24) located in helix F. In contrast to the conserved segment in helix C, the conserved sequence in helix F has not been shown to interact with DnaK/BiP/GRP78. Because MDA-7/IL-24 and M4 both localize to the ER upon expression in cells, a potential role of these conserved residue segments, located in helices C and F, was investigated in mediating killing by M4 and MDA-7/IL-24.

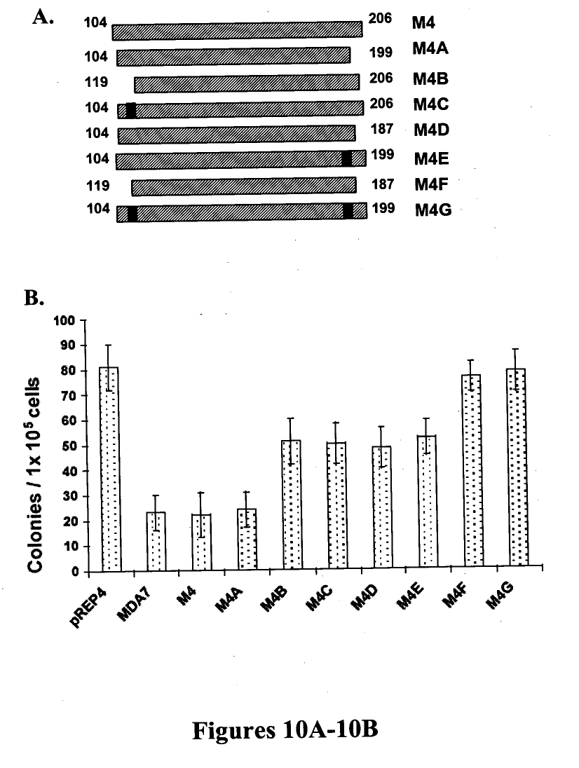

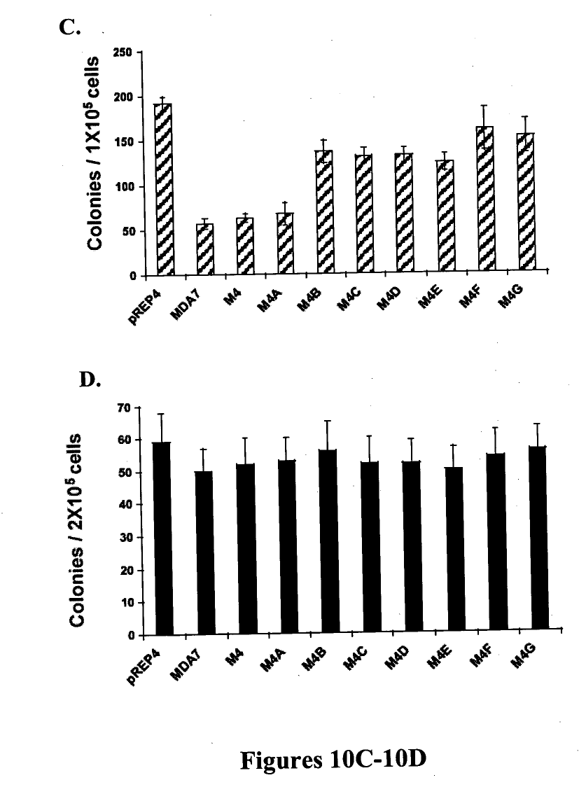

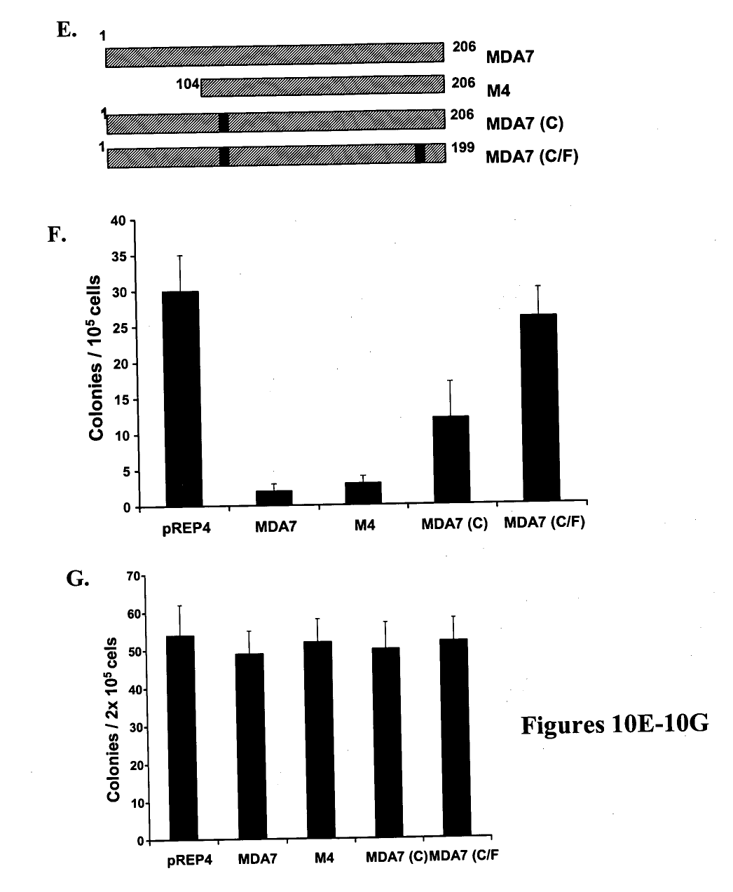

To explore the role of conserved residues in helices C (TLLEFYLK) and F (KALGEVD) of MDA-7/IL-24 in inducing cancer cell-specific killing, a second set of mutants was made (M4A-M4G). The last 7 residues of MDA-7/IL-24 were deleted in M4A, resulting in the mutant containing residues 104-199. M4B (residues 119-206) corresponds to a deletion of helix C, which contains the DnaK/BiP/GRP78 binding site. M4C is the same length as M4 (104-206), but helix C residues TLLEFYLK, were mutated to AGDATAGA. In M4D, the entire F helix was deleted and the construct retained residues 104 to 187 of MDA-7/IL-24. In M4E, conserved residues in helix F, (KALGEVD), were mutated to GAHGAVA. M4F (residues 119-187) is a double deletion mutant where both MDA-7/IL-24 helices C and F were removed. Finally, M4G is a double mutation construct where both the conserved residues in helices C and F were mutated as previously described for mutants M4C and M4E (FIG. 10A).

The various constructs were evaluated for functional activity using colony (clonal) formation assays in HeLa and DU-145 cancer cells and in normal P69 cells. These experiments demonstrated that mda-7/IL-24, M4, and M4A reduced hygromycin resistant colony formation relative to control pREP4 transfected cells to an equivalent degree in both HeLa and DU-145 cells, without significantly altering colony formation in normal P69 cells (FIGS. 10B, 10C and 10D). Mutations or deletions of either the C or F helices, M4B, M4C, M4D and M4E, modestly reduced colony formation in HeLa and DU-145 cells as compared with transfection with mda-7/IL-24, M4 or M4A. The M4F and M4G mutants, which contain mutations or deletions in both the C and F helices, were devoid (HeLa) or displayed minimal (DU-145) colony inhibitory activity (FIGS. 10B and 10C). None of these additional mutants affected colony formation in P69 cells (FIG. 10D). These data show a role for the C and F helices of the MDA-7/IL-24 protein in mediating cancer-specific activity of the M4 deletion mutant of mda-7/IL-24. When either site was mutated or deleted there was a disruption of the activity and functionality was essentially extinguished when both sites were deleted or mutated.

To examine further the role of helix C and F residues in mediating killing by MDA-7/IL-24, mutations were generated in the conserved regions of helices C and F in full-length MDA-7/IL-24. In MDA7 (C), helix C of MDA-7/IL-24 was mutated from TLLEFYLK to TLAGSRLG, and in MDA7 (C/F) both the C and F helices of MDA-7/IL-24 were mutated. In mutant MDA-7 (C/F), α-helix C residues TLLEFYLK were mutated to residues TLAGSRLG and α-helix F residues KALGEVD were mutated to residues GAHGAVA. (FIG. 10E). The mutation introduced in MDA-7 (C) is different than other helix C mutations made in M4 due to difficulties in generating the construct, while mutations in the conserved region of helix F were identical to those used in the M4 constructs.

The effect of these mutations on colony formation in cancer and normal cell lines using colony formation assays was evaluated (FIGS. 10F and 10G). Mutations in helix C disrupted the functional activity of MDA-7/IL-24 and mutations in both the C and F (C/F) helices abrogated the cancer-specific inhibitory activity of MDA-7/IL-24, resulting in a similar number of colonies as observed in pREP4 vector-transfected cultures (FIG. 10F). However, these colonies were morphologically smaller than colonies formed in vector control transfected cells, suggesting retention of some growth modulating activity. No effect was observed in P69 cells with either of these MDA-7/IL-24 mutants (FIG. 10G). These studies confirm that both the C and F helices of the MDA-7/IL-24 protein are crucial for maintaining optimum mda-7/IL-24 cancer-specific growth suppressive activity.

ER Variant BiP/GRP78 Interacts With MDA-7/IL-24 and M4

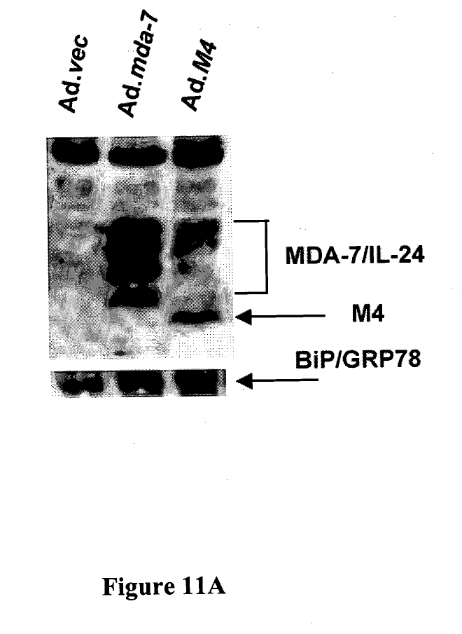

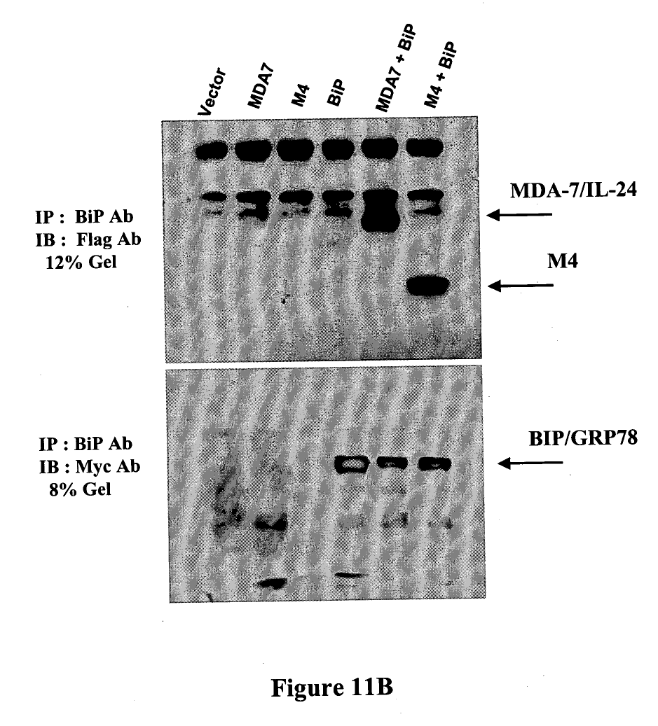

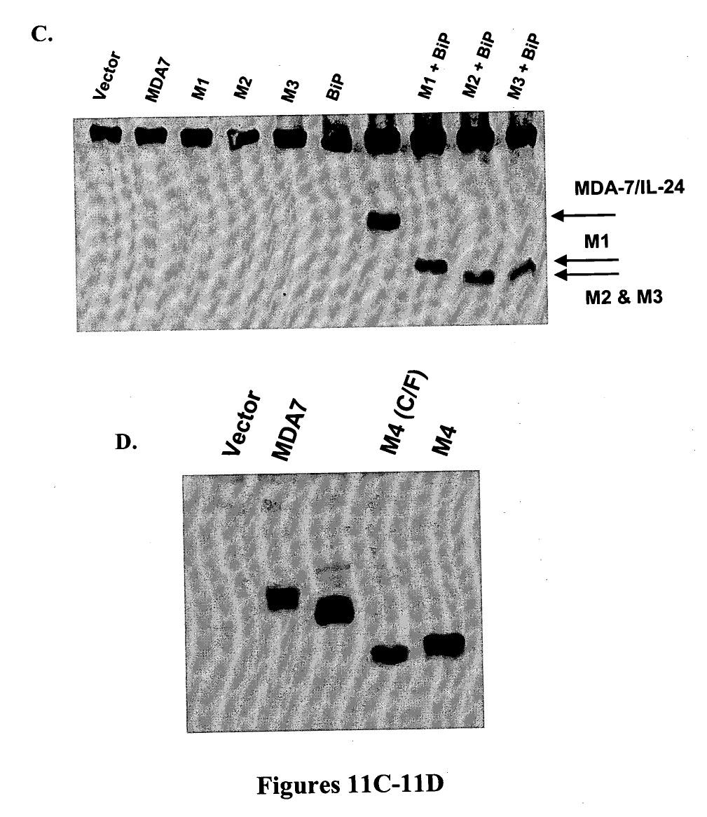

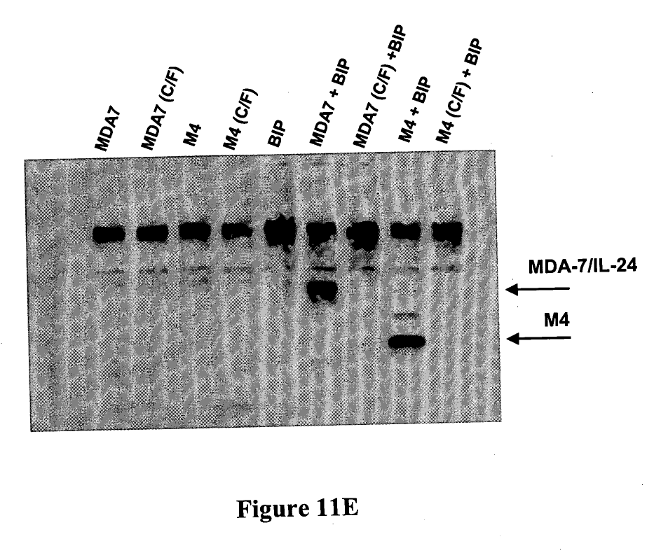

These data show a potential involvement of conserved residues in α-helices C and F in mediating the cancer-specific inhibitory activity of both M4 and full-length MDA-7/IL-24. Because the conserved region of helix C in IFN-γ has been shown to interact with DnaK and BiP/GRP78, experiments were performed to investigate whether BiP/GRP78 and MDA-7/IL-24 bind to one another. Ectopic expression of MDA-7/IL-24 and M4 protein by adenovirus transduction followed by immunoprecipitation (IP) using BiP/GRP78 antibodies confirmed a physical interaction between these molecules (FIG. 11A).

To further explore the interaction between BiP/GRP78 and MDA-7/IL-24 or M4, MDA-7/IL-24 or M4 was transiently expressed in HeLa cells to characterize BiP/GRP78 interactions. However, as seen in FIG. 11A, an interaction between BiP/GRP78 and MDA-7/IL-24 or M4 was only evident when cells were infected with Ad.mda-7 or Ad.M4. To overcome this problem, MDA-7/IL-24 and BiP/GRP78 were simultaneously expressed with different affinity tags, i.e., Flag or Myc, and Co-IP analyses was repeated (FIG. 11B). Flag-tagged MDA-7/IL-24 or M4 were transiently transfected with myc-tagged BiP/GRP78 into HeLa cells and IP was performed using 9E10 Myc monoclonal antibodies. The protein samples were electroblotted and developed with the Flag antibody M2. As can be seen in FIG. 11B, MDA-7/IL-24 and M4 Co-IP with BiP/GRP78 demonstrating a physical interaction between these two molecules. Co-IP of MDA-7/IL-24 and M4 with BiP/GRP78 was also observed when polyclonal BiP/GP78 antibody was used for IP. Similarly, cotransfection of HeLa cells with Flag-tagged M1, M2 or M3 with BiP/GRP78 resulted in Co-IP with 9E10myc antibodies confirming interaction of these MDA-7/IL-24 mutants with BiP/GRP78 (FIG. 11C). Experiments were also performed in a reverse direction. For example, IP was performed using Flag antibodies and the membrane was probed with the Myc antibody 9E10. This experiment also confirmed IP/GRP78 interaction with MDA-7/IL-24 and M4, as well as M1, M2 and M3, only when both molecules were simultaneously transfected into HeLa cells.

To investigate further the putative roles of the C and F helices of MDA-7/IL-24 and M4 in mediating interaction with BiP/GRP78, flag tagged mutants of MDA-7/IL-24 and M4 were constructed at the C as well as the F helices. FIG. 11D shows expression of the flag-tagged wild type MDA-7/IL-24 and M4 as well as the C plus F (C/F) mutants of MDA-7/IL-24 and M4. These mutants were used to examine whether disruption of these regions altered interaction with BiP/GRP78 in HeLa (FIG. 11E) and P69 cells. BiP/GRP78 was immunoprecipitated using BiP/GRP78 antibodies and the membrane was probed with Flag antibodies (FIG. 11E). The C/F helix mutants of MDA-7/1IL-24 and M4 lost their ability to bind to BiP/GRP78 (FIG. 11E). Similar results were obtained using normal P69 cells, indicating that BiP/GRP78 binding is dependent on the integrity of the conserved residues located in helices C and F. These studies confirm that BiP/GRP78 interacts with MDA-7/IL-24 and M4 through the conserved residues in helices C and/or F and mutation of these residues prevents binding and abrogates the cancer-specific apoptosis inducing properties of full-length MDA-7/IL-24 as well as M4. These studies confirm MDA-7/IL-24-BiP/GRP78 interactions occur in both normal and cancer cells and that this physical interaction by itself, although necessary for apoptosis-induction in cancer cells, does not mediate growth suppression or apoptosis induction by this novel cytokine in normal cells.

M4 Induces Activation of p38 MAPK and GADD Gene Family Expression

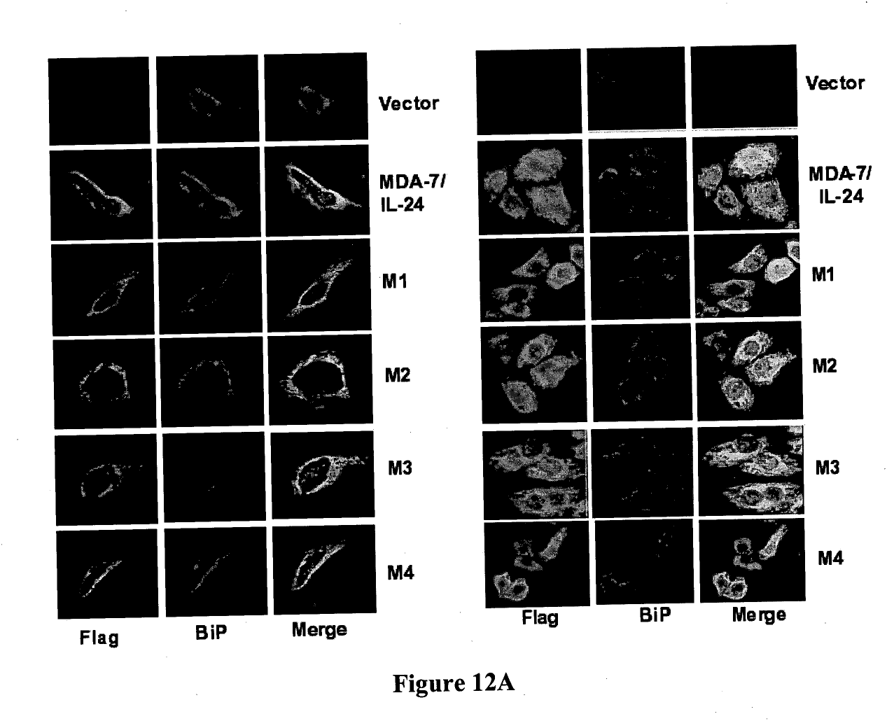

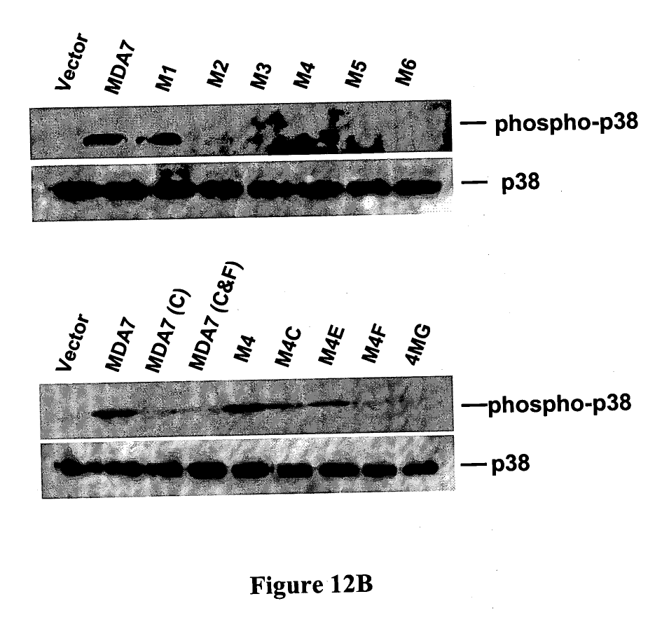

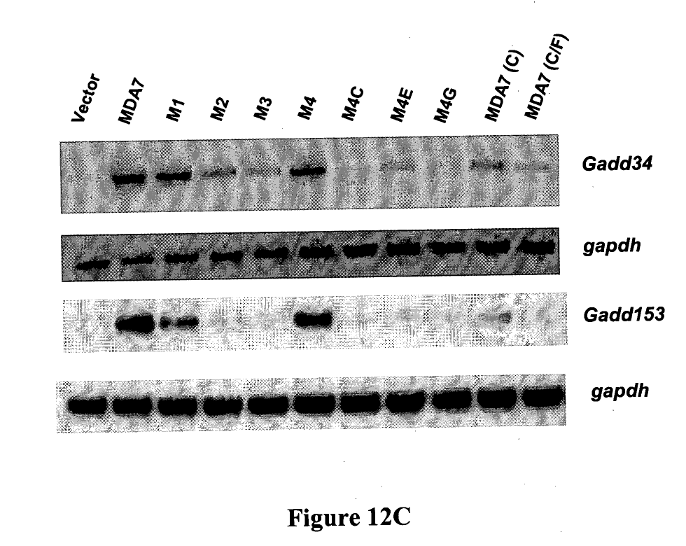

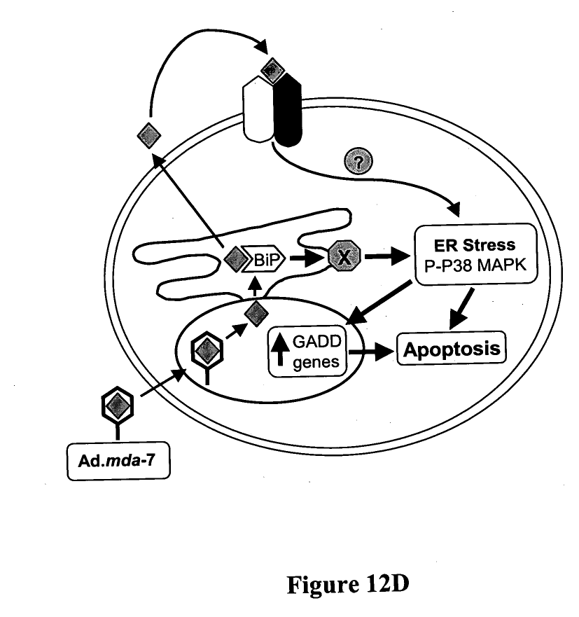

Previous observations have shown MDA-7/IL-24 activates p38 MAPK in a number of target cancer cells, but not normal cells, thereby resulting in induction of the pro-apoptotic GADD family of genes causing apoptosis selectively in cancer cells (Sarkar et al., 2002b; Su et al., 2003). Blocking p38 MAPK activation in melanoma cells using pharmacological inhibitors or through a dominant-negative strategy or antisense blocking of GADD gene expression inhibits or reduces, respectively, apoptosis induction by Ad.mda-7 (Sarkar et al., 2002b). These findings argue that this signaling pathway is relevant for apoptosis induction by mda-7/IL-24 in specific cancer cells. Based on these considerations, various MDA-7/IL-24 and M4 mutants, including M1 (lacking the signal peptide, amino acids 1 to 48), M2, M3, M4 and various point or deletion mutations in M4, were tested to determine if they retain the ability to induce phoshorylation of p38 MAPK and induce GADD gene expression. As shown in FIG. 12B, full length MDA-7/IL-24, M1 and M4 proteins retain the ability to maximally promote p38 MAPK phosphorylation. In contrast, M2, M3, M4 and M5 do not induce p38 MAPK phosphorylation. Analysis of the downstream targets of p38 indicated maximum induction of both GADD34 and GADD153 mRNA by MDA-7/IL-24, M1 and M4 (FIG. 12C). Similarly, reduced phosphorylation of p38 MAPK and induction of GADD34 and GADD153 mRNA was apparent in variants encoding helix C or helix C plus F (C/F) mutants of full-length MDA-7/IL-24, MDA7 (C) and MDA7 (C/F), and in the M4 mutants M4C, M4E, M4F and MFG (FIG. 12C). Although the mechanism by which MDA-7/IL-24 and M4 induce p38 MAPK phosphorylation remains to be determined, the present study identifies a relevant downstream target gene family that is activated after BIP/GRP78 binding and which is critical for MDA-7/IL-24 and M4 to induce apoptosis selectively in cancer cells (FIG. 12D).

M4 Retains Antitumor Properties in Vivo in a Human Tumor Nude Mouse Xenograft Model

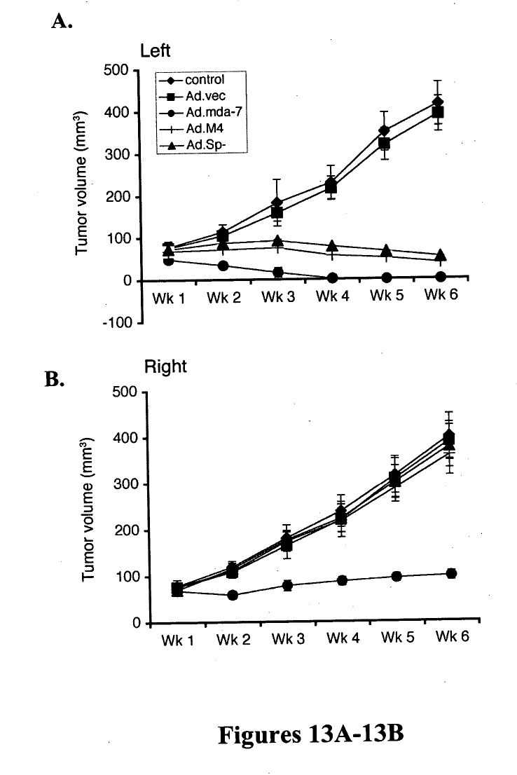

Ad.mda-7, which expresses the full-length mda-7/IL-24 gene, has potent antitumor activity in nude mice containing human tumor xenografts {Su et al., 1998; Madireddi et al., 2000; Sarkar et al., 2005). Based on this consideration, studies were designed to determine if M4, administered by adenovirus, would display antitumor activity and how it would compare with Ad.mda-7. Since M4 lacks a signal peptide, plus an additional 54 amino acids of the full length MDA-7/IL-24, the effect of an adenovirus expressing an M1 gene construct, Ad.Sp-mda-7 (Sauane et al., 2004b) was also tested. For the tumor studies, a scheme known in the art (Sarkar et al., 2005) was utilized in which tumors were established on both sides of an animal and the therapeutic agent was applied to one side of the animal and its effect on the injected and non-injected tumor sites were determined over time. This approach provides insight into “antitumor bystander” activity (Su et al., 2001; Su et al., 2005b; Chada et al., 2005), which is an inherent property of mda-7/IL-24 that significantly increases its therapeutic utility {Fisher et al., 2003; Lebedeva et al., 2005b; Tong et al., 2005; Fisher, 2005). Intratumoral injection of Ad.M4 and Ad.Sp-mda-7 in established T47D human breast cancer xenografts in nude mice significantly inhibited tumor growth on the left side (injected site) when compared to that of control (untreated) or Ad.vec (control empty adenovirus) injected animals (FIGS. 13A and 13B). However, Ad.M4 and Ad.Sp-mda-7 exerted no discernible effect on the tumors on the uninjected right side. However, injection of Ad.mda-7 completely eradicated tumors on the left side and markedly inhibited the growth of the tumors on the right side. These findings indicate that although Ad.M4 and Ad.Sp-mda-7 significantly inhibited tumor growth, because of the lack of secretory ability, they did not show any “antitumor bystander” activity. In contrast, Ad.mda-7 eradicated primary (left-sided) and significantly inhibited distant (right-sided) tumors indicating that it has potent “antitumor bystander” activity.

Discussion

To define the mechanism by which MDA-7/IL-24, an IL-family cytokine, selectively induces apoptosis in cancer cells without interactions with its cell-surface receptors (EL-20R1/IL-20R2, IL-22R1/IL-20R2) and without JAK/STAT activation, a series of MDA7/IL-24 deletion mutants were constructed and evaluated for growth-suppressing and apoptosis-inducing activity in cancer and normal cells. This analysis revealed the MDA7/IL-24 deletion mutant containing amino acids 104 to 206 (M4), exhibits apoptosis-inducing activities indistinguishable from the full-length protein.

The MDA-7/IL-24 deletion mutant M4 lacks the signal sequence and two (α-helices, A and B) of the six putative α-helices of the wild-type protein. Despite the deletion of 50% of the MDA-7/IL-24 amino acid sequence, the M4 mutant selectively induces apoptosis in cancer cells by activating p38 MAPK and promoting GADD34 and GADD153 gene expression. MDA-7/IL-24 mutants, M1 (lacking the signal sequence) and M4, were also evaluated in nude mice containing human breast tumor xenografts established on both sides of the animals (Sarkar et al., 2005). In this model, Ad.mda-7 injected in the left flank of the animals efficiently reduced the size of tumors in the left as well as the right flanks of the mice. In contrast, Ad.M1 and Ad.M4 reduced the size of tumors at the site of virus injection (left flank), but had no effect on the tumor located on the right flank of the animal. Because Ad.M1 and Ad.M4 do not contain signal sequences, the data support an intracellular mechanism of MDA-7/IL-24 anti-tumor action. Furthermore, since at least 50% of the putative MDA-7/IL-24 receptor binding sites has been deleted in the M4 mutant, it appears that MDA-7/IL-24 receptor interactions are not essential for its apoptosis-inducing activity. However, as shown in the xeongraft tumor model, secreted MDA-7/IL-24 can induce cancer cell apoptosis in a paracrine manner (e.g., ‘bystander activity’) at distantly located tumors, presumably by receptor-mediated mechanisms (Chada et al., 2005; Su et al., 2005b). Further studies are required to determine if addition of a secretory signal to M4 will permit this truncated MDA-7/IL-24 protein to induce ‘bystander activity’.

A requirement for MDA-7/IL-24-, M1-, and M4-mediated cancer cell apoptosis is an interaction with the ER chaperone BiP/GRP78. Disrupting MDA-7/IL-24: BiP/GRP78 or M4: BiP/GRP78 interactions by mutating the conserved BiP/GRP78 binding site in helix C prevented cancer cell apoptosis and the activation of p38 MAPK and the GADD genes. These results suggest that MDA-7/IL-24 binding to the chaperone BiP/GRP78 in a cancer cell-specific context may induce ER stress signals and ultimately apoptosis by activating the GADD genes through p38 MAPK (FIG. 12D).

MDA-7/IL-24 mutants M2 and M3, which do not induce apoptosis, have intact BiP/GRP78 binding sites in helices C and F. Thus, M2 and M3 mutants bind BiP/GRP78 and localize to the ER, but these mutants did not induce similar p38 MAP K phosphorylation or GADD gene expression. These results suggest BiP/GRP78 binding is required, but not sufficient, for MDA-7/IL-24-mediated cancer cell apoptosis. The results are consistent with BiP/GRP78's primary role of assisting in the proper folding of a variety of secreted proteins, including other members of the IL-10 family which contain conserved BiP/GRP78 binding sites in their sequences (Vanderbrock et al., 2002). Inactive M2 and M3 mutants both contain residues 104-206 of MDA-7/IL-24 (M4), which is able to selectively induce apoptosis in cancer cells. Considering that these inactive constructs interact with BiP/GRP78, apparently, in addition to stabilization by chaperones, a higher level of regulation may be operational to control the activity of MDA-7/IL-24. This regulation might be mediated by the interaction of MDA-7/IL-24 with as yet unidentified protein(s) (Protein X) in addition to BiP/GRP78 to which M1 and M4, but not M3 or M3, interact thereby activating downstream signaling cascades, such as p38 MAPK phosphorylation and subsequent GADD gene induction (FIG. 12D).

At least some insight into the connection between MDA-7/IL-24: BiP/GRP78 interactions and cancer cell apoptosis can be obtained from recent studies on the activation mechanisms of the ER stress response (Rao et al., 2002a; Rao et al., 2004; Rao et al., 2002b). In particular, the activation of the membrane associated transcription factor ATF6, which induces several ER stress response genes, is controlled by a competition between the luminal domain of ATF6 and unfolded proteins in the ER for BiP/GRP-78 (Shen et al., 2002; Shen and Prywes, 2004; Shen et al., 2005). Under normal conditions, ATF6 is kept in a sequestered inactive form by interactions with BiP/GRP-78. However, dissociation of BiP/GRP78 from ATF6 activates the transcription factor, which results in the induction of several ER stress response genes (Yoshida et al., 1998). Thus, in cancer cells, the expression of MDA-7/IL-24, M1, and M4 may compete for BiP/GRP78 leading to ATF6 activation, and possibly other signaling molecules that regulate cancer cell apoptosis. In contrast, the additional MDA-7/IL-24 residues found in the inactive M2 and M3 mutants, may shield or prevent high-affinity interactions with BiP/GRP78 and possibly other ER proteins that regulate apoptosis (FIG. 12D). These studies also show the ER is extremely sensitive to the type of protein/peptide required to induce BiP/GRP78-mediated cancer cell apoptosis.

Based on its multifunctional therapeutic properties (Fisher, 2005; Fisher et al., 2003; Gupta et al., 2005; Lebedeva et al., 2005a), mda-7/IL-24 is being hailed as a potential ‘magic bullet’ for cancer gene therapy (Fisher, 2005) and M4 may provide a means of further enhancing its applications as an anti-tumor agent. Because of its small size, the delivery of M4 is predicted to be more efficient thereby augmenting in vivo activity. A number of cytokines are currently being evaluated for cancer gene therapy, including IL-2, IL-4 and IL-12, which exert their anti-tumor effects predominantly by modulating the immune system. Among them, mda-7/IL-24 belongs to a highly select group, perhaps only rivaled by interferon, that can directly induce apoptosis, promote profound ‘bystander activity’, inhibit angiogenesis, augment anti-tumor immune responses and promote radiosensitization (Fisher et al., 2003; Fisher, 2005; Gupta et al., 2005; Lebedeva et al., 2005a). Moreover, these results describe a novel mechanism of action and properties of MDA-7/IL-24 providing an opportunity to develop strategies for augmenting its potential as a therapeutic agent (Fisher, 2005).

In summary, MDA-7/IL-24 is an α-helical cytokine that has tremendous potential as a gene therapy for cancer. These studies identified a peptide of MDA-7/EL-24 (M4, residues 104-206) that mimics the biological properties of the full-length protein. The Experimental evidences confirm that interactions with the ER chaperone BiP/GRP78 are critical for the ability of MDA-7/IL-24 or M4 to induce cancer cell apoptosis. This data provides and explanation for how virally expressed MDA-7/IL-24 induces apoptosis without the need for cell-surface receptor interactions or the JAK/STAT signaling pathway (Sauane et al., 2003a). It also provides a possible explanation for why MDA-7/IL-24 is able to kill diverse types of cancer cells. Finally, the effectiveness of MDA-7/IL-24 in selectively inducing apoptosis in cancer cells, but not normal cells, is consistent with cancer cells already being under significant metabolic stress. Thus, MDA7/IL-24 peptides may lead to new therapeutics that selectively target and kill cancer cells based on their increased level of stress compared to normal cells.

Materials and Methods

Cell Culture and Transfection Assays

HeLa (human cervical carcinoma), DU-145 (human prostate carcinoma), T47D (human breast carcinoma), and FM516-SV (SV40 T Ag-immortalized normal human melanocyte) cell lines were grown in Dulbecco's modified Eagle's medium (DMEM) supplemented with 10% fetal calf serum at 37° C. in a 5% CO₂ incubator. SV40 T Ag-immortalized normal human prostate epithelial cells P69 were grown in serum free media supplemented with EGF (Bae et al., 1994). Primary human fetal astrocytes (PHFA) were grown in DMEM with 10% fetal calf serum. The various cell types were transfected using Lipofectamine 2000 (Invitrogen) or Superfect (Qiagen) according to the manufacturer's instruction and were incubated for 24 to 48 h before further experimental manipulation was performed as outlined in specific figure legends.

Construction of MDA-7/IL-24 Mutants

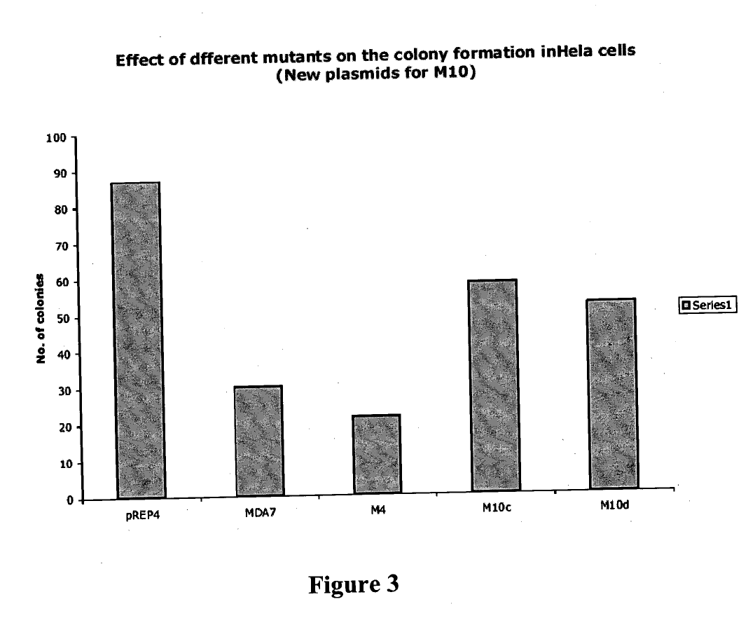

Serial N-terminal deletion mutants of MDA-7/IL-24 (M1 to M6) were generated by PCR using a common antisense primer and corresponding sense primers (Table 1; Supplemental Data). M1 (a.a. 48-206) was devoid of the signal peptide. In M2 (a.a. 63-206) the α-helical domain A was disrupted in the middle. In M3 (a.a. 80-206), the α-helical domain B was disrupted in the middle. M4 (a.a. 104-206) contained the C, D, E and F□-helical domains. M5 (a.a. 131-206) contained only the D, E and F α-helical domains, while M6 (a.a. 159-206) contained the E and F α-helical domains. A Kozak sequence including the start codon (GCCACCATG) was added in front of the mutants for better expression. Mutants were cloned into the HindIII and BamHI sites of the vector pREP4 (Invitrogen), which contains a hygromycin resistance gene selection marker. Similar deletion mutants were also made in the HindIII and BamHI sites of the vector pCMV3×Flag (Sigma) containing three flag tags at the N-terminus. Additional mutations were made in deletion mutant M4 (M4A to M4G) by PCR using the primers as described in Table 1 (FIG. 3A). These mutants either had deletions or scanning mutations. M4A (a.a. 104-199) was kept as a control due to lack of a restriction site at the C-terminus. In M4B (a.a. 119-206), α-helix C was deleted, in M4D (a.a. 104-187) α-helix F was deleted and in M4F (a.a. 119-187) both C and F α-helices were deleted. In M4C (a.a. 104-206), TLLEFYLK residues in α-helix C were mutated to residues AGDATAGA, while in M4E KALGEVD residues in α-helix F were mutated to residues GAHGAVA. Double mutant M4G had the same mutations at C and F α-helices. A similar approach was employed to make mutations in the helices C and F of full length MDA-7/IL-24. In mutant MDA7(C), TLLEFYLK residues in α-helix C were mutated to residues TLAGSRLG. The construct was generated by adding a restriction site XbaI in the middle, by PCR both the 5′ and 3′ DNA fragments were amplified and both the fragments were ligated into HindIII and BamHI sites of pREP4 (see Table 1 for details). In mutant MDA7 (C/F), the same mutations were made in α-helix C and KALGEVD residues in α-helix F were mutated to residues GAHGAVA. For making the mutant MDA7 (C/F), mutant MDA7(C) was used as a template. However MDA7 (C/F) contained 1-199 residues of full-length wild type MDA7/IL-24 due to absence of a restriction endonuclease site at the end. The construct M4A served as a control for MDA7 (C/F) as removal of the last 7 residues had no effect on the activity of M4 (FIG. 3E). All the mutants were cloned into the HindIII and BamHI sites of pREP4. Both mutant MDA7(C) and MDA7 (C/F) were also cloned in vector pCMV3×flag vector to generate flag tagged versions of these proteins in HindIII and BamHI sites of the vector. The authenticity of all the constructs was confirmed by sequence analysis.

Construction of Recombinant Adenoviruses

The construction of Ad.mda-7 (replication incompetent adenovirus expressing mda-7/IL-24) was described previously. Similar approaches were used to construct and purify Ad.M4 (replication incompetent adenovirus expressing the mutant M4) (Holmes et al., 2003; Leszczyniecka et al., 2002). M4 was cloned in the shuttle vector (p0tg-CMV) and the replication defective adenovirus was prepared by homologous recombination with the to E1 and E3 regions deleted parental adenoviral vector in E. coli as described previously (Holmes et al., 2003). Stock virus preparations were diluted in DMEM containing 1% fetal bovine serum and inoculated onto cell monolayers at the indicated plaque forming units (pfu). After 2 h of virus adsorption at 37° C. with regular rotation of plates, the virus inoculum was removed and DMEM containing 10% fetal bovine serum was added to the infected monolayers and cells were incubated at 37° C. for the indicated times. The empty adenoviral vector (Ad.vec) was used as a control.

MTT Assays

Cells were seeded in 96-well tissue culture plates (1.5×10³ cells per well) and next day infected with Ad.vec, Ad.mda-7 and Ad.M4 at multiple pfu's for different time points as described in results. After incubation for specific times, medium was removed and 100 μl of fresh medium containing 0.5 mg/ml 3-(4,5-dimethylthiazol-2-yl)-2,5-diphenyl tetrazolium bromide (MTT) solution was added to each well and incubated for 4 h in a 5% CO2 incubator at 37° C. The precipitate was solubilized in an equal volume of solubilization solution (0.01 N HCl in 10% SDS). After complete mixing plates were read using BioRad multiplate reader Model 550 at 595 nm (Lebedeva et al., 2000). Cell viability was assessed as a ratio between optical densities of treated cells to Ad.vec infected cells. A statistical analysis of the results was performed using the Analysis ToolPack provided by Microsoft Excel. A Student two-sample t test, assuming unequal variances, was used to determine the equality of the means of two samples. The confidence level α was 0.05.

Colony Formation Assays

Cancer cells, HeLa, DU-145 and T47D, and normal cells, P69, FM516-SV and PHFA, were transfected with 20 μg of DNA using either Lipofectamine 2000 (Invitrogen) or Superfect (Qiagen). The next day, cells were subcultured at 1×10⁵ cells for HeLa, Du-145 and T47D, or 2×10⁵ cells for P69, FM515-SV or PHFA in 60-mm dishes and selected for colony forming ability in the presence of various concentrations of hygromycin (400 μg/ml for HeLa, 300 μg/ml for Du-145 and 200 μg/ml for the other cell lines). The amount of hygromycin was standardized prior to performing experiments with the different cell types. Media containing hygromycin was replaced every 4 days. After 2 weeks of incubation, colonies were fixed in 4% formaldehyde and stained with Giemsa. Colonies ≧50 cells were enumerated under a dissection microscope. To determine the effect of adenovirus transduction, cells were infected with 10, 50 or 100 pfu/cell with Ad.vec, Ad.mda-7, Ad.M4 and Ad.mda-7-SP- (adenovirus containing a mutant of mda-7/IL-24 lacking the secretory peptide region). The next day 200-500 cells were seeded to determine colony forming ability. After 2 weeks colonies were fixed, stained and colonies ≧50 cells were enumerated.

RNA Isolation and Northern Blot Analysis

Total RNA was extracted from the cells by using Qiagen RNeasy kit according to the manufacturers' protocol and Northern blotting was performed as described previously (Lebedeva et al., 2002; Leszczyniecka et al., 2002; Su et al., 1998). Fifteen μg of RNA were denatured and electrophoresed in 1.2% agarose gel with 3% formaldehyde, transferred to nylon membranes and sequentially hybridized with ³²P-labeled cDNA probes as described earlier (Su et al., 1998). The cDNA probes were full-length mda-7/IL-24 and human gadd34, gadd153 and gapdh.

Western Blot Analysis

Cells were harvested in radioimmunoprecipitation assay (RIPA) buffer (1×phosphate buffered saline [PBS], 1% NP-40, 0.5% sodium deoxycholate. 0.1% sodium dodecyl sulfate [SDS]) containing a protease inhibitor cocktail (Sigma, St. Louis, Mo.). Protein was quantified using BioRad protein Assay mix and 25-100 μg protein per lane was analyzed on a 12% sodium dodecyl sulfate-polyacrylamide gel electrophoresis (SDS-PAGE). Separated samples were transferred onto a nitrocellulose membrane and the membranes were blocked with 5% milk for 1 h. Membranes were incubated overnight at 4° C. in Tris buffered saline—tween (TBS-T) containing different dilutions of primary antibodies: anti-MDA-7/IL-24 (1:5000 in 5% BSA); anti-phospho p38 (1:1000 in 5% BSA). The membrane was then washed stringently in TBS-T three times followed by incubation for 1 h in 1:5000 dilution of horseradish peroxidase-labeled anti-mouse or anti-rabbit secondary antibody. After washing three times in TBS-T, bands were visualized using ECL Western Blotting kit (Amersham Biosciences, Piscataway, N.J.).

Annexin V Binding assays

Cells were seeded in 6-well plates (5×10⁵ cells per well) and the next day cells to were infected with 100 pfu/cell of the indicated virus. After 24 h cells were trypsinized, washed with complete medium and PBS, resuspended in 500 μl of binding buffer containing 2.5 mM CaCl₂ and stained with FITC labeled Annexin-V (BD Biosciences, Palao-Alto, Calif.) and PI for 15 min at room temperature. Flow cytometry was performed immediately after staining (Lebedeva et al., 2003).

Co-Immunoprecipitation of BiP/Grp78 with MDA-7/IL-24 and Its Mutants

Cells were infected with Ad.vec, Ad.M4 or Ad.mda-7 or transfected with various plasmid (Flag tagged MDA7 or M4 and Myc tagged BiP) constructs in a 100-mm dish and after 48 h cells were rinsed with ice-cold phosphate buffered saline (PBS). Cells were lysed in 1 ml of immunoprecipitation buffer containing 25 mM Tris-Cl pH8.0, 137 mM NaCl, 2.5 mM KCl, 1% Triton X-100 and protease inhibitor mix. Cells were scrapped from the plates, transferred into microfuge tubes and rotated at 4° C. for 30 minutes. Tubes were centrifuged for 10 min at 13,000 rpm and 10 μl of supernatant were incubated with 50% Protein A agarose and rotated at 4° C. for 1 h to eliminate non-specific interactions. Samples were centrifuged and mixed with anti BiP/Grp78 or 9E10 myc monoclonal antibodies (1:200) dilution and rotated overnight at 4° C. Immune-complexes were precipitated with 25 μl of 50% protein A agarose for 2 h. The immonoprecipitates were washed very gently with the IP buffer three times so that the co-immuno precipitating molecules were not lost and at the same time non-specific interactions were inhibited. The immunoprecipitates were resuspended in 50 μl of 10 mM Tris-Cl pH 8.0 and 1 mM EDTA and resolved by SDS-PAGE. The proteins were then transferred to nitrocellulose membranes and probed with primary (anti-MDA-7/IL-24 or anti-M2) antibodies over night followed by probing with horseradish peroxidase-conjugated antibodies specific for the heavy chain of IgG. Secondary antibodies specific for heavy chain were used as size of MDA-7/IL-24 corresponds to the size of light chain of IgG and it interfered with the detection of MDA-7/IL-24. Membranes were also probed with either anti-BiP/GRP78 or 9E10 antibodies to determine the amounts of immunoprecipitates. The dilution of primary antibodies used for immunoblotting was 1:1000 for MDA-7/IL-24, 9E10, BiP/GRP78 as well as anti-FLAG M2 and the dilution of secondary antibodies was 1:10,000. Blots were visualized with ECL reagents (Amersham Biosciences, Piscataway, N.J.).

Immunofluorescence

DU-145 and P69 cells (1×10⁵) were grown on two chamber slides (BD Falcon Biosciences, Bedford, Mass.). The next day, cells were infected with 50 pfu/cell of Ad.mda-7 or Ad.M4 and after 24 h, cells were fixed in 4% paraformaldehyde in PBS for 30 min, permeabilized by 0.1% Triton X-100 in PBS for 10 min. Cells were rinsed in PBS and blocked by 5% BSA in PBS for 2 h and then incubated with anti-MDA-7/IL-24 antibody and the anti-ER protein Calregulin overnight (1:500 dilution of both the antibodies). Cells were washed three times 5 min each in PBS and incubated with FITC conjugated anti-rabbit (for green channel detection) and anti-mouse rhodamine (for red channel detection) secondary antibodies (Molecular Probes) for 2 h at room temperature. Cells were washed three times 5 min each in PBS at room temperature. Slides were mounted and cells were visualized using a Zeiss LSM 510 fluorescence and a 100× objective. For localization of MDA-7/IL-24 deletion mutant protein HeLa, DU-145 or P69 cells were transfected with Flag-tagged DNA constructs and after 24 h of transfection immunostaining was performed as described earlier. Cells were incubated with mouse anti-Flag M2 (Sigma, St. Louis, Mo.) and rabbit anti BiP/GRP78 primary antibodies overnight. FITC conjugated anti mouse and rhodamine conjugated anti rabbit secondary antibodies were used to detect flag tagged MDA-7/IL-24 deletion proteins and BiP/GRP78, respectively.

Animal Tumorigenicity Studies

T47D human breast carcinoma cells (2×10⁶) were injected subcutaneously in 100 μl of PBS in the left and right flanks of male athymic nude mice (NCR^{nu/nu}; 4 weeks old; ˜20 g body weight). After the establishment of visible tumors of ˜75 mm³, requiring ˜4-5 days, intratumoral injections of different Ad were given only to the tumors on the left side at a dose of 1×10⁸ pfu/cell in 100 μl. The injections were given 3 times a week for the first week and then twice a week for two more weeks to a total of seven injections. At least 5 animals were used per experimental point. Tumor volume was measured twice weekly with a caliper and calculated using the formula π6×larger diameter×(smaller diameter)². At the end of the experiment the animals were sacrificed and the tumors were removed and weighed.

### Example 3

### Bystander Antitumor Actvity of mda-7/IL-24 and M4

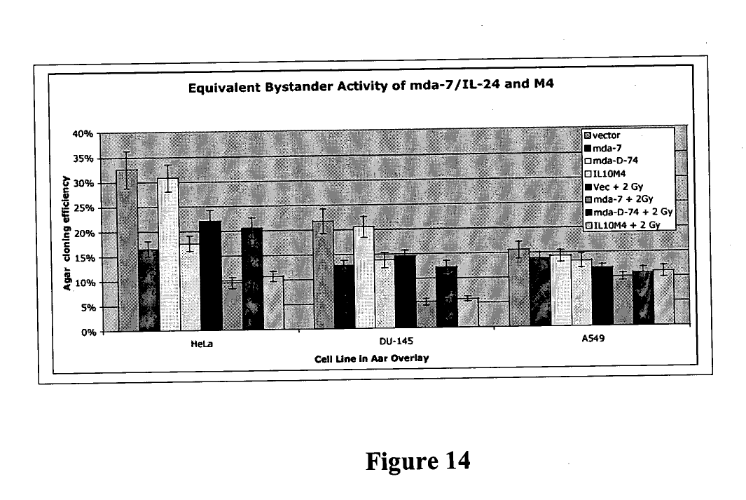

Experimental Design: Previous studies by Su et al. (Oncogene, 2005, 24:7552-7566) indicate that infection of normal cells with Ad.mda-7 results in secretion of MDA-7/IL-24 protein that affects the growth and response to radiation of tumor cells, i.e., a “bystander antitumor” activity. In the experiment shown, early passage primary human fetal astrocytes (PHFA) were seeded in complete growth medium (Dulbecco's modified Eagle's medium supplemented with 10% fetal bovine serum) at 2×10⁵ cells per 60-mm tissue culture plate. The next day the cells were transfected with the indicated expression constructs, vector, mda-7 (an expression construct expressing mda-7/IL-24), mda-D-74 (an expression vector expressing a control gene that is not secreted) or IL10M4 (an expression construct in which the IL-10 secretory signal sequence has been linked to the M4 gene construct), by lipofectamin following the manufacturer's (Invitrogen) instructions. After 24 hr incubation, the transfected cells were overlaid with 5×10⁴ (HeLa) or 1×10⁵ (DU-145 or A549) in 0.4% agar/medium. Forty-eight hr later, the cultures were either irradiated or mock-irradiated with 2 Gy of γ-ray. After 10 days incubation, with agar overlay every 2 days, colonies ≧2 mm in size were counted. The data presented in FIG. 14 is the average of 3 independent plates with S.D. indicated.

Results: Both secreted mda-7/WL-24 and M4 suppress agar growth to a similar extent in HeLa and DU-145 cells (FIG. 14). In contrast, neither mda-7/IL-24 nor M4 suppress growth of A549 cells in agar (which lack canonical IL-20/IL-22 receptors for MDA-7/IL-24 and do not respond to secreted MDA-7/IL-24 with a bystander effect). Additionally, when treated with 2 Gy of radiation, a potentiation of cancer-growth suppression, in HeLa and DU-145 cells, but not in A549 cells, was evident under circumstances where MDA-7/IL-24 or M4 was produced and secreted. Additionally, colony size was generally smaller in cultures containing PHFA cells transfected with mda-7/IL-24 of IL10M4. Both mda-7/IL-24 and M4 display equivalent cancer growth suppressive properties, which is dependent on the presence of canonical IL-20/IL-22 receptors for-MDA-7/IL-24. In addition, as previously documented for MDA-7/IL-24, secretion of M4 also results in enhanced cancer cell growth suppression following radiation treatment. These results indicate that secreted M4 displays similar “bystander antitumor” activity as does secreted MDA-7/IL-24.

Various publications, patents and patent applications are cited herein, the contents of which are hereby incorporated by reference in their entireties.

While the foregoing invention has been described in some detail for purposes of clarity and understanding, these particular embodiments and examples are to be considered as illustrative and not restrictive. It will be appreciated by one skilled in the art from a reading of this disclosure that various changes in form and detail can be made without departing from the true scope of the invention.

### REFERENCES

Bae, V. L., Jackson-Cook, C. K., Brothman, A. R., Maygarden, S. J., and Ware, J. L. (1994). Tumorigenicity of SV40 T antigen immortalized human prostate epithelial cells: association with decreased epidermal growth factor receptor (EGFR) expression. Int J Cancer 58, 721-729.
Blumberg, H., Conklin, D., Xu, W. F., Grossmann, A., Brender, T., Carollo, S., Eagan, M., Foster, D., Haldeman, B. A., Hammond, A., et al. (2001). Interleukin 20: discovery, receptor identification, and role in epidermal function. Cell 104, 9-19.
Chada, S., Bocangel, D., Ramesh, R., Grimm, E. A., Mumm, J. B., Mhashilkar, A. M., and Zheng, M. (2005). mda-7/IL24 kills pancreatic cancer cells by inhibition of the Wnt/PI3K signaling pathways: identification of IL-20 receptor-mediated bystander activity against pancreatic cancer. Mol Ther 11, 724-733.
Cunningham, C. C., Chada, S., Merritt, J. A., Tong, A., Senzer, N., Zhang, Y., Mhashilkar, A., Parker, K., Vukelja, S., Richards, D., et al. (2005). Clinical and local biological effects of an intratumoral injection of mda-7 (IL24; INGN 241) in patients with advanced carcinoma: a phase I study. Mol Ther 11, 149-159.
Dumoutier, L., Leemans, C., Lejeune, D., Kotenko, S. V., and Renauld, J. C. (2001). Cutting edge: STAT activation by IL-19, IL-20 and mda-7 through IL-20 receptor complexes of two types. J Immunol 167, 3545-3549.
Dumoutier, L., and Renauld, J. C. (2002). Viral and cellular interleukin-10 (IL-10)-related cytokines: from structures to functions. Eur Cytokine Netw 13, 5-15.
Fisher, P. B. (2005). Is Mda-7/IL-24 a “Magic Bullet” for Cancer? Cancer Research 65, 10128.
Fisher, P. B., Gopalkrishnan, R. V., Chada, S., Ramesh, R., Grimm, E. A., Rosengeld, M. R., Curiel, D. T., and Dent, P. (2003). mda-7/IL-24, a novel cancer selective apoptosis inducing cytokine gene: from the laboratory into the clinic. Cancer Biol Ther 2, S23-37.
Fry, M. J. (2001). Phosphoinositide 3-kinase signalling in breast cancer: how big a role might it play? Breast Cancer Res 3, 304-312.
Gupta, P., Su, Z.-z., Lebedeva, I., Sarkar, D., Sauane, M., Emdad, L., Bachelor, M., Grant, S., Curiel, D. T., Dent, P., and Fisher, P. B. (2005). mda7/WL-24: Multifuctional Cancer Specific Apoptosis -inducing Cytokine. Pharmacol Ther, In Press. Holmes, M., Rosenberg, E., and Valerie, K. (2003). Adenovirus expressing p53. Methods Mol Biol 234, 1-16.
Huang, E. Y., Madireddi, M. T., Gopalkrishnan, R. V., Leszczyniecka, M., Su, Z., Lebedeva, I. V., Kang, D., Jiang, H., Lin, J. J., Alexandre, D., et al. (2001). Genomic structure, chromosomal localization and expression profile of a novel melanoma differentiation associated (mda-7) gene with cancer specific growth suppressing and apoptosis inducing properties. Oncogene 20, 7051-7063.
Kotenko, S. V. (2002). The family of IL-10-related cytokines and their receptors: related, but to what extent? Cytokine Growth Factor Rev 13, 223-240.
Lebedeva, I., Rando, R., Ojwang, J., Cossum, P., and Stein, C. A. (2000). Bcl-xL in prostate cancer cells: effects of overexpression and down-regulation on chemosensitivity. Cancer Res 60, 6052-6060.
Lebedeva, I. V., Sauane, M., Gopalkrishnan, R. V., Sarkar, D., Su, Z. Z., Gupta, P., Nemunaitis, J., Cunningham, C., Yacoub, A., Dent, P., and Fisher, P. B. (2005a). mda-7/IL-24: exploiting cancer's achilles' heel. Mol Ther 11, 4-18.
Lebedeva, I. V., Su, Z. Z., Chang, Y., Kitada, S., Reed, J. C., and Fisher, P. B. (2002). The cancer growth suppressing gene mda-7 induces apoptosis selectively in human melanoma cells. Oncogene 21, 708-718.
Lebedeva, I. V., Su, Z. Z., Sarkar, D., Gopalkrishnan, R. V., Waxman, S., Yacoub, A., Dent, P., and Fisher, P. B. (2005b). Induction of reactive oxygen species renders mutant and wild-type K-ras pancreatic carcinoma cells susceptible to Ad.mda-7-induced apoptosis. Oncogene 24, 585-596.
Lebedeva, I. V., Su, Z. Z., Sarkar, D., Kitada, S., Dent, P., Waxman, S., Reed, J. C., and Fisher, P. B. (2003). Melanoma differentiation associated gene-7, mda-7/interleukin-24, induces apoptosis in prostate cancer cells by promoting mitochondrial dysfunction and inducing reactive oxygen species. Cancer Res 63, 8138-8144.
Leszczyniecka, M., Kang, D. C., Sarkar, D., Su, Z. Z., Holmes, M., Valerie, K., and Fisher, P. B. (2002). Identification and cloning of human polynucleotide phosphorylase, hPNPase old-35, in the context of terminal differentiation and cellular senescence. Proc Natl Acad Sci USA 99, 16636-16641.
Madireddi, M. T., Dent, P., and Fisher, P. B. (2000). Regulation of mda-7 gene expression during human melanoma differentiation. Oncogene 19, 1362-1368.
Mhashilkar, A. M., Stewart, A. L., Sieger, K., Yang, H. Y., Khimani, A. H., Ito, I., Saito, Y., Hunt, K. K., Grimm, E. A., Roth, J. A., et al. (2003). MDA-7 negatively regulates the beta-catenin and P13K signaling pathways in breast and lung tumor cells. Mol Ther 8, 207-219.
Pestka, S., Krause, C. D., Sarkar, D., Walter, M. R., Shi, Y., and Fisher, P. B. (2004). Interleukin-10 and Related Cytokines and Receptors. Annu Rev Immunol 22, 929-979.
Rao, R. V., Castro-Obregon, S., Frankowski, H., Schuler, M., Stoka, V., del Rio, G., Bredesen, D. E., and Ellerby, H. M. (2002a). Coupling endoplasmic reticulum stress to the cell death program. An Apaf-1-independent intrinsic pathway. J Biol Chem 277, 21836-21842.
Rao, R. V., Ellerby, H. M., and Bredesen, D. E. (2004). Coupling endoplasmic reticulum stress to the cell death program. Cell Death Differ 11, 372-380.
Rao, R. V., Peel, A., Logvinova, A., del Rio, G., Hermel, E., Yokota, T., Goldsmith, P. C., Ellerby, L. M., Ellerby, H. M., and Bredesen, D. E. (2002b). Coupling endoplasmic reticulum stress to the cell death program: role of the ER chaperone GRP78. FEBS Lett 514, 122-128.
Sarkar, D., Su, Z. Z., Lebedeva, I. V., Sauane, M., Gopalkrishnan, R. V., Dent, P., and Fisher, P. B. (2002a). mda-7 (IL-24): signaling and functional roles. Biotechniques Suppl, 30-39.
Sarkar, D., Su, Z. Z., Lebedeva, I. V., Sauane, M., Gopalkrishnan, R. V., Valerie, K., Dent, P., and Fisher, P. B. (2002b). mda-7 (IL-24) Mediates selective apoptosis in human melanoma cells by inducing the coordinated overexpression of the GADD family of genes by means of p38 MAPK. Proc Natl Acad Sci USA 99, 10054-10059.
Sarkar, D., Su, Z. Z., Vozhilla, N., Park, E. S., Gupta, P., and Fisher, P. B. (2005). Dual cancer-specific targeting strategy cures primary and distant breast carcinomas in nude mice. Proc Natl Acad Sci USA 102, 14034-14039.
Sauane, M., Gopalkrishnan, R. V., Choo, H. T., Gupta, P., Lebedeva, I. V., Yacoub, A., Dent, P., and Fisher, P. B. (2004a). Mechanistic aspects of mda-7/1L-24 cancer cell selectivity analysed via a bacterial fusion protein. Oncogene 23, 7679-7690.
Sauane, M., Gopalkrishnan, R. V., Lebedeva, I., Mei, M. X., Sarkar, D., Su, Z. Z., Kang, D. C., Dent, P., Pestka, S., and Fisher, P. B. (2003a). Mda-7/1L-24 induces apoptosis of diverse cancer cell lines through JAK/STAT-independent pathways. J Cell Physiol 196, 334-345.
Sauane, M., Gopalkrishnan, R. V., Sarkar, D., Su, Z. Z., Lebedeva, I. V., Dent, P., Pestka, S., and Fisher, P. B. (2003b). MDA-7/IL-24: novel cancer growth suppressing and apoptosis inducing cytokine. Cytokine Growth Factor Rev 14, 35-51.
Sauane, M., Lebedeva, I. V., Su, Z. Z., Choo, H. T., Randolph, A., Valerie, K., Dent, P., Gopalkrishnan, R. V., and Fisher, P. B. (2004b). Melanoma differentiation associated gene-7/interleukin-24 promotes tumor cell-specific apoptosis through both secretory and nonsecretory pathways. Cancer Res 64, 2988-2993.
Shen, J., Chen, X., Hendershot, L., and Prywes, R. (2002). ER stress regulation of ATF6 localization by dissociation of BiP/GRP78 binding and unmasking of Golgi localization signals. Dev Cell 3, 99-111.
Shen, J., and Prywes, R. (2004). Dependence of site-2 protease cleavage of ATF6 on prior site-I protease digestion is determined by the size of the luminal domain of ATF6. J Biol Chem 279, 43046-43051.
Shen, J., Snapp, E. L., Lippincott-Schwartz, J., and Prywes, R. (2005). Stable binding of ATF6 to BiP in the endoplasmic reticulum stress response. Mol Cell Biol 25, 921-932.
Su, Z., Emdad, L., Sauane, M., Lebedeva, I. V., Sarkar, D., Gupta, P., James, C. D., Randolph, A., Valerie, K., Walter, M. R., et al. (2005a). Unique aspects of mda-7/IL-24 antitumor bystander activity: establishing a role for secretion of MDA-7/IL-24 protein by normal cells. Oncogene.
Su, Z., Lebedeva, I. V., Gopalkrishnan, R. V., Goldstein, N. I., Stein, C. A., Reed, J. C., Dent, P., and Fisher, P. B. (2001). A combinatorial approach for selectively inducing programmed cell death in human pancreatic cancer cells. Proc Natl Acad Sci USA 98, 10332-10337.
Su, Z. Z., Emdad, L., Sarkar, D., Randolph, A., Valerie, K., Yacoub, A., Dent, P., and Fisher, P. B. (2005b). Potential molecular mechanism for rodent tumorigenesis: mutational generation of Progression Elevated Gene-3 (PEG-3). Oncogene. Su, Z. Z., Lebedeva, I. V., Sarkar, D., Gopalkrishnan, R. V., Sauane, M., Sigmon, C., Yacoub, A., Valerie, K., Dent, P., and Fisher, P. B. (2003). Melanoma differentiation associated gene-7, mda-7/IL-24, selectively induces growth suppression, apoptosis and radiosensitization in malignant gliomas in a p53-independent manner. Oncogene 22, 1164-1180.
Su, Z. Z., Madireddi, M. T., Lin, J. J., Young, C. S., Kitada, S., Reed, J. C., Goldstein, N. I., and Fisher, P. B. (1998). The cancer growth suppressor gene mda-7 selectively induces apoptosis in human breast cancer cells and inhibits tumor growth in nude mice. Proc Natl Acad Sci USA 95, 14400-14405.
Tong, A. W., Nemunaitis, J., Su, D., Zhang, Y., Cunningham, C., Senzer, N., Netto, G., Rich, D., Mhashilkar, A., Parker, K., et al. (2005). Intratumoral injection of INGN 241, a nonreplicating adenovector expressing the melanoma-differentiation associated gene-7 (mda-7/IL24): biologic outcome in advanced cancer patients. Mol Ther 11, 160-172.
Vandenbroeck, K., Alloza, I., Brehmer, D., Billiau, A., Proost, P., McFerran, N., Rudiger, S., and Walker, B. (2002). The conserved helix C region in the superfamily of interferon-gamma/interleukin-10-related cytokines corresponds to a high-affinity binding site for the HSP70 chaperone DnaK. J Biol Chem 277, 25668-25676.
Walter, M. R. (2004). Structural analysis of IL-10 and Type I interferon family members and their complexes with receptor. Adv Protein Chem 68, 171-223.
Walter, M. R., and Nagabhushan, T. L. (1995). Crystal structure of interleukin 10 reveals an interferon gamma-like fold. Biochemistry 34, 12118-12125.
Wang, M., Tan, Z., Zhang, R., Kotenko, S. V., and Liang, P. (2002). Interleukin 24 (MDA-7/MOB-5) signals through two heterodimeric receptors, IL-22R1I/L-20R2 and WL-20R1/IL-20R2. J Biol Chem 277, 7341-7347.
Xu, T., Logsdon, N. J., and Walter, M. R. (2004). Crystallization and X-ray diffraction analysis of insect-cell-derived IL-22. Acta Crystallogr D Biol Crystallogr 60, 1295-1298.
Yoshida, H., Haze, K., Yanagi, H., Yura, T., and Mori, K. (1998). Identification of the cis-acting endoplasmic reticulum stress response element responsible for transcriptional induction of mammalian glucose-regulated proteins. Involvement of basic leucine zipper transcription factors. J Biol Chem 273, 33741-33749.

1. An isolated MV1 polypeptide that is from about 145 amino acids to about 175 amino acids in length, and wherein the MV1 polypeptide is at least about 90 percent identical to a region from about amino acid 104 to about amino acid 206 of SEQ ID NO: 2.

2. An isolated MV2 polypeptide that is from about 130 amino acids to about 155 amino acids in length, and wherein the MV2 polypeptide is at least about 90 percent identical to a region from about amino acid 63 to about amino acid 206 of SEQ ID NO: 2.

3. An isolated MV3 polypeptide that is from about 115 amino acids to about 138 amino acids in length, and wherein the MV3 polypeptide is at least about 90 percent identical to a region from about amino acid 80 to about amino acid 206 of SEQ ID NO: 2.

4. An isolated MV4 polypeptide that is from about 90 amino acids to about 110 amino acids in length, and wherein the MV4 polypeptide is at least about 90 percent identical to a region from about amino acid 104 to about amino acid 206 of SEQ ID NO: 2.

5. An isolated MV5 polypeptide that is from about 70 amino acids to about 80 amino acids in length, and wherein the MV5 polypeptide is at least about 90 percent identical to a region from about amino acid 131 to about amino acid 206 of SEQ ID NO: 2.

6. An isolated MV6 polypeptide that is from about 45 amino acids to about 55 amino acids in length, and wherein the MV6 polypeptide is at least about 90 percent identical to a region from about amino acid 159 to about amino acid 206 of SEQ ID NO: 2.

7. An isolated MV7 polypeptide that is from about 122 amino acids to about 146 amino acids in length, and wherein the MV7 polypeptide is at least about 90 percent identical to a region from about amino acid 48 to about amino acid 180 of SEQ ID NO: 2.

8. An isolated MV8 polypeptide that is from about 100 amino acids to about 120 amino acids in length, and wherein the MV8 polypeptide is at least about 90 percent identical to a region from about amino acid 48 to about amino acid 158 of SEQ ID NO: 2.

9. An isolated MV9 polypeptide that is from about 75 amino acids to about 90 amino acids in length, and wherein the MV9 polypeptide is at least about 90 percent identical to a region from about amino acid 48 to about amino acid 130 of SEQ ID NO: 2.

10. An isolated MV10 polypeptide that is from about 53 amino acids to about 63 amino acids in length, and wherein the MV10 polypeptide is at least about 90 percent identical to a region from about amino acid 48 to about amino acid 104 of SEQ ID NO: 2.

11. An MVAB polypeptide that is from about 32 amino acids to about 59 amino acids in length, and wherein the MVAB polypeptide is at least about 90 percent identical to a region from about amino acid 63 to about amino acid 101 of SEQ ID NO: 2.

12. An MVEF polypeptide that is from about 35 amino acids to about 60 amino acids in length, and wherein the MVEF polypeptide is at least about 90 percent identical to a region from about amino acid 159 to about amino acid 201 of SEQ ID NO: 2.

13. The peptide of claim 1 linked to a stabilizing molecule.

14. The peptide of claim 13, where the stabilizing molecule is a protein.

15. The peptide of claim 14, where the stabilizing molecule is a Glutathione-S-Transferase (GST) protein.

16. A nucleic acid encoding a polypeptide of claim 1.

17. A method of modulating proliferation of a cell, comprising administering, to the cell, an effective amount of a peptide of claim 1.

18. A method for modulating proliferation of a cell, comprising introducing into the cell, a nucleic acid of claim 16.

19. A method for inhibiting proliferation of a cell, the method comprising introducing into the cell an effective amount of the peptide of claim 3.

20. A method for inhibiting cell growth in a subject suffering from a cell proliferative disorder, the method comprising administering an effective, amount of the polypeptide of claim 3 to the subject.

21. The method of claim 20, wherein the disorder is cancer.

22. The method of claim 20, wherein the cell is a tumor cell.

23. A method for inhibiting proliferation of a cell, the method comprising introducing into the cell an effective amount of a nucleic acid encoding the peptide of claim 3.

24. A method for inhibiting cell growth in a subject suffering from a cell proliferative disorder, the method comprising administering an effective, amount of a nucleic acid encoding the polypeptide of claim 3 to the subject.

25. The method of claim 24, wherein the administration of the nucleic acid is via a nucleic acid vector, or a liposome.

26. The method of claim 24, wherein the administration of the nucleic acid is via a virus, a replication defective viral vector, a replication conditional viral vector, a non-integrating virus, an adenovirus, AAV, VSV, Epstein Barr virus, measles, an integrating virus, a lentiviruses, a retroviruses, a plasmid, a synthetic delivery system, a liposome, a cationic polymer, a dendritic cell, a stem cell, or any combination thereof.

27. The method of claim 24, wherein the method further comprises administering to the subject: a chemotherapeutic agent, a generator of free radicals, radiation therapy, an anti-ras agent, an anti-cancer antibody, or an anti-proliferative agent in combination with the polypeptide.

28. A method for treating inflammation in a subject, the method comprising administering, to the subject, an effective amount of the polypeptide of claim 2.

29. A method for treating inflammation in a subject, the method comprising administering, to the subject, an effective amount of a nucleic acid encoding the polypeptide of claim 2.

30. The method of claim 28, wherein the method further comprises administering to the subject an anti-inflammatory agent in combination with the polypeptide.

31. An antibody that specifically binds to the polypeptide of claim 1.

32. The MV1 polypeptide of claim 1, having an amino acid sequence of SEQ ID NO: 3.

33. The MV2 polypeptide of claim 2, having an amino acid sequence of SEQ ID NO: 4.

34. The MV3 polypeptide of claim 3, having an amino acid sequence of SEQ ID NO: 5.

35. The MV4 polypeptide of claim 4, having an amino acid sequence of SEQ ID NO: 6.

36. The MV5 polypeptide of claim 5, having an amino acid sequence of SEQ ID NO: 7.

37. The MV6 polypeptide of claim 6, having an amino acid sequence of SEQ ID NO: 8.

38. The MV7 polypeptide of claim 7, having an amino acid sequence of SEQ ID NO: 9.

39. The MV8 polypeptide of claim 8, having an amino acid sequence of SEQ ID NO: 10.

40. The MV9 polypeptide of claim 9, having an amino acid sequence of SEQ ID NO: 11.

41. The MV10 polypeptide of claim 10, having an amino acid sequence of SEQ ID NO: 12.

42. The MVAB polypeptide of claim 11, having an amino acid sequence of SEQ ID NO: 13.

43. The MVEF polypeptide of claim 12, having an amino acid sequence of SEQ ID NO: 14.

44. A peptidomimetic of the polypeptide of claim 1.

45. A nucleic acid encoding a polypeptide of claim 1 linked to a nucleic acid encoding a secretory peptide.

46. The nucleic acid of claim 45, wherein the nucleic acid is under the control of a promoter and wherein the nucleic acid is linked to a conditionally replicable vector.

47. The nucleic acid of claim 45, wherein the nucleic acid encodes the MV4 polypeptide and where the secretory peptide comprises a secretory peptide of wild-type MDA-7, a cleavage signal peptide of gamma-interferon, an amino terminal leader sequence of mouse immunoglobulin light chain precursor.

48. The nucleic acid of claim 45, wherein the nucleic acid is linked to a conditionally replicating viral vector.

49. The nucleic acid of claim 45, wherein the nucleic acid is linked to a replication deficient viral vector.

50. The nucleic acid of claim 45, wherein the nucleic acid is contained within a liposome.

51. A composition comprising the polypeptide of claim 1.

52. A composition comprising a nucleic acid encoding a polypeptide of claim 1.

53. A host cell containing a nucleic acid molecule encoding any of the polypeptides of claim 1, wherein the nucleic acid is operably linked to a promoter and is expressed by the cell.

54. The host cell of claim 53, wherein the host cell is a dendritic cell or a stem cell.

55. A host cell containing a nucleic acid molecule encoding a polypeptide of claim 1, linked to a second nucleic acid encoding a secretory peptide, wherein the first and second nucleic acids are operably linked to a promoter and the first and second nucleic acids are expressed and secreted by the cell.

56. The host cell of claim 55, wherein the host cell is a dendritic cell or a stem cell.

57. A method for treating a tumor in a subject, the method comprising introducing into cells of a subject a nucleic acid encoding a polypeptide of claim 3, and a secretory peptide so that the cells express and secrete the polypeptide of claim 3 and wherein the expression and secretion of the polypeptide induces transformed-cell specific apoptosis.

58. The method of claim 57, wherein the secretory peptide comprises a secretory peptide selected from the group consisting of: a secretory peptide of wild-type MDA-7, a cleavage signal peptide of gamma-interferon, and an amino terminal leader sequence of mouse immunoglobulin light chain precursor.

59. A method for inducing an anti-tumor bystander activity from a cell, the method comprising introducing into a cell a nucleic acid encoding a polypeptide of claim 3 and a secretory peptide under the control of a promoter, so that the cell expresses and secretes the polypeptide of claim 3, and wherein the expression and secretion of the polypeptide induces bystander anti-tumor activity.

60. The method of claim 59, wherein the cell into which the nucleic acid is introduced is a normal cell.

61. A method for inducing anti-tumor apoptosis in a subject, the method comprising introducing into tumor cells of a subject a nucleic acid encoding a polypeptide of claim 3, wherein the expression of the polypeptide induces anti-tumor apoptosis in the subject.

62. A method for inhibiting angiogenesis in a tumor, the method comprising introducing into one or more cells of the tumor a nucleic acid encoding a polypeptide of claim 3.

63. A method for enhancing activity of an anti-cancer treatment regime of a subject, the method comprising administering to the subject a polypeptide of claim 3 in combination with the anti-cancer treatment regime.

64. The method of claim 63, wherein the anti-cancer treatment regime comprises radiation, monoclonal antibody therapy, chemotherapy, or radioisotope therapy.

65. An anti-idiotypic antibody that specifically binds to Bip/GRP78 in the same way that M4, a polypeptide having the amino acid sequence shown in SEQ ID NO: 6, binds to Bip/GRP78.

66. A polypeptide comprising M4, a polypeptide having the amino acid sequence shown in SEQ ID NO: 6, linked to an amino acid sequence of glutathione-S-transerfase.

67. A method for identifying a compound capable of acting as a surrogate of M4 (SEQ ID NO: 6) by binding to Bip/GRP78 intracellularly, the method comprising:
(a) contacting a cell with a test compound, wherein the cell expresses Bip/GRP78;
(b) determining whether p38 MAPK is activated, wherein the activation of p38 MAPK indicates that the test compound acts as a surrogate of M4 (SEQ ID NO: 6).

68. The method of claim 67, wherein the determination of whether p38 MAPK is activated comprises a determination of whether p38 MAPK is phosphorylated.

69. A method for inducing anti-tumor apoptosis in a subject, the method comprising introducing into tumor cells of a subject a nucleic acid encoding a polypeptide of SEQ ID NO:6, or the polypeptide of SEQ ID NO: 6 linked to glutathione-S-transferase, wherein the expression of the polypeptide induces anti-tumor apoptosis in the subject.

70. A method for inhibiting angiogenesis in a tumor, the method comprising introducing into one or more cells of the tumor a nucleic acid encoding a polypeptide of SEQ ID NO:6, or the polypeptide of SEQ ID NO: 6 linked to glutathione-S-transferase.

71. A method for enhancing activity of an anti-cancer treatment regime of a subject, the method comprising administering to the subject a polypeptide of SEQ ID NO:6, or the polypeptide of SEQ ID NO: 6 linked to glutathione-S-transferase in combination with the anti-cancer treatment regime.

72. The method of claim 71, wherein the anti-cancer treatment regime comprises radiation, monoclonal antibody therapy, chemotherapy, or radioisotope therapy.

73. A method for inducing an anti-tumor bystander activity from a cell, the method comprising introducing into a cell a nucleic acid encoding (a) a polypeptide of SEQ ID NO:6, or the polypeptide of SEQ ID NO: 6 linked to glutathione-S-transferase, and (b) a secretory polypeptide, both (a) and (b) under the control of a promoter, so that the cell expresses and secretes the polypeptide, and wherein the expression and secretion of the polypeptide induces bystander anti-tumor activity.

74. A method for stimulating the immune system to produce additional cytokines, such as interferon gamma, TNF-alpha and interleukin-6 and downregulates TGF-beta, the method comprising administering to a subject in need thereof an effective amount of a polypeptide of SEQ ID NO:6 (M4), or the polypeptide of SEQ ID NO: 6 linked to glutathione-S-transferase.

75. The method of claim 69, wherein the administration of the nucleic acid comprises administration via a virus, a replication defective viral vector, a replication conditional viral vector, a non-integrating virus, an adenovirus, AAV, VSV, Epstein Barr virus, measles, an integrating virus, a lentiviruses, a retroviruses, a plasmid, a synthetic delivery system, a liposome, a cationic polymer, a dendritic cell, a stem cell, or any combination thereof.
